(19) United States
(12) Patent Application Publication
(10) Pub. No.: US 2007/0178133 A1
(43) Pub. Date: Aug. 2, 2007

(54) Medical device, materials, and methods

(75) Inventor: Jason P. Rolland, Durham, NC (US)

Correspondence Address: MORGAN, LEWIS & BOCKIUS LLP, 1701 MARKET STREET, PHILADELPHIA, PA, 19103-2921, US

(73) Assignee: Liquidia Technologies, Inc.

(21) Appl. No.: 11/595,533

(22) Filed: Nov. 9, 2006

Related U.S. Application Data
(60) Provisional application No. 60/734,880, filed on Nov. 9, 2005.

(51) Int. Cl. A61F 2/02; C08F 20/00

(52) U.S. Cl. 424/423; 525/440

(57) ABSTRACT

Dual thermal and photo curable materials are used for fabricating, functionalizing, and utilizing devices, such as medical devices, surgical devices, and medical implants. The materials include thermal and photo curable components that can adhere layers of the materials to one another, to other substrates, or to biologic tissues to form medical devices, surgical devices, and medical implants.

### CROSS REFERENCE TO RELATED APPLICATIONS

This application is based on and claims priority to U.S. Provisional Application No. 60/734,880, filed Nov. 9, 2005, which is incorporated herein by reference in its entirety.

### GOVERNMENT INTEREST

This invention was made with U.S. Government support from Office of Naval Research No. N000140210185 and STC program of the National Science Foundation under Agreement No. CHE-9876674. The U.S. Government has certain rights in the invention.

### TECHNICAL FIELD

Generally, the present invention relates to functional materials and their use for fabricating and functionalizing medical devices and implants.

### ABBREVIATIONS

AC=alternating current
Ar=Argon
° C.=degrees Celsius
cm=centimeter
8-CNVE=perfluoro(8-cyano-5-methyl-3,6-dioxa-1-octene)
CSM=cure site monomer
CTFE=chlorotrifluoroethylene
g=grams
h=hours
1-HPFP=1,2,3,3,3-pentafluoropropene
2-HPFP=1,1,3,3,3-pentafluoropropene
HFP=hexafluoropropylene
HMDS=hexamethyldisilazane
IL=imprint lithography
IPDI=isophorone diisocyanate
MCP=microcontact printing
Me=methyl
MEA=membrane electrode assembly
MEMS=micro-electro-mechanical system
MeOH=methanol
MIMIC=micro-molding in capillaries
mL=milliliters
mm=millimeters
mmol=millimoles
Mₙ=number-average molar mass
m.p.=melting point
mW=milliwatts
NCM=nano-contact molding
NIL=nanoimprint lithography
nm=nanometers
Pd=palladium
PAVE perfluoro(alkyl vinyl) ether
PDMS=poly(dimethylsiloxane)
PEM=proton exchange membrane
PFPE=perfluoropolyether
PMVE perfluoro(methyl vinyl) ether
PPVE perfluoro(propyl vinyl) ether
PSEPVE=perfluoro-2-(2-fluorosulfonylethoxy)propyl vinyl ether
PTFE=polytetrafluoroethylene
SAMIM=solvent-assisted micro-molding
SEM=scanning electron microscopy
Si=silicon
TFE=tetrafluoroethylene
μm=micrometers
UV=ultraviolet
W=watts

### BACKGROUND

Many devices, such as surgical instruments, medical devices, prosthetic implants, orthopedic implants, contact lenses, and the like, (“medical devices”) are formed from polymeric materials. Polymeric materials commonly used in the medical device industry include polyurethanes, polyolefins (e.g., polyethylene and polypropylene), poly(meth)acrylates, polyesters (e.g., polyethyleneterephthalate), polyamides, polyvinyl resins, silicone resins (e.g., silicone rubbers and polysiloxanes), polycarbonates, polyfluorocarbon resins, synthetic resins, polystyrene, various bioerodible materials, and the like. Although these and other materials commonly used have proven to be useful there are many drawbacks with the materials and the devices fabricated therefrom. Perfluoropolyether (“PFPE”) has recently been disclosed as a further polymer for use in medical devices. PFPE materials provide benefits such as low surface energy, highly inert surfaces, oxygen permeability, bacteria impermeable, and the like, such as disclosed in U.S. patent applications 2005/0142315 A1; 2005/0271794 A1; and 2005/0273146 A1, each of which are incorporated herein by reference in their entirety. However, drawbacks remain with devices fabricated from or partially incorporating polymer materials.

A current drawback of medical devices fabricated from or incorporating a polymer is the lack of ability to fabricate devices from multiple layers or in multiple components and easily and safely adhere the layers/components to each other. Another drawback is that with any implant there is always the chance of bio-fouling on the surface of the implant. Bio-fouling can occur due to the tissue/implant interface gap and/or the surface characteristics of the implant material. Accordingly, a need exists for improving the polymeric materials, functionalizing the materials, or texturing the surface of medical device materials to generate a better tissue/device interface and reduce bio-fouling.

### SUMMARY

The present invention describes a medical device configured to be implanted into a patient, where the device includes a reaction product of a first cure and is capable of a second reaction cure. The present invention also describes a medical device configured to be implanted into a patient, where the device includes a reaction product of a first cure and is capable of a second cure. In some embodiments, the medical device includes a polymer and in some embodiments, the polymer includes a fluorinated polymer. In some embodiments, the polymer is selected from a perfluoropolyether or a poly(dimethylsiloxane).

According to some embodiments, the first cure includes exposing the device to actinic radiation or to thermal energy. In some embodiments, the second cure includes exposing the device to actinic radiation or to thermal energy. In alternative embodiments, the medical device includes a reaction product of a methacrylate, an acrylate, an epoxy, or a free radical polymerization. In alternative embodiments, the medical device includes a thermoplastic material, an organic material, an imaging agent, a drug, a treatment agent, an antibiotic, biologic material, a soluble material, a biodegradable material, a hydrophilic material, a hydrophobic material, an inorganic material, a ceramic, a metal, or a porogen. According to some embodiments, the medical device includes a coating where the coating can include a fluorinated polymer or a perfluoropolyether.

In other embodiments the present invention includes a medical implant composed of a base material in combination with a first curable functional group and a second curable functional group. According to some embodiments, the base material includes a polymer, a fluorinated polymer, a perfluoropolyether, or a poly(dimethylsiloxane).

In some embodiments, the first curable functional group includes a functional group that reacts upon exposure to actinic radiation and in other embodiments the first curable functional group includes a functional group that reacts upon exposure to thermal energy. In some embodiments, the second curable functional group includes a functional group that reacts upon exposure to actinic radiation and in other embodiments the second curable functional group includes a functional group that reacts upon exposure to thermal energy.

According to some embodiments, the first curable functional group includes a first end-cap, where the first end-cap reacts at a first wavelength, and the second curable functional group includes a second end-cap where the second end-cap reacts at a second wavelength. In some embodiments, first curable functional group of the medical device includes a first end-cap where the first end-cap reacts at a first temperature and the second curable functional group includes a second end-cap, where the second end-cap reacts at a second temperature. In alternative embodiments, the first and second curable functional groups include different end-caps, such as photocurable diurethane methacrylate, diisocyanate, diepoxy, diamine, photocurable diepoxy, or tetrol. According to some embodiments, the medical implant further includes a third curable functional group. The combinations of functional groups can include a first curable functional group of a photocurable diurethane methacrylate, a second curable functional group of a diisocyanate, and a third curable functional group of a tetrol. In other embodiments the combinations of functional groups can include a first curable functional group of a photocurable diurethane methacrylate, a second curable functional group of a diepoxy, and a third curable functional group of a diamine. In yet further embodiments, the functional groups of the medical implant can include a first curable functional group of a photocurable diurethane methacrylate and a second curable functional group of a photocurable diepoxy. In further embodiments, the functional groups can include a first curable functional group of a photocurable diurethane methacrylate and a second curable functional group of a diisocyanate.

According to other embodiments, an apparatus can include a medical article including a medical device having a coating on the medical device, where the coating is a base material in combination with a photocurable functional group and a thermal curable functional group. In alternative embodiments, the coating can include a patterned texture on a surface of the coating. In some embodiments, the patterned texture is configured and dimensioned to interface with a biological tissue and the patterned structure can reduce wettability of the surface and reduce bio-fouling of the surface. According to some embodiments, the patterned texture includes structures of between about 1 nm and about 500 nm protruding from or recessed into the surface. In alternative embodiments, the patterned texture includes structures of less than about 1 micron protruding from or recessed into the surface or structures of between about 5 micron and about 10 micron protruding from or recessed into the surface. In some embodiments, the patterned texture includes a repetitive pattern and the pattern can be a repeating diamond shaped pattern. According to some embodiments, the coating includes a fluorinated polymer or a perfluoropolyether.

In some embodiments of the present invention, an artificial joint can be fabricated from the materials and methods described herein and can include a base material having a photocurable functional group and a thermal curable functional group, where the base material is configured to replace or augment a portion of a natural joint. In some embodiments the base material is configured and dimensioned to replace an articular surface of the joint and in other embodiments the base material is configured and dimensioned to replace a structural component of a natural joint.

According to some embodiments, a medical repair device includes a base material having a photocurable functional group and a thermal curable functional group, where the base material is configured as a patch to interface with a biologic tissue.

The present invention also discloses methods of making and using medical devices and includes a method of repairing a joint, by forming a component of a joint from a base material, where the base material includes a first curable functional group and a second curable functional group, and where the component of the joint is formed by treating the base material with a first cure such that the first curable functional group is activated; and treating the component of the joint with a second cure, where the second cure activates the second curable functional group. According to some embodiments, before the joint is treated with a second cure, the component is implanted to an implant site in a patient. In some embodiments, during the second cure, the component binds with biologic tissue near the implant site and in other embodiments, during the second cure, the component binds with a polymeric material associated with the implant site.

In some embodiments, a method of repairing a tissue includes forming a patch from a base material, where the base material includes a first curable functional group and a second curable functional group, and where the patch is formed by treating the base material with a first cure such that the first curable functional group is activated. Next the patch is applied to a tissue having a defect and the patch is treated with a second cure, wherein the second cure activates the second curable functional group. In some embodiments, the patch is treated with a second cure binds the patch with tissue to be treated. In other embodiments, the patch is treated with a second cure binds the patch with a second polymeric material associated with the tissue to be treated.

According to some embodiments of the present invention, a method of making a medical device includes forming a first component of a medical device from a base material, wherein the base material includes a first curable functional group and a second curable functional group, wherein the first component of the medical device is formed by treating a first quantity of the base material with a first cure such that the first curable functional group is activated. Next, a second component of the medical device is formed from a second quantity of the base material by treating the second quantity with a first cure such that the first curable functional group is activated and the second component is positioned with respect to the first component. Finally, the combined first and second components are treated with a second cure, wherein the second cure activates the second curable functional groups of the components and couples the first and second components together. In some embodiments, the medical device is formed in situ. In some embodiments, the medical device is formed in vitro. According to some embodiments, the medical device is selected from the group of an orthopedic device, a vascular device, a surgical device, a wound repair device, an ocular device, an auditory device, a percutaneous device, an external fixation device, a cosmetic augmentation device, an organ scaffold device, a respiratory device, a gastro-intestinal device, a digestive device, an excretion device, a dermatological device, and the like.

According to other embodiments of the present invention, a method of patching a device includes forming a patch from a base material where the base material includes a first curable functional group and a second curable functional group and where the patch is formed by treating the base material with a first cure such that the first curable functional group is activated. Next, the patch is applied to a device having a defect, and treated with a second cure, where the second cure activates the second curable functional group and couples the patch with the device.

### BRIEF DESCRIPTION OF THE DRAWINGS

FIGS. 1A-1C shows a series of schematic end views depicting the formation of a patterned layer of material according to an embodiment of the present invention;

FIGS. 2A-2D are a series of schematic end views depicting the formation of a device comprising two patterned layers of a material according to an embodiment of the present invention;

FIGS. 3A-3C are schematic representations of adhering a functional device to a treated substrate according to an embodiment of the present invention;

FIGS. 4A-4C are schematic representations of a multilayer device according to an embodiment of the present invention;

FIGS. 5A and 5B are schematic representations of functionalizing the interior surface of a channel according to an embodiment of the present invention;

FIG. 5A is a schematic representation of functionalizing the interior surface of a channel according to an embodiment of the present invention;

FIG. 5B is a schematic representation of functionalizing a surface of a device according to an embodiment of the present invention;

FIGS. 6A-6D are schematic representations of fabricating a microstructure using a degradable and/or selectively soluble material according to an embodiment of the present invention;

FIGS. 7A-7C are schematic representations of fabricating complex structures in a device using degradable and/or selectively soluble materials according to an embodiment of the present invention;

FIG. 8 is a schematic plan view of a device according to an embodiment of the present invention;

FIG. 9 is a schematic of an integrated micro fluid system according to an embodiment of the present invention;

FIG. 10 is a schematic view of a system for flowing a solution or conducting a chemical reaction in a micro device according to an embodiment of the present invention;

FIGS. 11a-11e illustrate a process for fabricating a device according to an embodiment of the present invention;

FIGS. 12A-12B are photomicrographs of an air-actuated pneumatic valve in a presently disclosed PFPE micro device actuated at a pressure of about 45 psi, FIG. 12A is a photomicrograph of an open valve and FIG. 12B is a photomicrograph of a valve closed at about 45 psi;

FIG. 13 shows fabrication of a device from materials and methods of an embodiment of the present invention;

FIG. 14 shows a system for patching a disrupted component using materials and methods of an embodiment of the present invention;

FIG. 15 shows molding and reconstruction of a molded object according to an embodiment of the present invention; and

FIGS. 16A-16C shows reproduction of a device with a lumen according to an embodiment of the present invention.

### DETAILED DESCRIPTION

The presently disclosed subject matter provides materials and methods for use in forming a medical or surgical device and for imparting chemical functionality to a medical or surgical device. In some embodiments, the presently disclosed methods include introducing chemical functionalities that promote and/or increase adhesion between layers of a medical or surgical device. In some embodiments, the chemical functionalities promote and/or increase adhesion between a layer of the device and another surface. Accordingly, in some embodiments, the presently disclosed subject matter provides a method for adhering two-dimensional and three-dimensional structures to a substrate. In some embodiments, the present invention discloses bonding a perfluoropolyether (PFPE) material to other materials, such as a poly(dimethyl siloxane) (PDMS) material, a polyurethane material, a silicone-containing polyurethane material, and a PFPE-PDMS block copolymer material. Thus, in some embodiments, the present invention provides a polymer hybrid device, for example, a device including a perfluoropolyether layer adhered to a polydimethylsiloxane layer, a polyurethane layer, a silicone-containing polyurethane layer, and/or a PFPE-PDMS block copolymer layer. U.S. Pat. Nos. 3,810,874; 3,810,875; 4,094,911; and 4,440,918 disclose synthesis of functional PFPE's, each reference is incorporated herein by reference in its entirety.

In some embodiments, a chemical functionality of the device material is adjusted to attach a polymer, biopolymer, small organic “switchable” molecule, inorganic composition, or small molecule that can affect the device material properties such as, for example, hydrophobicity, reactivity, or the like. In some embodiments, the material includes degradable or selectively soluble polymers or pore forming agents such that the materials degrade in a predetermined manner or rate.

Unless otherwise defined, all technical and scientific terms used herein have the same meaning as commonly understood by one of ordinary skill in the art to which this presently described subject matter belongs. All publications, patent applications, patents, and other references mentioned herein are incorporated by reference in their entirety. Throughout the specification and claims, a given chemical formula or name shall encompass all optical and stereoisomers, as well as racemic mixtures where such isomers and mixtures exist.

I. Definitions

As used herein, the term “pattern” can include micro and/or nano recesses and/or projections of or from a surface. The pattern can be regular or irregular, symmetric or asymmetric, or the like.

As used herein, the term “intersect” can mean to meet at a point, to meet at a point and cut through or across, or to meet at a point and overlap. More particularly, as used herein, the term “intersect” describes an embodiment wherein two channels meet at a point, meet at a point and cut through or across one another, or meet at a point and overlap one another. Accordingly, in some embodiments, two channels can intersect, i.e., meet at a point or meet at a point and cut through one another, and be in fluid communication with one another. In some embodiments, two channels can intersect, i.e., meet at a point and overlap one another, and not be in fluid communication with one another, as is the case when a flow channel and a control channel intersect.

As used herein, the term “communicate” (e.g., a first component “communicates with” or “is in communication with” a second component) and grammatical variations thereof are used to indicate a structural, functional, mechanical, electrical, optical, or fluidic relationship, or any combination thereof, between two or more components or elements. As such, the fact that one component is said to communicate with a second component is not intended to exclude the possibility that additional components can be present between, and/or operatively associated or engaged with, the first and second components.

As used herein, the term “monolithic” refers to a structure having or acting as a single, uniform structure.

As used herein, the term “non-biological organic materials” refers to organic materials, i.e., those compounds having covalent carbon-carbon bonds, other than biological materials. As used herein, the term “biological materials” includes nucleic acid polymers (e.g., DNA, RNA) amino acid polymers (e.g., enzymes, proteins, and the like) and small organic compounds (e.g., steroids, hormones) wherein the small organic compounds have biological activity, especially biological activity for humans or commercially significant animals, such as pets and livestock, and where the small organic compounds are used primarily for therapeutic or diagnostic purposes. While biological materials are of interest with respect to pharmaceutical and biotechnological applications, a large number of applications involve chemical processes that are enhanced by other than biological materials, i.e., non-biological organic materials.

As used herein, the term “photocured” refers to the reaction of polymerizable groups whereby the reaction can be triggered by actinic radiation, such as UV light. In this application UV-cured can be a synonym for photocured.

As used herein, the term “thermal cure” or “thermally cured” refers to the reaction of polymerizable groups, whereby the reaction can be triggered by heating the material beyond a threshold.

Following long-standing patent law convention, the terms “a”, “an”, and “the” refer to “one or more” when used in this application, including the claims. Thus, for example, reference to “a microfluidic channel” includes a plurality of such microfluidic channels, and so forth.

II. Materials

In certain embodiments, the presently disclosed subject matter broadly describes and employs solvent resistant, low surface energy polymeric materials. According to some embodiments the low surface energy polymeric materials include, but are not limited to perfluoropolyether (PFPE), poly(dimethylsiloxane) (PDMS), poly(tetramethylene oxide), poly(ethylene oxide), poly(oxetanes), polyisoprene, polybutadiene, fluoroolefin-based fluoroelastomers, and the like. An example of casting a device with such materials includes casting or molding liquid PFPE precursor materials onto a patterned substrate and then curing the liquid PFPE precursor materials to generate a patterned layer of functional PFPE material, which can be used to form a device, such as a medical or surgical device. For simplification purposes, most of the description will focus on PFPE materials, however, it should be appreciated that other such polymers, such as those recited above, can be utilized with the methods, materials, and devices of the present invention.

In some embodiments, the low surface energy polymeric material of the present invention includes solvent resistant properties. In some embodiments, the solvent resistant properties result from the fluorinated based materials of the present invention. As used herein, the term “solvent resistant” refers to materials, such as elastomeric material that neither swells nor dissolves in common hydrocarbon-based organic solvents or acidic or basic aqueous solutions. Representative fluorinated elastomer-based materials include but are not limited to perfluoropolyether (PFPE)-based materials.

In certain embodiments, the PFPE materials exhibit desirable properties for use in medical and/or surgical devices. For example, functional PFPE materials typically have a low surface energy, are non-toxic, UV and visible light transparent, highly gas permeable; cure into a tough, durable, highly fluorinated elastomeric or glassy materials with excellent release properties, resistant to swelling, solvent resistant, biocompatible, combinations thereof, and the like. The properties of these materials can be tuned over a wide range through the judicious choice of additives, fillers, reactive co-monomers, functionalization agents, curing additives, and the like, examples of which are described further herein. Such properties that are desirable to modify, include, but are not limited to, modulus, tear strength, surface energy, permeability, functionality, mode of cure, solubility, toughness, hardness, surface properties and functionality, binding characteristics, elasticity, swelling characteristics, porosity, combinations thereof, and the like.

Some examples of methods of adjusting mechanical and or chemical properties of the finished material includes, but are not limited to, shortening the molecular weight between cross-links to increase the modulus of the material, adding monomers that form polymers of high Tg to increase the modulus of the material, adding charged monomer or species to the material to increase the surface energy or wettability of the material, combinations thereof, and the like. According to one embodiment, the surface energy is below about 30 mN/m. According to another embodiment the surface energy is between about 7 mN/m and about 20 mN/m. According to a more preferred embodiment, the surface energy is between about 10 mN/m and about 15 mN/m. The non-swelling nature and easy release properties of the presently disclosed PFPE materials enhance fabrication capabilities and functionality of medical or implantable articles or devices that contain these materials.

### II.A. Perfluoropolyether Materials Prepared from a Liquid PFPE Precursor Material Having a Viscosity Less than about 100 Centistokes

As would be recognized by one of ordinary skill in the art, perfluoropolyethers (PFPEs) have been in use for over 25 years for selective applications, such as lubricants. Commercial PFPE materials are made by polymerization of perfluorinated monomers. The first member of this class can be made by cesium fluoride catalyzed polymerization of hexafluoropropene oxide (HFPO) yielding a series of branched polymers designated as KRYTOX® (DuPont, Wilmington, Del., United States of America). A similar polymer is produced by the UV catalyzed photo-oxidation of hexafluoropropene (FOMBLIN® Y) (Solvay Solexis, Brussels, Belgium). Further, a linear polymer (FOMBLIN® Z) (Solvay) is prepared by a similar process, but utilizing tetrafluoroethylene. Finally, a fourth polymer (DEMNUM®) (Daikin Industries, Ltd., Osaka, Japan) is produced by polymerization of tetrafluorooxetane followed by direct fluorination.

Structures for the PFPE fluids are presented in Table I. Table II contains property data for some members of the PFPE class of liquids. Likewise, the physical properties of functional PFPEs are provided in Table III. In addition to these commercially available PFPE fluids, a new series of structures are being prepared by direct fluorination technology. Representative structures of these new PFPE materials appear in Table IV. Of the abovementioned PFPE fluids, only KRYTOX® and FOMBLIN® Z have been extensively used in applications. See Jones. W. R. Jr., The Properties of Perfluoropolyethers Used for Space Applications, NASA Technical Memorandum 106275 (July 1993), which is incorporated herein by reference in its entirety. Accordingly, the use of such PFPE materials is provided in the presently disclosed subject matter.

TABLE I
NAMES AND CHEMICAL STRUCTURES
OF COMMERCIAL PFPE FLUIDS

| NAME | Structure |
|---|---|
| DEMNUM ® | C₃F₇O(CF₂CF₂CF₂O)ₓC₂F₅ |
| KRYTOX ® | C₃F₇O[CF(CF₃)CF₂O]ₓC₂F₅ |
| FOMBLIN ® Y | C₃F₇O[CF(CF₃)CF₂O]ₓ(CF₂O)_{y}C₂F₅ |
| FOMBLIN ® Z | CF₃O(CF₂CF₂O)ₓ(CF₂O)_{y}CF₃ |

TABLE II
PFPE PHYSICAL PROPERTIES

| | Average | Viscosity | | Pour | | |
|---|---|---|---|---|---|---|
| | Molecular | at 20° C., | Viscosity | Point, | Vapor Pressure, Torr | |
| Lubricant | Weight | (cSt) | Index | ° C. | 20° C. | 100° C. |
| FOMBLIN ® | 9500 | 255 | 355 | −66 | 2.9 × 10⁻¹² | 1 × 10⁻⁸ |
| Z-25 | | | | | | |
| KRYTOX ® 143AB | 3700 | 230 | 113 | −40 | 1.5 × 10⁻⁶ | 3 × 10⁻⁴ |
| KRYTOX ® 143AC | 6250 | 800 | 134 | −35 | 2 × 10⁻⁸ | 8 × 10⁻⁶ |
| DEMNUM ® | 8400 | 500 | 210 | −53 | 1 × 10⁻¹⁰ | 1 × 10⁻⁷ |
| S-200 | | | | | | |

TABLE III
PFPE PHYSICAL PROPERTIES OF FUNCTIONAL PFPEs

| | Average | Viscosity | | |
|---|---|---|---|---|
| | Molecular | at 20° C., | Vapor Pressure, Torr | |
| Lubricant | Weight | (cSt) | 20° C. | 100° C. |
| FOMBLIN ® | 2000 | 85 | 2.0 × 10⁻⁵ | 2.0 × 10⁻⁵ |
| Z-DOL 2000 | | | | |
| FOMBLIN ® | 2500 | 76 | 1.0 × 10⁻⁷ | 1.0 × 10⁻⁴ |
| Z-DOL 2500 | | | | |
| FOMBLIN ® | 4000 | 100 | 1.0 × 10⁻⁸ | 1.0 × 10⁻⁴ |
| Z-DOL 4000 | | | | |
| FOMBLIN ® | 500 | 2000 | 5.0 × 10⁻⁷ | 2.0 × 10⁻⁴ |
| Z-TETROL | | | | |

TABLE IV
Names and Chemical Structures of Representative PFPE Fluids

| Name | Structure^{a} |
|---|---|
| Perfluoropoly(methylene oxide) (PMO) | CF₃O(CF₂O)ₓCF₃ |
| Perfluoropoly(ethylene oxide) (PEO) | CF₃O(CF₂CF₂O)ₓCF₃ |
| Perfluoropoly(dioxolane) (DIOX) | CF₃O(CF₂CF₂OCF₂O)ₓCF₃ |
| Perfluoropoly(trioxocane) (TRIOX) | CF₃O[(CF₂CF₂O)₂CF₂O]ₓCF₃ |

^{a}wherein x is any integer.

In some embodiments, the perfluoropolyether precursor includes poly(tetrafluoroethylene oxide-co-difluoromethylene oxide)α,ω diol, which in some embodiments can be photocured to form one of a perfluoropolyether dimethacrylate and a perfluoropolyether distyrenic compound. A representative scheme for the synthesis and photocuring of a functionalized perfluoropolyether is provided in Scheme 1.

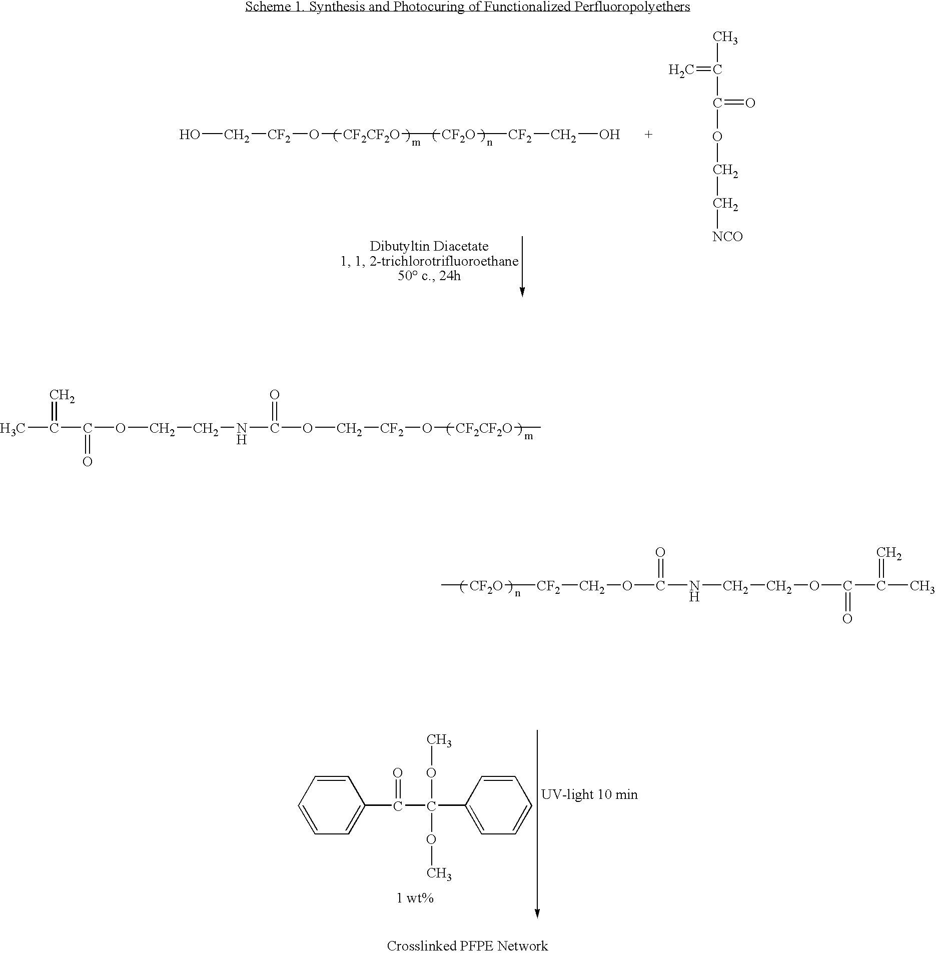

### II.B. Perfluoropolyether Materials Prepared from a Liquid PFPE Precursor Material Having a Viscosity Greater than about 100 Centistokes

The methods provided herein below for promoting and/or increasing adhesion between a layer of a PFPE material and another material and/or a substrate and for adding a chemical functionality to a surface include a PFPE material having a characteristic selected from the group consisting of a viscosity greater than about 100 centistokes (cSt) and a viscosity less than about 100 cSt, provided that the liquid PFPE precursor material having a viscosity less than 100 cSt is not a free-radically photocurable PFPE material. As provided herein, the viscosity of a liquid PFPE precursor material refers to the viscosity of that material prior to functionalization, e.g., functionalization with a methacrylate or a styrenic group.

Thus, in some embodiments, PFPE material is prepared from a liquid PFPE precursor material having a viscosity greater than about 100 centistokes (cSt). In some embodiments, the liquid PFPE precursor is end-capped with a polymerizable group. In some embodiments, the polymerizable group is selected from the group consisting of an acrylate, a methacrylate, an epoxy, an amino, a carboxylic, an anhydride, a maleimide, an isocyanato, an olefinic, and a styrenic group.

In some embodiments, the perfluoropolyether material includes a backbone structure selected from the group consisting of:

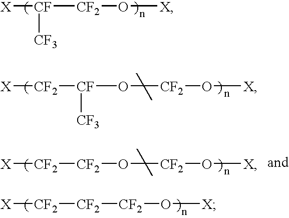

wherein X is present or absent, and when present includes an endcapping group, and n is an integer from 1 to 100.

In some embodiments, the PFPE liquid precursor is synthesized from hexafluoropropylene oxide as shown in Scheme 2.

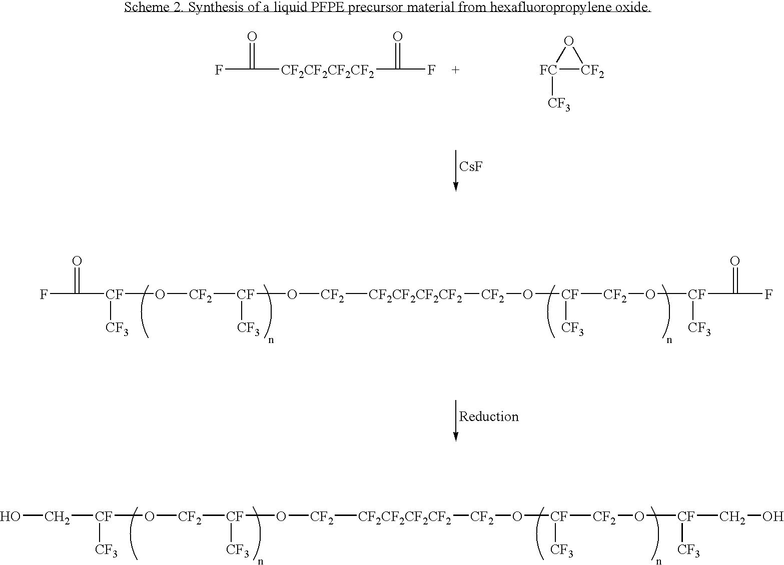

In some embodiments, the liquid PFPE precursor is synthesized from hexafluoropropylene oxide or tetrafluoro ethylene oxide as shown in Scheme 3A or 3B.

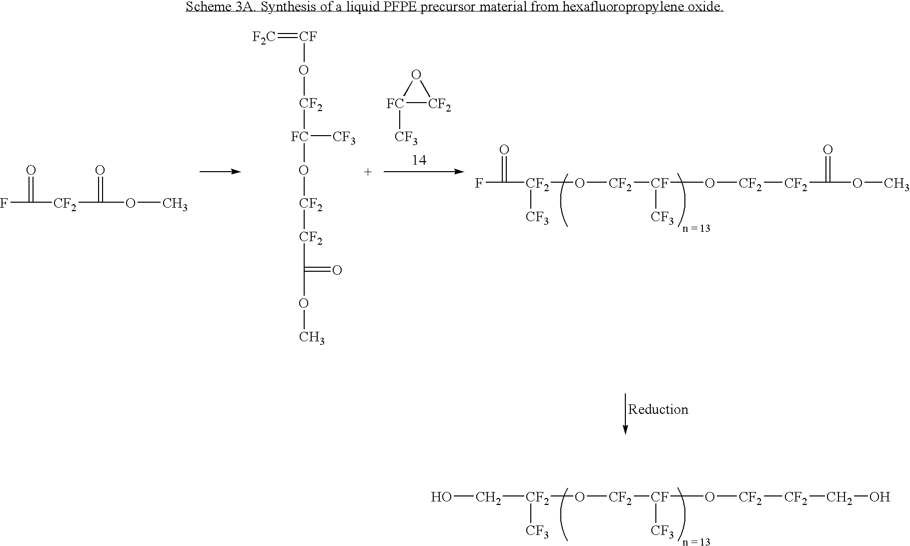

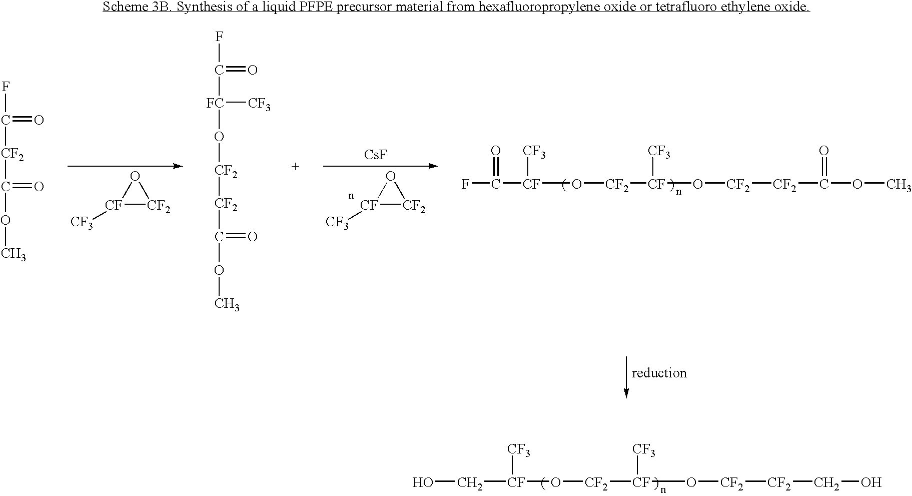

In some embodiments the liquid PFPE precursor includes a chain extended material such that two or more chains are linked together before adding polymerizablable groups. Accordingly, in some embodiments, a “linker group” joins two chains to one molecule. In some embodiments, as shown in Scheme 4, the linker group joins three or more chains.

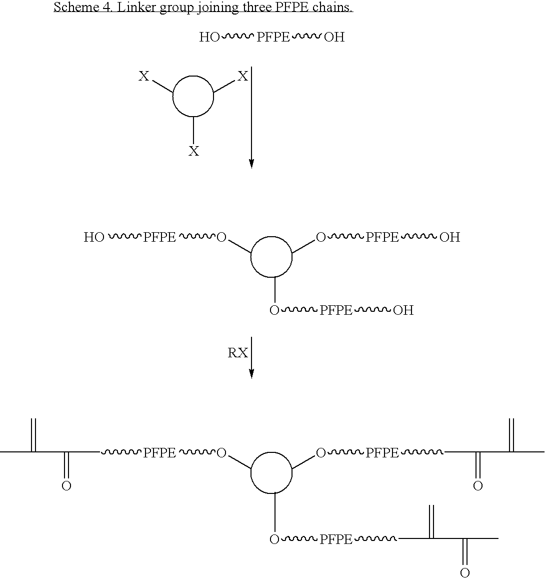

In some embodiments, X is selected from the group consisting of an isocyanate, an acid chloride, an epoxy, and a halogen. In some embodiments, R is selected from the group consisting of an acrylate, a methacrylate, a styrene, an epoxy, a carboxylic, an anhydride, a maleimide, an isocyanate, an olefinic, and an amine. In some embodiments, the circle represents any multifunctional molecule. In some embodiments, the multifunctional molecule includes a cyclic molecule. PFPE refers to any PFPE material provided hereinabove.

In some embodiments, the liquid PFPE precursor includes a hyperbranched polymer as provided in Scheme 5, wherein PFPE refers to any PFPE material provided hereinabove.

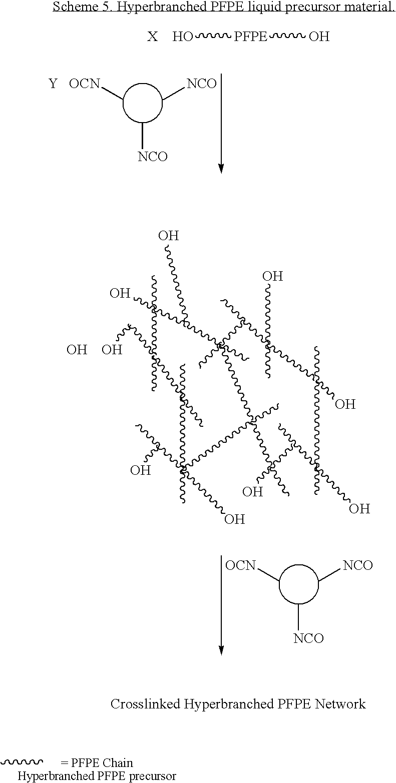

In some embodiments, the liquid PFPE material includes an end-functionalized material selected from the group consisting of:

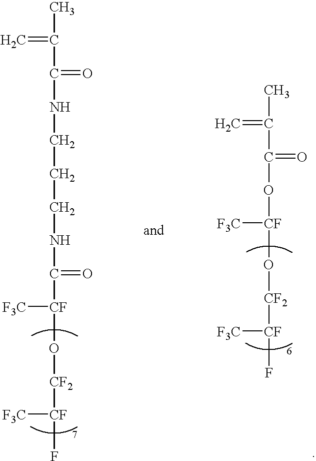

In some embodiments the PFPE liquid precursor is encapped with an epoxy moiety that can be photocured using a photoacid generator. Photoacid generators suitable for use in the presently disclosed subject matter include, but are not limited to: bis(4-tert-butylphenyl)iodonium p-toluenesulfonate, bis(4-tert-butylphenyl)iodonium triflate, (4-bromophenyl)diphenylsulfonium triflate, (tert-butoxycarbonylmethoxynaphthyl)-diphenylsulfonium triflate, (tert-butoxycarbonylmethoxyphenyl)diphenylsulfonium triflate, (4-tert-butylphenyl)diphenylsulfonium triflate, (4-chlorophenyl)diphenylsulfonium triflate, diphenyliodonium-9,10-dimethoxyanthracene-2-sulfonate, diphenyliodonium hexafluorophosphate, diphenyliodonium nitrate, diphenyliodonium perfluoro-1-butanesulfonate, diphenyliodonium p-toluenesulfonate, diphenyliodonium triflate, (4-fluorophenyl)diphenylsulfonium triflate, N-hydroxynaphthalimide triflate, N-hydroxy-5-norbornene-2,3-dicarboximide perfluoro-1-butanesulfonate, N-hydroxyphthalimide triflate, [4-[(2-hydroxytetradecyl)oxy]phenyl]phenyliodonium hexafluoroantimonate, (4-iodophenyl)diphenylsulfonium triflate, (4-methoxyphenyl)diphenylsulfonium triflate, 2-(4-methoxystyryl)-4,6-bis(trichloromethyl)-1,3,5-triazine, (4-methylphenyl)diphenylsulfonium triflate, (4-methylthiophenyl)methyl phenyl sulfonium triflate, 2-naphthyl diphenylsulfonium triflate, (4-phenoxyphenyl)diphenylsulfonium triflate, (4-phenylthiophenyl)diphenylsulfonium triflate, thiobis(triphenyl sulfonium hexafluorophosphate), triarylsulfonium hexafluoroantimonate salts, triarylsulfonium hexafluorophosphate salts, triphenylsulfonium perfluoro-1-butanesufonate, triphenylsulfonium triflate, tris(4-tert-butylphenyl)sulfonium perfluoro-1-butanesulfonate, and tris(4-tert-butylphenyl)sulfonium triflate.

In some embodiments the liquid PFPE precursor cures into a highly UV and/or highly visible light transparent elastomer. In some embodiments the liquid PFPE precursor cures into an elastomer that is highly permeable to oxygen, carbon dioxide, and nitrogen, a property that can facilitate maintaining the viability of biological fluids/cells disposed therein. In some embodiments, additives are added or layers are created to enhance the barrier properties of the device to molecules, such as oxygen, carbon dioxide, nitrogen, dyes, reagents, and the like.

In some embodiments, the material suitable for use with the presently disclosed subject matter includes a silicone material having a fluoroalkyl functionalized polydimethylsiloxane (PDMS) having the following structure:

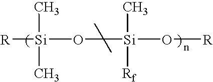

wherein:

R is selected from the group consisting of an acrylate, a methacrylate, and a vinyl group;

R_{f} includes a fluoroalkyl chain; and

n is an integer from 1 to 100,000.

In some embodiments, the material suitable for use with the presently disclosed subject matter includes a styrenic material having a fluorinated styrene monomer selected from the group consisting of:

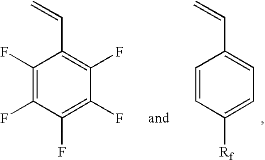

wherein R_{f} includes a fluoroalkyl chain.

In some embodiments, the material suitable for use with the presently disclosed subject matter includes an acrylate material having a fluorinated acrylate or a fluorinated methacrylate having the following structure:

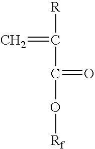

wherein:

R is selected from the group consisting of H, alkyl, substituted alkyl, aryl, and substituted aryl; and

R_{f} includes a fluoroalkyl chain with a —CH₂— or a —CH₂—CH₂— spacer between a perfluoroalkyl chain and the ester linkage. In some embodiments, the perfluoroalkyl group has hydrogen substituents.

In some embodiments, the material suitable for use with the presently disclosed subject matter includes a triazine fluoropolymer having a fluorinated monomer.

In some embodiments, the fluorinated monomer or fluorinated oligomer that can be polymerized or crosslinked by a metathesis polymerization reaction includes a functionalized olefin. In some embodiments, the functionalized olefin includes a functionalized cyclic olefin.

According to an alternative embodiment, the PFPE material includes a urethane block as described and shown in the following structures provided in Scheme 6:

PFPE Urethane Tetrafunctional Methacrylate

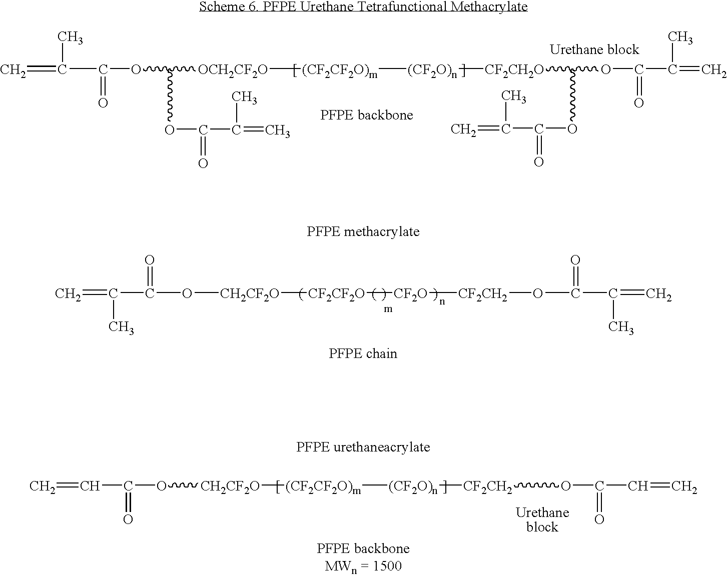

According to an embodiment of the present invention, PFPE urethane tetrafunctional methacrylate materials such as the above described can be used as the materials and methods of the present invention or can be used in combination with other materials and methods described herein, as will be appreciated by one of ordinary skill in the art.

### II.C. Fluoroolefin-Based Materials

Further, in some embodiments, the materials used herein are selected from highly fluorinated fluoroelastomers, e.g., fluoroelastomers having at least fifty-eight weight percent fluorine, as described in U.S. Pat. No. 6,512,063 to Tang, which is incorporated herein by reference in its entirety. Such fluoroelastomers can be partially fluorinated or perfluorinated and can contain between about 25 to about 70 weight percent, based on the weight of the fluoroelastomer, of copolymerized units of a first monomer, e.g., vinylidene fluoride (VF₂) or tetrafluoroethylene (TFE). The remaining units of the fluoroelastomers include one or more additional copolymerized monomers, which are different from the first monomer, and are selected from the group consisting of fluorine-containing olefins, fluorine containing vinyl ethers, hydrocarbon olefins, and combinations thereof.

These fluoroelastomers include VITON® (DuPont Dow Elastomers, Wilmington, Del., United States of America) and Kel-F type polymers, as described for microfluidic applications in U.S. Pat. No. 6,408,878 to Unger et al. These commercially available polymers, however, have Mooney viscosities ranging from about 40 to about 65 (ML 1+10 at 121° C.) giving them a tacky, gum-like viscosity. When cured, they become a stiff, opaque solid. As currently available, VITON® and Kel-F have limited utility for micro-scale molding. Curable species of similar compositions, but having lower viscosity and greater optical clarity, is needed in the art for the applications described herein. A lower viscosity (e.g., about 2 to about 32 (ML 1+10 at 121° C.)) or more preferably as low as about 80 to about 2000 cSt at 20 C, composition yields a pourable liquid with a more efficient cure.

More particularly, the fluorine-containing olefins include, but are not limited to, vinylidine fluoride, hexafluoropropylene (HFP), tetrafluoroethylene (TFE), 1,2,3,3,3-pentafluoropropene (1-HPFP), chlorotrifluoroethylene (CTFE) and vinyl fluoride.

The fluorine-containing vinyl ethers include, but are not limited to perfluoro(alkyl vinyl) ethers (PAVEs). More particularly, perfluoro(alkyl vinyl) ethers for use as monomers include perfluoro(alkyl vinyl) ethers of the following formula:
CF₂═CFO(R_{f}O)ₙ(R_{f}O)ₘR_{f}

wherein each R_{f} is independently a linear or branched C₁-C₆ perfluoroalkylene group, and m and n are each independently an integer from 0 to 10.

In some embodiments, the perfluoro(alkyl vinyl) ether includes a monomer of the following formula:
CF₂═CFO(CF₂CFXO)ₙR_{f}

wherein X is F or CF₃, n is an integer from 0 to 5, and R_{f} is a linear or branched C₁-C₆ perfluoroalkylene group. In some embodiments, n is 0 or 1 and R_{f} includes 1 to 3 carbon atoms. Representative examples of such perfluoro(alkyl vinyl) ethers include perfluoro(methyl vinyl) ether (PMVE) and perfluoro(propyl vinyl) ether (PPVE).

In some embodiments, the perfluoro(alkyl vinyl) ether includes a monomer of the following formula:
CF₂═CFO[(CF₂)ₘCF₂CFZO)ₙR_{f}

wherein R_{f} is a perfluoroalkyl group having 1-6 carbon atoms, m is an integer from 0 or 1, n is an integer from 0 to 5, and Z is F or CF₃. In some embodiments, R_{f} is C₃F₇, m is 0, and n is 1.

In some embodiments, the perfluoro(alkyl vinyl) ether monomers include compounds of the formula:
CF₂═CFO[(CF₂CF{CF₃}O)ₙ(CF₂CF₂CF₂O)ₘ(CF2)ₚ]CₓF₂ₓ₊₁

wherein m and n each integers independently from 0 to 10, p is an integer from 0 to 3, and x is an integer from 1 to 5. In some embodiments, n is 0 or 1, m is 0 or 1, and x is 1.

Other examples of useful perfluoro(alkyl vinyl ethers) include:
CF₂═CFOCF₂CF(CF₃)O(CF₂O)ₘCₙF₂ₙ₊₁

wherein n is an integer from 1 to 5, m is an integer from 1 to 3. In some embodiments, n is 1.

In embodiments where copolymerized units of a perfluoro(alkyl vinyl) ether (PAVE) are present in the presently described fluoroelastomers, the PAVE content generally ranges from about 25 to about 75 weight percent, based on the total weight of the fluoroelastomer. If the PAVE is perfluoro(methyl vinyl) ether (PMVE), then the fluoroelastomer contains between about 30 and about 55 wt. % copolymerized PMVE units.

Hydrocarbon olefins useful in the presently described fluoroelastomers include, but are not limited to ethylene (E) and propylene (P). In embodiments wherein copolymerized units of a hydrocarbon olefin are present in the presently described fluoroelastomers, the hydrocarbon olefin content is generally about 4 to about 30 weight percent.

Further, in some embodiments the fluoroelastomers can include units of one or more cure site monomers. Examples of suitable cure site monomers include: i) bromine-containing olefins; ii) iodine-containing olefins; iii) bromine-containing vinyl ethers; iv) iodine-containing vinyl ethers; v) fluorine-containing olefins having a nitrile group; vi) fluorine-containing vinyl ethers having a nitrile group; vii) 1,1,3,3,3-pentafluoropropene (2-HPFP); viii) perfluoro(2-phenoxypropyl vinyl) ether; and ix) non-conjugated dienes.

The brominated cure site monomers can contain other halogens, preferably fluorine. Examples of brominated olefin cure site monomers are CF₂═CFOCF₂CF₂CF₂OCF₂CF₂Br; bromotrifluoroethylene; 4-bromo-3,3,4,4-tetrafluorobutene-1 (BTFB); and others such as vinyl bromide, 1-bromo-2,2-difluoroethylene; perfluoroallyl bromide; 4-bromo-1,1,2-trifluorobutene-1; 4-bromo-1,1,3,3,4,4,-hexafluorobutene; 4-bromo-3-chloro-1,1,3,4,4-pentafluorobutene; 6-bromo-5,5,6,6-tetrafluorohexene; 4-bromoperfluorobutene-1 and 3,3-difluoroallyl bromide. Brominated vinyl ether cure site monomers include 2-bromo-perfluoroethyl perfluorovinyl ether and fluorinated compounds of the class CF₂Br—R_{f}—O—CF═CF₂ (wherein R_{f} is a perfluoroalkylene group), such as CF₂BrCF₂O—CF═CF₂, and fluorovinyl ethers of the class ROCF═CFBr or ROCBr═CF₂ (wherein R is a lower alkyl group or fluoroalkyl group), such as CH₃OCF═CFBr or CF₃CH₂OCF═CFBr.

Suitable iodinated cure site monomers include iodinated olefins of the formula: CHR═CH-Z-CH₂CHR—I, wherein R is —H or —CH₃; Z is a C₁ to C₁₈ (per)fluoroalkylene radical, linear or branched, optionally containing one or more ether oxygen atoms, or a (per)fluoropolyoxyalkylene radical as disclosed in U.S. Pat. No. 5,674,959. Other examples of useful iodinated cure site monomers are unsaturated ethers of the formula: I(CH₂CF₂CF₂)ₙOCF═CF₂ and ICH₂CF₂O[CF(CF₃)CF₂O]ₙCF═CF₂, and the like, wherein n is an integer from 1 to 3, such as disclosed in U.S. Pat. No. 5,717,036. In addition, suitable iodinated cure site monomers including iodoethylene, 4-iodo-3,3,4,4-tetrafluorobutene-1 (ITFB); 3-chloro-4-iodo-3,4,4-trifluorobutene; 2-iodo-1,1,2,2-tetrafluoro-1-(vinyloxy)ethane; 2-iodo-1-(perfluorovinyloxy)-1,1,-2,2-tetrafluoroethylene; 1,1,2,3,3,3-hexafluoro-2-iodo-1-(perfluorovinyloxy)propane; 2-iodoethyl vinyl ether; 3,3,4,5,5,5-hexafluoro-4-iodopentene; and iodotrifluoroethylene are disclosed in U.S. Pat. No. 4,694,045. Allyl iodide and 2-iodo-perfluoroethyl perfluorovinyl ether also are useful cure site monomers.

Useful nitrile-containing cure site monomers include those of the formulas shown below:
CF₂═CF—O(CF₂)ₙ—CN

wherein n is an integer from 2 to 12. In some embodiments, n is an integer from 2 to 6.
CF₂═CF—O[CF₂—CF(CF)—O]ₙ—CF₂—CF(CF₃)—CN

wherein n is an integer from 0 to 4. In some embodiments, n is an integer from 0 to 2.
CF₂═CF—[OCF₂CF(CF₃)]ₓ—O—(CF₂)ₙ—CN

wherein x is 1 or 2, and n is an integer from 1 to 4; and
CF₂═CF—O—(CF₂)ₙ—O—CF(CF₃)—CN

wherein n is an integer from 2 to 4. In some embodiments, the cure site monomers are perfluorinated polyethers having a nitrile group and a trifluorovinyl ether group.

In some embodiments, the cure site monomer is:
CF₂═CFOCF₂CF(CF₃)OCF₂CF₂CN

i.e., perfluoro(8-cyano-5-methyl-3,6-dioxa-1-octene) or 8-CNVE.

Examples of non-conjugated diene cure site monomers include, but are not limited to 1,4-pentadiene; 1,5-hexadiene; 1,7-octadiene; 3,3,4,4-tetrafluoro-1,5-hexadiene; and others, such as those disclosed in Canadian Patent No. 2,067,891 and European Patent No. 0784064A1. In some embodiments, a suitable triene is 8-methyl-4-ethylidene-1,7-octadiene.

In embodiments where the fluoroelastomer will be cured with peroxide, the cure site monomer is preferably selected from the group consisting of 4-bromo-3,3,4,4-tetrafluorobutene-1 (BTFB); 4-iodo-3,3,4,4-tetrafluorobutene-1 (ITFB); allyl iodide; bromotrifluoroethylene and 8-CNVE. In embodiments wherein the fluoroelastomer will be cured with a polyol, 2-HPFP or perfluoro(2-phenoxypropyl vinyl) ether is the preferred cure site monomer. In embodiments wherein the fluoroelastomer will be cured with a tetraamine, bis(aminophenol) or bis(thioaminophenol), 8-CNVE is the preferred cure site monomer.

Units of cure site monomer, when present in the fluoroelastomers, are typically present at a level of about 0.05 wt. % to about 10 wt. % (based on the total weight of fluoroelastomer), preferably about 0.05 wt. % to about 5 wt. % and more preferably between about 0.05 wt. % and about 3 wt. %.

Fluoroelastomers which can be used in the presently disclosed subject matter include, but are not limited to, those having at least about 58 wt. % fluorine and having copolymerized units of i) vinylidene fluoride and hexafluoropropylene; ii) vinylidene fluoride, hexafluoropropylene and tetrafluoroethylene; iii) vinylidene fluoride, hexafluoropropylene, tetrafluoroethylene and 4-bromo-3,3,4,4-tetrafluorobutene-1; iv) vinylidene fluoride, hexafluoropropylene, tetrafluoroethylene and 4-iodo-3,3,4,4-tetrafluorobutene-1; v) vinylidene fluoride, perfluoro(methyl vinyl) ether, tetrafluoroethylene and 4-bromo-3,3,4,4-tetrafluorobutene-1; vi) vinylidene fluoride, perfluoro(methyl vinyl) ether, tetrafluoroethylene and 4-iodo-3,3,4,4-tetrafluorobutene-1; vii) vinylidene fluoride, perfluoro(methyl vinyl) ether, tetrafluoroethylene and 1,1,3,3,3-pentafluoropropene; viii) tetrafluoroethylene, perfluoro(methyl vinyl) ether and ethylene; ix) tetrafluoroethylene, perfluoro(methyl vinyl) ether, ethylene and 4-bromo-3,3,4,4-tetrafluorobutene-1; x) tetrafluoroethylene, perfluoro(methyl vinyl) ether, ethylene and 4-iodo-3,3,4,4-tetrafluorobutene-1; xi) tetrafluoroethylene, propylene and vinylidene fluoride; xii) tetrafluoroethylene and perfluoro(methyl vinyl) ether; xiii) tetrafluoroethylene, perfluoro(methyl vinyl) ether and perfluoro(8-cyano-5-methyl-3,6-dioxa-1-octene); xiv) tetrafluoroethylene, perfluoro(methyl vinyl) ether and 4-bromo-3,3,4,4-tetrafluorobutene-1; xv) tetrafluoroethylene, perfluoro(methyl vinyl) ether and 4-iodo-3,3,4,4-tetrafluorobutene-1; and xvi) tetrafluoroethylene, perfluoro(methyl vinyl) ether and perfluoro(2-phenoxypropyl vinyl) ether.

Additionally, iodine-containing endgroups, bromine-containing endgroups or combinations thereof can optionally be present at one or both of the fluoroelastomer polymer chain ends as a result of the use of chain transfer or molecular weight regulating agents during preparation of the fluoroelastomers. The amount of chain transfer agent, when employed, is calculated to result in an iodine or bromine level in the fluoroelastomer in the range of about 0.005 wt. % to about 5 wt. %, and preferably about 0.05 wt. % to about 3 wt. %.

Examples of chain transfer agents include iodine-containing compounds that result in incorporation of bound iodine at one or both ends of the polymer molecules. Methylene iodide; 1,4-diiodoperfluoro-n-butane; and 1,6-diiodo-3,3,4,4-tetrafluorohexane are representative of such agents. Other iodinated chain transfer agents include 1,3-diiodoperfluoropropane; 1,6-diiodoperfluorohexane; 1,3-diiodo-2-chloroperfluoropropane; 1,2-di(iododifluoromethyl)perfluorocyclobutane; monoiodoperfluoroethane; monoiodoperfluorobutane; 2-iodo-1-hydroperfluoroethane, and the like. Also included are the cyano-iodine chain transfer agents disclosed European Patent No. 0868447A1. Particularly preferred are diiodinated chain transfer agents.

Examples of brominated chain transfer agents include 1-bromo-2-iodoperfluoroethane; 1-bromo-3-iodoperfluoropropane; 1-iodo-2-bromo-1,1-difluoroethane and others such as disclosed in U.S. Pat. No. 5,151,492.

Other chain transfer agents suitable for use include those disclosed in U.S. Pat. No. 3,707,529. Examples of such agents include isopropanol, diethylmalonate, ethyl acetate, carbon tetrachloride, acetone and dodecyl mercaptan.

### II.D. Dual Photo-Curable and Thermal-Curable Materials

According to another embodiment, a material according to the invention includes one or more of a photo-curable constituent and a thermal-curable constituent. In one embodiment, the photo-curable constituent is independent from the thermal-curable constituent such that the material can undergo multiple cures. A material having the ability to undergo multiple cures is useful, for example, in forming layered devices or in connecting or attaching devices to other devices or portions or components of devices to other portions or components of devices. For example, a liquid material having photocurable and thermal-curable constituents can undergo a first cure to form a first device through, for example, a photocuring process or a thermal curing process. Then the photocured or thermal cured first device can be adhered to a second device of the same material or any material similar thereto that will thermally cure or photocure and bind to the material of the first device. By positioning the first device and second device adjacent one another and subjecting the first and second devices to a thermalcuring or photocuring, whichever component that was not activated on the first curing. Thereafter, either the thermalcure constituents of the first device that were left un-activated by the photocuring process or the photocure constituents of the first device that were left un-activated by the first thermal curing, will be activated and bind the second device. Thereby, the first and second devices become adhered together. It will be appreciated by one of ordinary skill in the art that the order of curing processes is independent and a thermal-curing could occur first followed by a photocuring or a photocuring could occur first followed by a thermal curing.

According to yet another embodiment, multiple thermo-curable constituents can be included in the material such that the material can be subjected to multiple independent thermal-cures. For example, the multiple thermo-curable constituents can have different activation temperature ranges such that the material can undergo a first thermal-cure at a first temperature range and a second thermal-cure at a second temperature range. Accordingly, the material can be adhered to multiple other materials through different thermal-cures, thereby, forming a multiple laminate layer device.

Examples of chemical groups which would be suitable end-capping agents for a UV curable component include: methacrylates, acrylates, styrenics, epoxides, cyclobutanes and other 2+2 cycloadditions, combinations thereof, and the like. Examples of chemical group pairs which are suitable to endcap a thermally curable component include: epoxy/amine, epoxy/hydroxyl, carboxylic acid/amine, carboxylic acid/hydroxyl, ester/amine, ester/hydroxyl, amine/anhydride, acid halide/hydroxyl, acid halide/amine, amine/halide, hydroxyl/halide, hydroxyvchlorosilane, azide/acetylene and other so-called “click chemistry” reactions, and metathesis reactions involving the use of Grubb's-type catalysts, combinations thereof, and the like.

The methods of adhesion of multiple layers of one device to another or to a separate surface can be applied to PFPE-based materials, as described herein, as well as a variety of other materials, including PDMS and other liquid-like polymers. Examples of liquid-like polymeric materials that are suitable for use in the presently disclosed adhesion methods include, but are not limited to, PDMS, poly(tetramethylene oxide), poly(ethylene oxide), poly(oxetanes), polyisoprene, polybutadiene, and fluoroolefin-based fluoroelastomers, such as those available under the registered trademarks VITON® AND KALREZ®.

Accordingly, layers of different polymeric materials can be adhered together to form devices, such as medical device, surgical devices, tools, components of medical devices, implant materials, implantable articles, medical articles, laminates, combinations thereof, and the like (collectively “medical devices”).

### II.E. Silicone Based Materials

According to alternate embodiments, novel silicone based materials include photocurable and thermal-curable components. In such alternate embodiments, silicone based materials can include one or more photo-curable and thermal-curable components such that the silicone based material has a dual curing capability as described herein. Silicone based materials compatible with the present invention are described herein and throughout the reference materials incorporated by reference into this application.

III. Medical or Surgical Device Formed Through a Thermal Free Radical Curing Process

In some embodiments, a medical or surgical device is formed from a polymeric material utilizing a thermal free radical curing process. Such medical and surgical devices can be formed by contacting a functional liquid perfluoropolyether (PFPE) precursor material with a patterned substrate, i.e., a master, and is thermally cured while in contact with the patterned substrate using a free radical initiator. As provided in more detail herein below, in some embodiments, the liquid PFPE precursor material is fully cured to form a fully cured PFPE network, which can then be removed from the patterned substrate and contacted with a second substrate to form a reversible, hermetic seal.

In some embodiments, the liquid PFPE precursor material is partially cured to form a partially cured PFPE network. In some embodiments, the partially cured network is contacted with a second partially cured layer of PFPE material and the curing reaction is taken to completion, thereby forming a bond between the PFPE layers.

Further, the partially cured PFPE network can be contacted with a layer or substrate including another polymeric material, such as poly(dimethylsiloxane) or another polymer, and then thermally cured so that the PFPE network adheres to the other polymeric material. Additionally, the partially cured PFPE network can be contacted with a solid substrate, such as glass, quartz, or silicon, and then bonded to the substrate through the use of a silane coupling agent.

### III.A. A Patterned Layer Formed of an Elastomeric Material

In some embodiments, a patterned layer of an elastomeric material is formed. The presently disclosed method is suitable for use with, among other materials, the perfluoropolyether material described in Sections II.A. and II.B., and the fluoroolefin-based materials described in Section II.C. An advantage of using a higher viscosity PFPE material allows, among other things, for a higher molecular weight between cross links. A higher molecular weight between cross links can improve the elastomeric properties of the material, which can prevent among other things, cracks from forming.

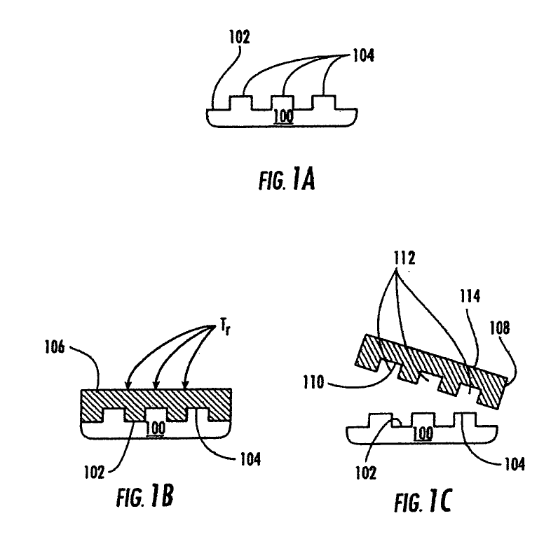

Referring now to FIGS. 1A-1C, a substrate 100 has a patterned surface 102 with a raised protrusion 104. Accordingly, the patterned surface 102 of the substrate 100 includes at least one raised protrusion 104, which forms the shape of a pattern. In some embodiments, patterned surface 102 of substrate 100 includes a plurality of raised protrusions 104 which form a complex pattern.

As best seen in FIG. 1B, a liquid precursor material 106 is disposed on patterned surface 102 of substrate 100. As shown in FIG. 1B, the liquid precursor material 102 is treated with a treating process Tᵣ. Upon the treating of liquid precursor material 106, a patterned layer 108 of an elastomeric material (as shown in FIG. 1C) is formed.

As shown in FIG. 1C, the patterned layer 108 of the elastomeric material includes a recess 110 that is formed in the bottom surface of the patterned layer 108. The dimensions of recess 110 correspond to the dimensions of the raised protrusion 104 of patterned surface 102 of substrate 100. In some embodiments, recess 110 includes at least one channel 112, which in some embodiments of the presently disclosed subject matter includes a microscale channel or groove. Patterned layer 108 is removed from patterned surface 102 of substrate 100 to yield device 114. In some embodiments, removal of device 114 is performed using a “lift-off” solvent which slowly wets underneath the device and releases it from the patterned substrate. Examples of such solvents include, but are not limited to, any solvent that will not adversely interact with the material of the patterned layer 108 or functional components of the patterned layer 108. Examples of such solvents include vary depending on what polymer is utilized in fabricating the patterned layer 108 and include, but are not limited to: water, isopropyl alcohol, acetone, N-methylpyrollidinone, dimethyl formamide, combinations thereof, and the like.

In some embodiments, the patterned substrate includes a structure on an etched silicon wafer. In some embodiments, the patterned substrate includes a photoresist patterned substrate. In some embodiments, the patterned substrate is treated with a coating that can aid in the release of the device from the patterned substrate or prevent reaction with latent groups on a photoresist which constitutes the patterned substrate. An example of the coating can include, but is not limited to, a silane or thin film of metal deposited from a plasma, such as, a Gold/Palladium coating. For the purposes of the present disclosure, the patterned substrate can be fabricated by any of the processing methods known in the art, including, but not limited to, photolithography, electron beam lithography, ion milling, combinations thereof, and the like.

In some embodiments, the patterned layer of perfluoropolyether is between about 0.1 micrometers and about 100 micrometers thick. In some embodiments, the patterned layer of perfluoropolyether is between about 0.1 millimeters and about 10 millimeters thick. In some embodiments, the patterned layer of perfluoropolyether is between about one micrometer and about 50 micrometers thick. In some embodiments, the patterned layer of perfluoropolyether is about 20 micrometers thick. In other embodiments, the patterned layer of perfluoropolyether is about 5 millimeters thick.

In some embodiments, the patterned structures of the perfluoropolyether layer includes a plurality of microscale grooves, or structures. In some embodiments, the grooves or structures have a width ranging from about 0.01 μm to about 1000 μm. In other embodiments the plurality of structures has a width ranging from about 0.05 μm to about 1000 μm. In yet other embodiments the plurality of structures has a width ranging from about 1 μm to about 1000 μm. In still other embodiments, the structures have a width ranging from about 1 μm to about 500 μm. In other embodiments the plurality of structures has a width ranging from about 1 μm to about 250 μm. In still further embodiments the plurality of structures include a width ranging from about 10 μm to about 200 μm. Exemplary groove, or structure widths include, but are not limited to, 0.1 μm, 1 μm, 2 μm, 5 μm, 10 μm, 20 μm, 30 μm, 40 μm, 50 μm, 60 μm, 70 μm, 80 μm, 90 μm, 100 μm, 110 μm, 120 μm, 130 μm, 140 μm, 150 μm, 160 μm, 170 μm, 180 μm, 190 μm, 200 μm, 210 μm, 220 μm, 230 μm, 240 μm, 250 μm, combinations thereof, and the like.

In some embodiments, the microscale grooves, or structures of the patterned perfluoropolyether layer have a depth ranging from about 1 μm to about 1000 μm. According to other embodiments the plurality of structures has a depth ranging from about 1 μm to 100 μm. In some embodiments, the structures have a depth ranging from about 0.01 μm to about 1000 μm. In other embodiments the plurality of structures has a depth ranging from about 0.05 μm to about 500 μm. In yet other embodiments the plurality of structures has a depth ranging from about 0.2 μm to about 250 μm. In still further embodiments the plurality of structures include a depth ranging from about 1 μm to about 100 μm. In other embodiments the plurality of structures has a depth ranging from about 2 μm to about 20 μm. In other embodiments the plurality of structures has a depth ranging from about 5 μm to about 10 μm. Exemplary channel depths include, but are not limited to, 0.01 μm, 0.02 μm, 0.05 μm, 0.1 μm, 0.2 μm, 0.5 μm, 1 μm, 2 μm, 3 μm, 4 μm, 5 μm, 7.5 μm, 10 μm, 12.5 μm, 15 μm, 17.5 μm, 20 μm, 22.5 μm, 25 μm, 30 μm, 40 μm, 50 μm, 75 μm, 100 μm, 150 μm, 200 μm, 250 μm, combinations thereof, and the like.

In some embodiments, the structures have a width-to-depth ratio ranging from about 0.1:1 to about 100:1. In some embodiments, the structures have a width-to-depth ratio ranging from about 1:1 to about 50:1. In some embodiments, the structures have a width-to-depth ratio ranging from about 2:1 to about 20:1. In some embodiments, the structures have a width-to-depth ratio ranging from about 3:1 to about 15:1. In some embodiments, the structures have a width-to-depth ratio of about 10:1.

It should be appreciated that the dimensions of the structures are not limited to the exemplary dimensions and ranges described hereinabove and can vary in width, depth, and ratio to affect the desired outcome such as a magnitude of force required to flow a material through the channel, promote or inhibit adhesion to a surface by a bio-molecule or infectious agent, or the like.

### III.B. A Multilayer Patterned Device

In some embodiments, a multilayer patterned material is formed, such as a multilayer patterned PFPE material and applied to, used in connection with, or used as a medical or surgical device. In some embodiments, the multilayer patterned perfluoropolyether material is used to fabricate a monolithic PFPE-based medical or surgical device.

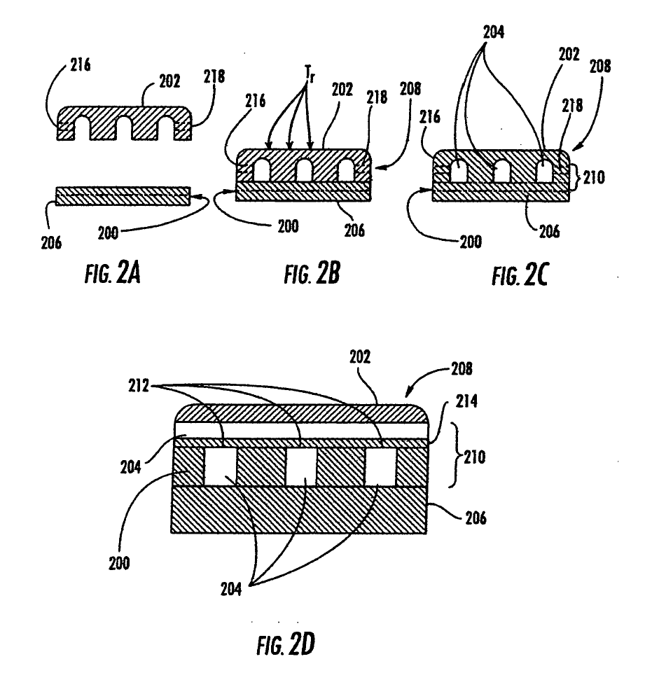

Referring now to FIGS. 2A-2D, patterned layers 200 and 202 are provided, each of which, in some embodiments, include or consist entirely of a perfluoropolyether material prepared from a liquid PFPE precursor material having a viscosity greater than about 100 cSt. In this example, each of the patterned layers 200 and 202 include a plurality of channels or structures 204. Also, in this embodiment the plurality of c structures 204 include microscale structures or channels. In patterned layer 200, the structures are represented by a dashed line, i.e., in shadow, in FIGS. 2A-2C. Patterned layer 202 is overlaid on patterned layer 200 in a predetermined alignment. In this example, the predetermined alignment is such that structures 204 in patterned layer 200 and 202 are substantially perpendicular to each other. In some embodiments, as depicted in FIGS. 2A-2D, patterned layer 200 is overlaid on nonpatterned layer 206. Nonpatterned layer 206 can include a perfluoropolyether.

Continuing with reference to FIGS. 2A-2D, patterned layers 200 and 202, and in some embodiments nonpatterned layer 206, are treated by a treating process Tᵣ. As described in more detail herein, layers 200, 202, and, in some embodiments nonpatterned layer 206, are treated by treating Tᵣ, to promote the adhesion of patterned layers 200 and 202 to each other, and in some embodiments, patterned layer 200 to nonpatterned layer 206, as shown in FIGS. 2C and 2D. The resulting device 208 includes an integrated network 210 of microscale structures 204 that can intersect at predetermined intersecting points 212, as best seen in the cross-section provided in FIG. 2D. Also shown in FIG. 2D is membrane 214 including the top surface of structures 204 of patterned layer 200 which separates structures 204 of patterned layer 202 from structures 204 of patterned layer 200.

Continuing with reference to FIGS. 2A-2C, in some embodiments, patterned layer 202 includes a plurality of apertures, and the apertures are designated input aperture 216 and output aperture 218. In some embodiments, the holes, e.g., input aperture 216 and output aperture 218 are in fluid communication with channels 204. In some embodiments, the apertures include a side-actuated valve structure constructed of, for example, a thin membrane of PFPE material which can be actuated to restrict the flow in an abutting channel. It will be appreciated, however, that the side-actuated valve structure can be constructed from other materials disclosed herein.

In some embodiments, the first patterned layer of material is cast at such a thickness to impart a degree of mechanical stability to the resulting structure. Accordingly, in some embodiments, the first patterned layer of material can be about 50 μm to several centimeters thick. In some embodiments, the first patterned layer of material is between 50 μm and about 10 millimeters thick. In some embodiments, the first patterned layer of the material is about 5 mm thick. In some embodiments, the first patterned layer of material is about 4 mm thick. Further, in alternative embodiments, the thickness of the first patterned layer of material ranges from about 0.1 μm to about 10 cm; from about 1 μm to about 5 cm; from about 10 μm to about 2 cm; or from about 100 μm to about 10 mm thick, respectively. In some embodiments, the material is PFPE or another polymeric material disclosed herein.

In some embodiments, the second patterned layer of the material is between about 1 micrometer and about 100 micrometers thick. In some embodiments, the second patterned layer of material is between about 1 micrometer and about 50 micrometers thick. In some embodiments, the second patterned layer of material is about 20 micrometers thick. In some embodiments, the material is PFPE or another polymeric material disclosed herein.

Although FIGS. 2A-2C disclose the formation of a device by combining two patterned layers of material, in some embodiments a device is formed that has one patterned layer and one non-patterned layer of material. Thus, the first patterned layer can include a structure or an integrated network of structures and then the first patterned layer can be overlaid on top of a non-patterned layer and adhered to the non-patterned layer using a photocuring step, such as through the application of ultraviolet light as disclosed herein, or by using a thermal curing step also as disclosed herein, to form a monolithic device including enclosed structures therein. In some embodiments, the material is PFPE or another polymeric material disclosed herein.

### III.C. A Patterned PFPE Layer Through a Thermal Free Radical Curing Process

In some embodiments, a thermal free radical initiator, including, but not limited to, a peroxide and/or an azo compound, is blended with a liquid perfluoropolyether (PFPE) precursor, which is functionalized with a polymerizable group, including, but not limited to, an acrylate, a methacrylate, and a styrenic unit to form a blend. As shown in FIGS. 1A-1C, the blend is then contacted with a patterned substrate, i.e., a “master,” and heated to cure the PFPE precursor into a network.

In some embodiments, the PFPE precursor is fully cured to form a fully cured PFPE precursor. In some embodiments, the free radical curing reaction is allowed to proceed only partially to form a partially-cured network.

### III.D. Adhering a Layer of a PFPE Material to a Substrate Through a Thermal Free Radical Curing Process

In some embodiments the fully cured PFPE precursor is removed, e.g., peeled, from the patterned substrate, i.e., the master, after curing and contacted with a second substrate to form a reversible, hermetic seal.

In some embodiments, a partially cured network is contacted with a second partially cured layer of PFPE material and the curing reaction is taken to completion, thereby forming a permanent bond between the PFPE layers.

In some embodiments, a partial free-radical curing method is used to bond at least one layer of a partially-cured PFPE material to a substrate, thereby forming a device such as a medical device or a surgical device or the like. In some embodiments, the partial free-radical curing method is used to bond a plurality of layers of a partially-cured PFPE material to a substrate, thereby forming a device such as a medical device or a surgical device or the like. In some embodiments, the substrate is selected from a glass material, a quartz material, a silicon material, a fused silica material, a plastic material, combinations thereof, and the like. In some embodiments, the substrate is treated with a silane coupling agent.

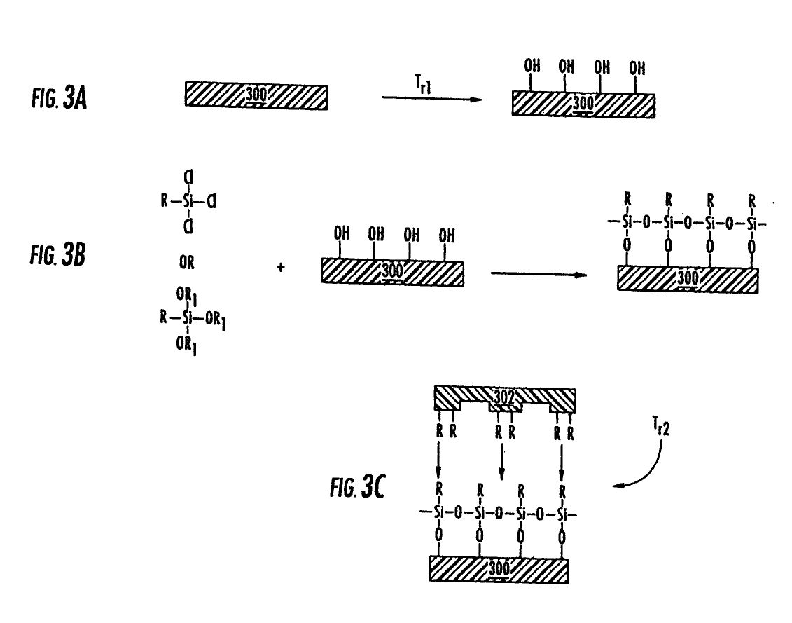

According to an embodiment, a layer of PFPE material can be adhered to a substrate as illustrated in FIGS. 3A-3C. Referring now to FIG. 3A, a substrate 300 is provided, wherein, in some embodiments, substrate 300 is selected from a glass material, a quartz material, a silicon material, a fused silica material, a plastic material, combinations thereof, and the like. Substrate 300 is then treated by treating process Tᵣ₁. In some embodiments, treating process Tᵣ₁ includes treating the substrate with a base/alcohol mixture, e.g., KOH/isopropanol, to impart a hydroxyl functionality to substrate 300.

Referring now to FIG. 3B, functionalized substrate 300 is reacted with a silane coupling agent, e.g., R—SiCl₃ or R—Si(OR₁)₃, wherein R and R₁ represent a functional group as described herein to form a silanized substrate 300. In some embodiments, the silane coupling agent is selected from a monohalosilane, a dihalosilane, a trihalosilane, a monoalkoxysilane, a dialkoxysilane, and a trialkoxysilane; and wherein the monohalosilane, dihalosilane, trihalosilane, monoalkoxysilane, dialkoxysilane, and trialkoxysilane are functionalized with a moieties selected from the group consisting of an amine, a methacrylate, an acrylate, a styrenic, an epoxy, an isocyanate, a halogen, an alcohol, a benzophenone derivative, a maleimide, a carboxylic acid, an ester, an acid chloride, and an olefin.

Referring now to FIG. 3C, silanized substrate 300 is contacted with a patterned layer of partially cured PFPE material 302 and treated by treating process Tr₂ to form a permanent bond between patterned layer of PFPE material 302 and substrate 300.

In some embodiments, a partial free radical cure is used to adhere a PFPE layer to a second polymeric material, such as a poly(dimethyl siloxane) (PDMS) material, a polyurethane material, a silicone-containing polyurethane material, and a PFPE-PDMS block copolymer material. In some embodiments, the second polymeric material includes a functionalized polymeric material. In some embodiments, the second polymeric material is encapped with a polymerizable group. In some embodiments, the polymerizable group is selected from an acrylate, a styrene, a methacrylate, combinations thereof, and the like. Further, in some embodiments, the second polymeric material can be treated with a plasma and a silane coupling agent to introduce the desired functionality to the second polymeric material.

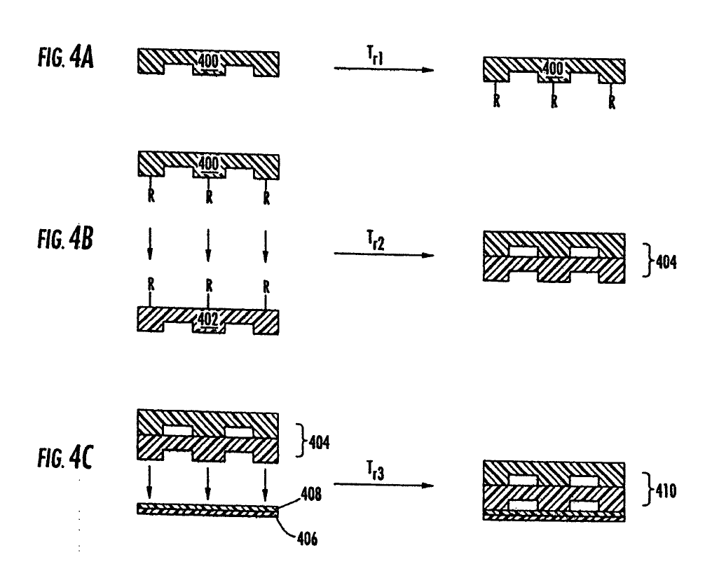

According to another embodiment, a patterned layer of PFPE material can be adhered to another patterned layer of polymeric material as illustrated in FIGS. 4A-4C. Referring now to FIG. 4A, a patterned layer of a first polymeric material 400 is provided. In some embodiments, first polymeric material includes a PFPE material. In some embodiments, first polymeric material includes a polymeric material selected from a poly(dimethylsiloxane) material, a polyurethane material, a silicone-containing polyurethane material, a PFPE-PDMS block copolymer material, combinations thereof, and the like. Patterned layer of first polymeric material 400 is then treated by treating process Tᵣ₁. In some embodiments, treating process Tᵣ₁ includes exposing the patterned layer of first polymeric material 400 to UV light in the presence of O₃ and an R functional group, to add an R functional group to the patterned layer of polymeric material 400.

Referring now to FIG. 4B, the functionalized patterned layer of first polymeric material 400 is contacted with the top surface of a functionalized patterned layer of PFPE material 402 and then treated by treating process Tᵣ₂ to form a two layer hybrid assembly 404. Thus, functionalized patterned layer of first polymeric material 400 is thereby bonded to functionalized patterned layer of PFPE material 402.

Referring now to FIG. 4C, two-layer hybrid assembly 404, in some embodiments, is contacted with substrate 406 to form a multilayer hybrid structure 410. In some embodiments, substrate 406 is coated with a layer of liquid PFPE precursor material 408. Multilayer hybrid structure 410 is treated by treating process Tᵣ₃ to bond two-layer assembly 404 to substrate 406.

IV. A Device Fabricated from a Two-Component Curing Process

The present subject matter provides a device by which a polymer, such as functional perfluoropolyether (PFPE) precursors, are contacted with a patterned surface and then cured through the reaction of two components, such as epoxy/amine, epoxy/hydroxyl, carboxylic acid/amine, carboxylic acid/hydroxyl, ester/amine, ester/hydroxyl, amine/anhydride, acid halide/hydroxyl, acid halide/amine, amine/halide, hydroxyl/halide, hydroxyl/chlorosilane, azide/acetylene and other so-called “click chemistry” reactions, and metathesis reactions involving the use of Grubb's-type catalysts to form a fully-cured or a partially-cured device.

As used herein the term “click chemistry” refers to a term used in the art to describe the synthesis of compounds using any of a number of carbon-heteroatom bond forming reactions. “Click chemistry” reactions typically are relatively insensitive to oxygen and water, have high stereoselectivity and yield, and thermodynamic driving forces of about 20 kcal/mol or greater. Useful “click chemistry” reactions include cycloaddition reactions of unsaturated compounds, including 1,3-dipolar additions and Diels-Alder reactions; nucleophilic substitution reactions, especially those involving ring opening of small, strained rings like epoxides and aziridines; addition reactions to carbon-carbon multiple bonds; and reactions involving non-aldol carbonyl chemistry, such as the formation of ureas and amides.

Further, the term “metathesis reactions” refers to reactions in which two compounds react to form two new compounds with no change in oxidation numbers in the final products. For example, olefin metathesis involves the 2+2 cycloaddition of an olefin and a transition metal alkylidene complex to form a new olefin and a new alkylidene. In ring-opening metathesis polymerization (ROMP), the olefin is a strained cyclic olefin, and 2+2 cycloaddition to the transition metal catalyst involves opening of the strained ring. The growing polymer remains part of the transition metal complex until capped, for example, by 2+2 cycloaddition to an aldehyde. Grubbs catalysts for metathesis reactions were first described in 1996 (see Schwab, P., et al., J. Am. Chem. Soc., 118, 100-110 (1996)). Grubbs catalysts are transition metal alkylidenes containing ruthenium supported by phosphine ligands and are unique in that that they are tolerant of different functionalities in the alkene ligand.

Accordingly, in an embodiment, the photocurable component can include functional groups that can undergo photochemical 2+2 cycloadditions. Such groups include alkenes, aldehydes, ketones, and alkynes. Photochemical 2+2 cycloadditions can be used, for example, to form cyclobutanes and oxetanes.

Thus, in some embodiments, the partially-cured device can be contacted with another substrate, and the curing is then taken to completion to adhere the material of the device to the substrate. This method can be used to adhere multiple layers of polymer devices, such as for example PFPE material, to another substrate or device.

Further, in some embodiments, the substrate includes a second polymeric material, such as PDMS, or another polymer. In some embodiments, the second polymeric material includes an elastomer other than PDMS, such as Kratons™ (Shell Chemical Company), buna rubber, natural rubber, a fluoroelastomer, chloroprene, butyl rubber, nitrile rubber, polyurethane, or a thermoplastic elastomer. In some embodiments, the second polymeric material includes a rigid thermoplastic material, including but not limited to: polystyrene, poly(methyl methacrylate), a polyester, such as poly(ethylene terephthalate), a polycarbonate, a polyimide, a polyamide, a polyvinylchloride, a polyolefin, a poly(ketone), a poly(ether ether ketone), and a poly(ether sulfone).

In some embodiments, the PFPE layer is adhered to a solid substrate, such as a glass material, a quartz material, a silicon material, a fused silica material, combinations thereof, and the like through use of a silane coupling agent.

### IV.A. A Patterned PFPE Layer Formed Through a Two-Component Curing Process

In some embodiments, a polymeric device, such as a medical or surgical device is formed through the reaction of a two-component functional liquid precursor system. Using the general method for forming a patterned layer of polymeric material as described herein, a liquid precursor material that includes a two-component system is contacted with a patterned substrate and a patterned layer of polymeric material is formed. For discussion purposes throughout this specification polymeric material for fabricating the devices disclosed herein will be described with reference to PFPE materials, however, it will be appreciated that other polymers are suitable for such applications and an understanding of how to generally manipulate other such polymers for use in the present described example will be appreciated by one of ordinary skill in the art. In some embodiments, the two-component liquid precursor system is selected from an epoxy/amine, epoxy/hydroxyl, carboxylic acid/amine, carboxylic acid/hydroxyl, ester/amine, ester/hydroxyl, amine/anhydride, acid halide/hydroxyl, acid halide/amine, amine/halide, hydroxyl/halide, hydroxyl/chlorosilane, azide/acetylene and other so-called “click chemistry” reactions, and metathesis reactions involving the use of Grubb's-type catalysts. The functional liquid precursors are blended in the appropriate ratios and then contacted with a patterned surface or master. The curing reaction is allowed to take place by using heat, catalysts, and the like, until the device is formed.

In some embodiments, a fully cured PFPE precursor is formed. In some embodiments, the two-component reaction is allowed to proceed only partially, thereby forming a partially cured PFPE network.

### IV.B. Adhering a PFPE Layer to a Substrate Through a Two-Component Curing Process

### IV.B.1. Full Cure with a Two-Component Curing Process

In some embodiments, the fully cured PFPE two-component precursor is removed, e.g., peeled, from the master following curing and contacted with a substrate to form a reversible, hermetic seal. In some embodiments, the partially cured network is contacted with another partially cured layer of PFPE and the reaction is taken to completion, thereby forming a permanent bond between the abutting layers.

### IV.B.2. Partial Cure with a Two-Component System

As shown in FIGS. 3A-3C, in some embodiments, the partial two-component curing technique is used to bond at least one layer of a partially-cured PFPE material to a substrate, thereby forming a component of a medical or surgical device. In some embodiments, the partial two-component curing is used to bond a plurality of layers of a partially-cured PFPE material to a substrate to form such devices. In some embodiments, the substrate is selected from a glass material, a quartz material, a silicon material, a fused silica material, a plastic material, combinations thereof, and the like. In some embodiments, the substrate is treated with a silane coupling agent.

As shown in FIGS. 4A-4C, in some embodiments, a partial two-component cure is used to adhere the PFPE layer to a second polymeric material, such as a poly(dimethylsiloxane) (PDMS) material. In some embodiments, the PDMS material includes a functionalized PDMS material. In some embodiments, the PDMS is treated with a plasma and a silane coupling agent to introduce the desired functionality to the PDMS material. In some embodiments, the PDMS material is encapped with a polymerizable group. In some embodiments, the polymerizable group includes an epoxide. In some embodiments, the polymerizable group includes an amine.

In some embodiments, the second polymeric material includes an elastomer other than PDMS, such as Kratons™, buna rubber, natural rubber, a fluoroelastomer, chloroprene, butyl rubber, nitrile rubber, polyurethane, or a thermoplastic elastomer. In some embodiments, the second polymeric material includes a rigid thermoplastic, including but not limited to: polystyrene, poly(methyl methacrylate), a polyester, such as poly(ethylene terephthalate), a polycarbonate, a polyimide, a polyamide, a polyvinylchloride, a polyolefin, a poly(ketone), a poly(ether ether ketone), and a poly(ether sulfone).

### IV.B.3. Excess Cure with a Two-Component System

A medical or surgical device can be formed from contacting a functional perfluoropolyether (PFPE) precursor with a patterned substrate and cured through the reaction of two components, such as epoxy/amine, epoxy/hydroxyl, carboxylic acid/amine, carboxylic acid/hydroxyl, ester/amine, ester/hydroxyl, amine/anhydride, acid halide/hydroxyl, acid halide/amine, amine/halide, hydroxyl/halide, hydroxyl/chlorosilane, azide/acetylene and other so-called “click chemistry” reactions, and metathesis reactions involving the use of Grubb's-type catalysts, to form a layer of cured PFPE material. In this particular method, the layer of cured PFPE material can be adhered to a second substrate by fully curing the layer with an excess of one component and contacting the layer of cured PFPE material with a second substrate having an excess of a second component in such a way that the excess groups react to adhere the layers.

Thus, in some embodiments, a two-component system, such as an epoxy/amine, epoxy/hydroxyl, carboxylic acid/amine, carboxylic acid/hydroxyl, ester/amine, ester/hydroxyl, amine/anhydride, acid halide/hydroxyl, acid halide/amine, amine/halide, hydroxyl/halide, hydroxyvchlorosilane, azide/acetylene and other so-called “click chemistry” reactions, and metathesis reactions involving the use of Grubb's-type catalysts, is blended. In some embodiments, at least one component of the two-component system is in excess of the other component. The reaction is then taken to completion by heating, using a catalyst, and the like, with the remaining cured network having a plurality of functional groups generated by the presence of the excess component.

In some embodiments, two layers of fully cured PFPE materials including complimentary excess groups are contacted with one another, wherein the excess groups are allowed to react, thereby forming a permanent bond between the layers of the device.

As shown in FIGS. 3A-3C, in some embodiments, a fully cured PFPE network including excess functional groups is contacted with a substrate. In some embodiments, the substrate is selected from the group consisting of a glass material, a quartz material, a silicon material, a fused silica material, a plastic material, combinations thereof, and the like. In some embodiments, the substrate is treated with a silane coupling agent such that the functionality on the coupling agent is complimentary to the excess functionality on the fully cured network. Thus, a permanent bond is formed to the substrate.

As shown in FIGS. 4A-4C, in some embodiments, the two-component excess cure is used to bond a PFPE network to a second polymeric material, such as a poly(dimethylsiloxane) PDMS material. In some embodiments, the PDMS material includes a functionalized PDMS material. In some embodiments, the PDMS material is treated with a plasma and a silane coupling agent to introduce the desired functionality. In some embodiments, the PDMS material is encapped with a polymerizable group. In some embodiments, the polymerizable material includes an epoxide. In some embodiments, the polymerizable material includes an amine.

In some embodiments, the second polymeric material includes an elastomer other than PDMS, such as Kratons™, buna rubber, natural rubber, a fluoroelastomer, chloroprene, butyl rubber, nitrile rubber, polyurethane, or a thermoplastic elastomer. In some embodiments, the second polymeric material includes a rigid thermoplastic, including but not limited to: polystyrene, poly(methyl methacrylate), a polyester, such as poly(ethylene terephthalate), a polycarbonate, a polyimide, a polyamide, a polyvinylchloride, a polyolefin, a poly(ketone), a poly(ether ether ketone), and a poly(ether sulfone).

### IV.B.4 Blending a Thermalcurable Component with a Photocurable Material

According to yet another embodiment, devices are formed from adhering multiple layers of materials together. In one embodiment, a two-component thermally curable material is blended with a photocurable material, thereby creating a multiple stage curing material. In certain embodiments, the two-component system can include functional groups, such as epoxy/amine, epoxy/hydroxyl, carboxylic acid/amine, carboxylic acid/hydroxyl, ester/amine, ester/hydroxyl, amine/anhydride, acid halide/hydroxyl, acid halide/amine, amine/halide, hydroxyl/halide, hydroxyl/chlorosilane, azide/acetylene and other so-called “click chemistry” reactions, and metathesis reactions involving the use of Grubb's-type catalysts. In one embodiment, the photocurable component can include such functional groups as: acrylates, styrenics, epoxides, cyclobutanes and other 2+2 cycloadditions.

In some embodiments, a two-component thermally curable material is blended in varying ratios with a photocurable material. In one embodiment, the material can then be deposited on a patterned substrate as described above. Such a system can be exposed to actinic radiation, e.g., UV light, and solidified into a network, while the thermally curable components are mechanically entangled in the network but remain unreacted. Layers of the material can then be prepared, for example, cut, trimmed, punched with inlet/outlet holes, and aligned in predetermined positions on a second, photocured layer. Once the photocured layers are aligned and sealed, the device can be heated to activate the thermally curable component within the layers. When the thermally curable components are activated by the heat, the layers are adhered together by reaction at the interface.

In some embodiments, the thermal reaction is taken to completion. In other embodiments, the thermal reaction is only done partially and multiple layers are adhered this way by repeating this process. In other embodiments, a multilayered device is formed and adhered to a final, non-patterned layer through the thermal cure.

In some embodiments, the thermal cure reaction is done first. The layer is then prepared, for example, cut, trimmed, punched with inlet/outlet holes, and aligned. Next, the photocurable component is activated by exposure to actinic radiation, e.g., UV light, and the layers are adhered by functional groups reacting at the interface between the layers.

In some embodiments, blended two-component thermally curable and photocurable materials are used to bond a PFPE network to a second polymeric material, such as a poly(dimethylsiloxane) PDMS material. In some embodiments, the PDMS material includes a functionalized PDMS material. As will be appreciated by one of ordinary skill in the art, the functionalized PDMS material is PDMS material that contains a reactive chemical group, as described elsewhere herein. In some embodiments, the PDMS material is treated with a plasma and a silane coupling agent to introduce the desired functionality. In some embodiments, the PDMS material is encapped with a polymerizable group. In some embodiments, the polymerizable material includes an epoxide. In some embodiments, the polymerizable material includes an amine.

In some embodiments, the second polymeric material includes an elastomer other than PDMS, such as Kratons™, buna rubber, natural rubber, a fluoroelastomer, chloroprene, butyl rubber, nitrile rubber, polyurethane, or a thermoplastic elastomer. In some embodiments, the second polymeric material includes a rigid thermoplastic, including but not limited to: polystyrene, poly(methyl methacrylate), a polyester, such as poly(ethylene terephthalate), a polycarbonate, a polyimide, a polyamide, a polyvinylchloride, a polyolefin, a poly(ketone), a poly(ether ether ketone), a poly(ether sulfone), combinations thereof, and the like.

In some embodiments, a blend of a photocurable PFPE liquid precursor and a two-component thermally curable PFPE liquid precursor is made in such a way that one component of the two component thermally curable blend is in excess of the other. In this way, multiple layers can be adhered through residual complimentary functional groups present in multiple layers.

According to a preferred embodiment, the amount of thermal cure and photocure substance added to the material is selected to produce adhesion between layers of the completed device that can withstand a predetermined pressure, tension, torsion, compression, or the like without delaminating.

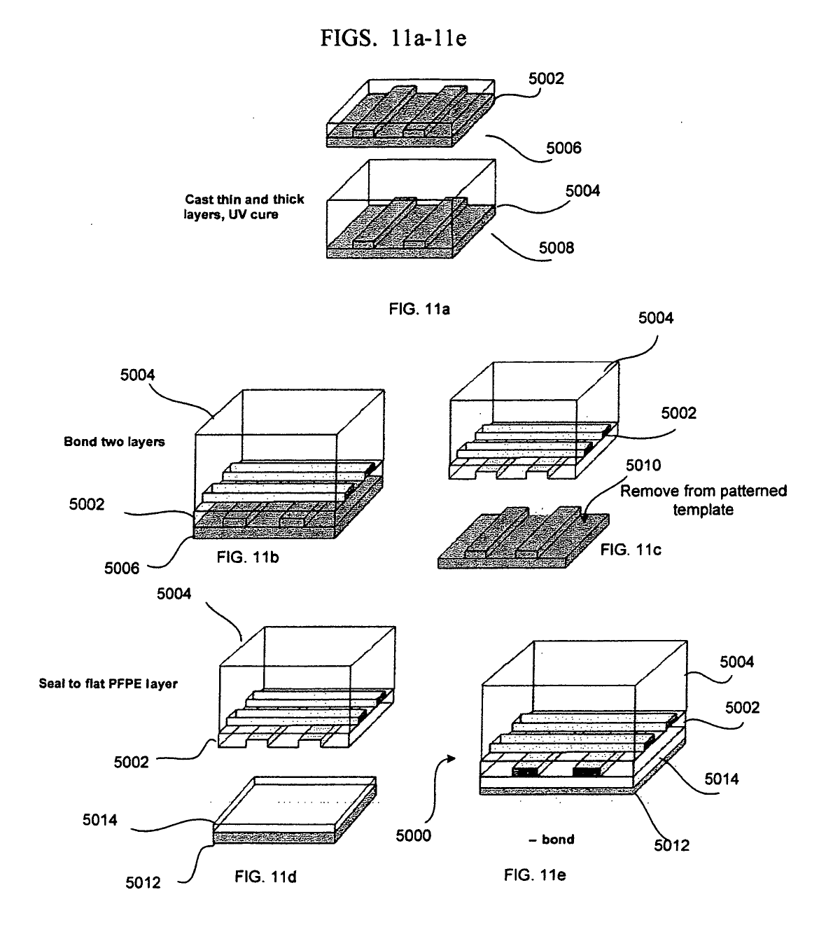

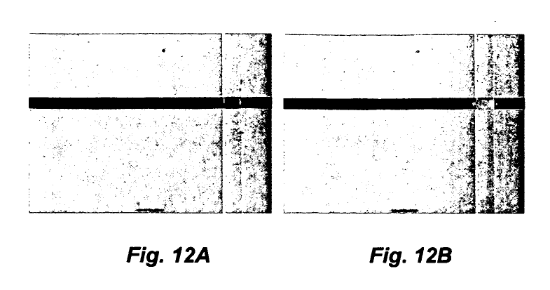

An illustrative example of a method for making a multilayered device will now be described with respect to FIGS. 11a-11e. A two-component thermally curable material blended with a photocurable material is disposed on patterned templates 5006, 5008 (sometimes referred to as a master template or template), as shown in FIG. 11a. According to alternative embodiments of the present invention, the blended material can be spin coated onto the patterned template or cast onto the patterned template by pooling the material inside a gasket. Typically, spin coating is used to form thin layers such as first layer 5002 and a cast technique is used to form thick layers such as second layer 5004, as will be appreciated by one of ordinary skill in the art. Next, the blended material positioned on templates 5006 and 5008 is treated with an initial cure, such as a photocure, to form first layer 5002 and second layer 5004, respectively. The photocure partially cures the material but does not initiate the thermal cure components of the material. Patterned template 5008 is then removed from second layer 5004. Removal of patterned templates from the layers is described in more detail herein. Next, second layer 5004 is positioned with respect to first layer 5002 and the combination is treated with a second cure, as shown in FIG. 11b, which results in the bonding, or adhesion, between first layer 5002 and second layer 5004, collectively referred to hereinafter as the “two adhered layers 5002 and 5004.” Typically, the second cure is an initial heat curing that initiates the two-component thermal cure of the material. Next, the two adhered layers 5002 and 5004 are removed from patterned template 5006, as shown in FIG. 11c. In FIG. 11d, the two adhered layers 5002 and 5004 are positioned on flat layer 5014, flat layer 5014 previously being coated onto flat template 5012 and treated with an initial cure. The combination of layers 5002, 5004, and 5014 is then treated to a final cure to fully adhere all three layers together, as shown in FIG. 11e.

According to alternative embodiments, patterned template 5006 can be coated with release layer 5010 to facilitate removal of the cured or partially cured layers (see FIG. 11c). Further, coating of the templates, e.g., patterned template 5006 and/or patterned template 5008, can reduce reaction of the thermal components with latent groups present on the template. For example, release layer 5010 can be a Gold/Palladium coating.

According to alternative embodiments, removal of the partially cured and cured layers can be realized by peeling, suction, pneumatic pressure, through the application of solvents to the partially cured or cured layers, or through a combination of these teachings.

V. Functionalizing a Surface of a Device

In some embodiments, a surface of a device can be functionalized to yield predetermined properties. In some embodiments, such functionalization includes, but is not limited to, the synthesis and/or attachment of peptides and other natural polymers to the surface of a device. Accordingly, the presently disclosed subject matter can be applied to devices, such as those described by Rolland, J. et al., JACS 2004, 126, 2322-2323, the disclosure of which is incorporated herein by reference in its entirety.

In some embodiments, the method includes binding a small molecule to the surface of a device. In such embodiments, once bound, the small molecule can serve a variety of functions. In some embodiments, the small molecule functions as a cleavable group, which when activated, can change the polarity of the surface and hence the wettability of the surface. In some embodiments, the small molecule functions as a binding site. In some embodiments, the small molecule functions as a binding site for one of a catalyst, a drug, a substrate for a drug, an analyte, and a sensor. In some embodiments, the small molecule functions as a reactive functional group. In some embodiments, the reactive functional group is reacted to yield a Zwitterion. In some embodiments, the Zwitterion provides a polar, ionic channel.

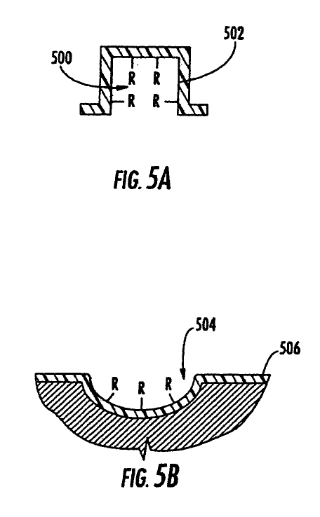

An embodiment of the presently disclosed method for functionalizing the surface of a device is illustrated in FIGS. 5A and 5B. Referring now to FIG. 5A, a device 500 is provided. In some embodiments, device 500 is formed from a functional PFPE material having an R functional group, as described herein. In some embodiments, device 500 includes a PFPE network which undergoes a post-curing treating process, whereby functional group R is introduced into the surface 502 of device 500.

Referring now to FIG. 5B, device 504 is provided. In some embodiments, device 504 is coated with a layer of functionalized PFPE material 506, which includes an R functional group, to impart functionality into device 504.

### V.A. Attaching a Functional Grout to a PFPE Network

In some embodiments, PFPE networks including excess functionality are used to functionalize the surface of a medical or surgical device. In some embodiments, the surface of a device is functionalized by attaching a functional moiety selected from a protein, an oligonucleotide, a drug, a ligand, a catalyst, a dye, a sensor, an analyte, and a charged species capable of changing the wettability of the device.

In some embodiments, latent functionalities are introduced into the fully cured PFPE network. In some embodiments, latent methacrylate groups are present at the surface of the PFPE network that has been free radically cured either photochemically or thermally. Multiple layers of fully cured PFPE are then contacted with the functionalized surface of the PFPE network, forming a seal, and reacted, by heat, for example, to allow the latent functionalities to react and form a permanent bond between the layers.

In some embodiments, the latent functional groups react photochemically with one another at a wavelength different from that used to cured PFPE precursors. In some embodiments, this method is used to adhere fully cured layers to a substrate. In some embodiments, the substrate is selected from the group consisting of a glass material, a quartz material, a silicon material, a fused silica material, and a plastic material. In some embodiments, the substrate is treated with a silane coupling agent complimentary to the latent functional groups.

In some embodiments, such latent functionalities are used to adhere a fully cured PFPE network to a second polymeric material, such as a poly(dimethylsiloxane) PDMS material. In some embodiments, the PDMS material includes a functionalized PDMS material. In some embodiments, the PDMS material is treated with a plasma and a silane coupling agent to introduce the desired functionality. In some embodiments, the PDMS material is encapped with a polymerizable group. In some embodiments, the polymerizable group is selected from the group consisting of an acrylate, a styrene, and a methacrylate.

In some embodiments, the second polymeric material includes an elastomer other than PDMS, such as Kratons™, buna rubber, natural rubber, a fluoroelastomer, chloroprene, butyl rubber, nitrile rubber, polyurethane, or a thermoplastic elastomer. In some embodiments, the second polymeric material includes a rigid thermoplastic, including but not limited to: polystyrene, poly(methyl methacrylate), a polyester, such as poly(ethylene terephthalate), a polycarbonate, a polyimide, a polyamide, a polyvinylchloride, a polyolefin, a poly(ketone), a poly(ether ether ketone), and a poly(ether sulfone).

### V.B. Introducing Functionality in the Generation of a Liquid PFPE Precursor

The presently disclosed subject matter provides a method of forming a device by which a photochemically cured PFPE layer is placed in conformal contact with a second substrate thereby forming a seal. The PFPE layer is then heated at elevated temperatures to adhere the layer to the substrate through latent functional groups. In some embodiments, the second substrate also includes a cured PFPE layer. In some embodiments, the second substrate includes a second polymeric material, such as a poly(dimethylsiloxane) (PDMS) material.

In some embodiments, the second polymeric material includes an elastomer other than PDMS, such as Kratons™, buna rubber, natural rubber, a fluoroelastomer, chloroprene, butyl rubber, nitrile rubber, polyurethane, or a thermoplastic elastomer. In some embodiments, the second polymeric material includes a rigid thermoplastic, including but not limited to: polystyrene, poly(methyl methacrylate), a polyester, such as poly(ethylene terephthalate), a polycarbonate, a polyimide, a polyamide, a polyvinylchloride, a polyolefin, a poly(ketone), a poly(ether ether ketone), and a poly(ether sulfone).

In some embodiments, the latent groups include methacrylate units that are not reacted during the photocuring process. Further, in some embodiments, the latent groups are introduced in the generation of the liquid PFPE precursor. For example, in some embodiments, methacrylate units are added to a PFPE diol through the use of glycidyl methacrylate, the reaction of the hydroxy and the epoxy group generates a secondary alcohol, which can be used as a handle to introduce chemical functionality. In some embodiments, multiple layers of fully cured PFPE are adhered to one another through these latent functional groups. In some embodiments, the latent functionalities are used to adhere a fully cured PFPE layer to a substrate. In some embodiments, the substrate is selected from the group consisting of a glass material, a quartz material, a silicon material, a fused silica material, and a plastic material. In some embodiments, the substrate is treated with a silane coupling agent.

Further, this method can be used to adhere a fully cured PFPE layer to a second polymeric material, such as a poly(dimethylsiloxane) (PDMS) material. In some embodiments, the PDMS material includes a functionalized PDMS material. In some embodiments, the PDMS material is treated with a plasma and a silane coupling agent to introduce the desired functionality. In some embodiments, the PDMS material is encapped with a polymerizable group. In some embodiments, the polymerizable material is selected from the group consisting of an acrylate, a styrene, and a methacrylate.

In some embodiments, the second polymeric material includes an elastomer other than PDMS, such as Kratons™, buna rubber, natural rubber, a fluoroelastomer, chloroprene, butyl rubber, nitrile rubber, polyurethane, or a thermoplastic elastomer. In some embodiments, the second polymeric material includes a rigid thermoplastic, including but not limited to: polystyrene, poly(methyl methacrylate), a polyester, such as poly(ethylene terephthalate), a polycarbonate, a polyimide, a polyamide, a polyvinylchloride, a polyolefin, a poly(ketone), a poly(ether ether ketone), and a poly(ether sulfone).

In some embodiments, PFPE networks containing latent functionality are used to functionalize the surface of device. Examples include the attachment of proteins, oligonucleotides, drugs, ligands, catalysts, dyes, sensors, analytes, and charged species capable of changing the wettability of the device.

### V.C. Linking Multiple Chains of a PFPE Material with a Functional Linker Group

In some embodiments, functionality is added to a device by adding a chemical “linker” moiety to the elastomer itself. In some embodiments, a functional group is added along the backbone of the precursor material. An example of this method is illustrated in Scheme 6.

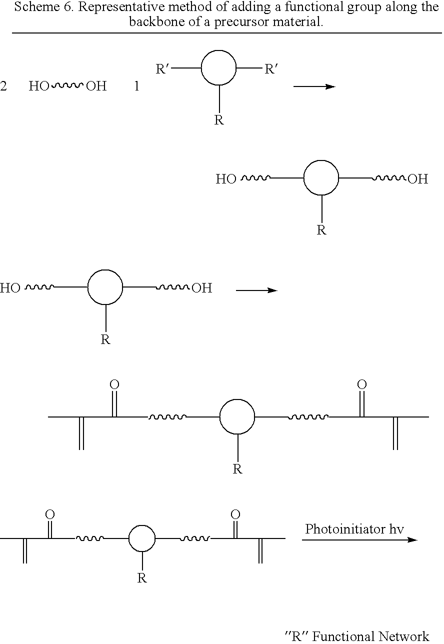

In some embodiments, the precursor material includes a macromolecule containing hydroxyl functional groups. In some embodiments, as depicted in Scheme 6, the hydroxyl functional groups include diol functional groups. In some embodiments, two or more of the diol functional groups are connected through a trifunctional “linker” molecule. In some embodiments, the trifunctional linker molecule has two functional groups, R and R′. In some embodiments, the R′ group reacts with the hydroxyl groups of the macromolecule. In Scheme 6, the circle can represent a linking molecule; and the wavy line can represent a PFPE chain.

In some embodiments, the R group provides the desired functionality to the surface of the device. In some embodiments, the R′ group is selected from the group including, but not limited to, an acid chloride, an isocyanate, a halogen, and an ester moiety. In some embodiments, the R group is selected from one of, but not limited to, a protected amine and a protected alcohol. In some embodiments, the macromolecule diol is functionalized with polymerizable methacrylate groups. In some embodiments, the functionalized macromolecule diol is cured and/or molded by a photochemical process as described by Rolland, J. et al. JACS 2004, 126, 2322-2323, the disclosure of which is incorporated herein by reference in its entirety.

Thus, the presently disclosed subject matter provides a method of incorporating latent functional groups into a photocurable PFPE material through a functional linker group. Thus, in some embodiments, multiple chains of a PFPE material are linked together before encapping the chain with a polymerizable group. In some embodiments, the polymerizable group is selected from a methacrylate, an acrylate, and a styrenic. In some embodiments, latent functionalities are attached chemically to such “linker” molecules in such a way that they will be present in the fully cured network.

In some embodiments, latent functionalities introduced in this manner are used to bond multiple layers of PFPE, bond a fully cured PFPE layer to a substrate, such as a glass material or a silicon material that has been treated with a silane coupling agent, or bond a fully cured PFPE layer to a second polymeric material, such as a PDMS material. In some embodiments, the PDMS material is treated with a plasma and a silane coupling agent to introduce the desired functionality. In some embodiments, the PDMS material is encapped with a polymerizable group. In some embodiments, the polymerizable group is selected from an acrylate, a styrene, and a methacrylate.

In some embodiments, the second polymeric material includes an elastomer other than PDMS, such as Kratons™, buna rubber, natural rubber, a fluoroelastomer, chloroprene, butyl rubber, nitrile rubber, polyurethane, or a thermoplastic elastomer. In some embodiments, the second polymeric material includes a rigid thermoplastic, including but not limited to: polystyrene, poly(methyl methacrylate), a polyester, such as poly(ethylene terephthalate), a polycarbonate, a polyimide, a polyamide, a polyvinylchloride, a polyolefin, a poly(ketone), a poly(ether ether ketone), and a poly(ether sulfone).

In some embodiments, PFPE networks including functionality attached to “linker” molecules are used to functionalize the surface of a device. In some embodiments, the device is functionalized by attaching a functional moiety selected from the group a protein, an oligonucleotide, a drug, a catalyst, a dye, a sensor, an analyte, and a charged species capable of changing the wettability of the device.

VI. Adding Functional Monomers to the PFPE Precursor Material

In some embodiments, a functional monomer can be added to an uncured precursor material. In some embodiments, the functional monomer is selected from functional styrenes, methacrylates, and acrylates. In some embodiments, the precursor material includes a fluoropolymer. In some embodiments, the functional monomer includes a highly fluorinated monomer. In some embodiments, the highly fluorinated monomer includes perfluoro ethyl vinyl ether (EVE). In some embodiments, the precursor material includes a poly(dimethyl siloxane) (PDMS) elastomer. In some embodiments, the precursor material includes a polyurethane elastomer. In some embodiments, the method further includes incorporating the functional monomer into the network by a curing step.

In some embodiments, functional monomers are added directly to the liquid PFPE precursor to be incorporated into the network upon crosslinking. For example, monomers can be introduced into the network that are capable of reacting post-crosslinking to adhere multiple layers of PFPE, bond a fully cured PFPE layer to a substrate, such as a glass material or a silicon material that has been treated with a silane coupling agent, or bond a fully cured PFPE layer to a second polymeric material, such as a PDMS material. In some embodiments, the PDMS material is treated with a plasma and a silane coupling agent to introduce the desired functionality. In some embodiment, the PDMS material is encapped with a polymerizable group. In some embodiments, the polymerizable material is selected from an acrylate, a styrene, and a methacrylate.

In some embodiments, the second polymeric material includes an elastomer other than PDMS, such as Kratons™, buna rubber, natural rubber, a fluoroelastomer, chloroprene, butyl rubber, nitrile rubber, polyurethane, or a thermoplastic elastomer. In some embodiments, the second polymeric material includes a rigid thermoplastic, including but not limited to: polystyrene, poly(methyl methacrylate), a polyester, such as poly(ethylene terephthalate), a polycarbonate, a polyimide, a polyamide, a polyvinylchloride, a polyolefin, a poly(ketone), a poly(ether ether ketone), and a poly(ether sulfone).

In some embodiments, functional monomers are added directly to the liquid PFPE precursor and are used to attach a functional moiety selected from a protein, an oligonucleotide, a drug, a catalyst, a dye, a sensor, an analyte, and a charged species capable of changing the wettability of the device.

Such monomers include, but are not limited to, tert-butyl methacrylate, tert butyl acrylate, dimethylaminopropyl methacrylate, glycidyl methacrylate, hydroxy ethyl methacrylate, aminopropyl methacrylate, allyl acrylate, cyano acrylates, cyano methacrylates, trimethoxysilane acrylates, trimethoxysilane methacrylates, isocyanato methacrylate, lactone-containing acrylates and methacrylates, sugar-containing acrylates and methacrylates, poly-ethylene glycol methacrylate, nornornane-containing methacrylates and acrylates, polyhedral oligomeric silsesquioxane methacrylate, 2-trimethylsiloxyethyl methacrylate, 1H,1H,2H,2H-fluoroctylmethacrylate, pentafluorostyrene, vinyl pyridine, bromostyrene, chlorostyrene, styrene sulfonic acid, fluorostyrene, styrene acetate, acrylamide, and acrylonitrile.

In some embodiments, monomers which already have the above agents attached are blended directly with the liquid PFPE precursor to be incorporated into the network upon crosslinking. In some embodiments, the monomer includes a group selected from a polymerizable group, the desired agent, and a fluorinated segment to allow for miscibility with the PFPE liquid precursor. In some embodiments, the monomer does not include a polymerizable group, the desired agent, and a fluorinated segment to allow for miscibility with the PFPE liquid precursor.

In some embodiments, monomers are added to adjust the mechanical properties of the fully cured elastomer. Such monomers include, but are not limited to: perfluoro(2,2-dimethyl-1,3-dioxole), hydrogen-bonding monomers which contain hydroxyl, urethane, urea, or other such moieties, monomers containing bulky side group, such as tert-butyl methacrylate.

In some embodiments, functional species such as the above mentioned monomers are introduced and are mechanically entangled, i.e., not covalently bonded, into the network upon curing. For example, in some embodiments, functionalities are introduced to a PFPE chain that does not contain a polymerizable monomer and such a monomer is blended with the curable PFPE species. In some embodiments, such entangled species can be used to adhere multiple layers of cured PFPE together if two species are reactive, such as: epoxy/amine, hydroxy/acid chloride, hydroxy/isocyanate, amine/isocyanate, amine/halide, hydroxy/halide, amine/ester, and amine/carboxylic acid. Upon heating, the functional groups will react and adhere the two layers together.

Additionally, such entangled species can be used to adhere a PFPE layer to a layer of another material, such as glass, silicon, quartz, PDMS, Kratons™, buna rubber, natural rubber, a fluoroelastomer, chloroprene, butyl rubber, nitrile rubber, polyurethane, or a thermoplastic elastomer. In some embodiments, the second polymeric material includes a rigid thermoplastic, including but not limited to: polystyrene, poly(methyl methacrylate), a polyester, such as poly(ethylene terephthalate), a polycarbonate, a polyimide, a polyamide, a polyvinylchloride, a polyolefin, a poly(ketone), a poly(ether ether ketone), and a poly(ether sulfone).

VII. Introducing Functionality to a PFPE Surface

In some embodiments, an Argon plasma is used to introduce functionality along a fully cured PFPE surface using the method for functionalizing a poly(tetrafluoroethylene) surface as described by Chen. Y. and Momose, Y. Surf. Interface. Anal. 1999, 27, 1073-1083, which is incorporated herein by reference in it entirety. More particularly, without being bound to any one particular theory, exposure of a fully cured PFPE material to Argon plasma for a period of time adds functionality along the fluorinated backbone.

Such functionality can be used to adhere multiple layers of PFPE, bond a fully cured PFPE layer to a substrate, such as a glass material or a silicon material that has been treated with a silane coupling agent, or bond a fully cured PFPE layer to a second polymeric material, such as a PDMS material. In some embodiments, the PDMS material includes a functionalized material. In some embodiments, the PDMS material is treated with a plasma and a silane coupling agent to introduce the desired functionality. Such functionalities also can be used to attach proteins, oligonucleotides, drugs, catalysts, dyes, sensors, analytes, and charged species capable of changing the wettability of the device fabricated from the materials.

In some embodiments, the second polymeric material includes an elastomer other than PDMS, such as Kratons™, buna rubber, natural rubber, a fluoroelastomer, chloroprene, butyl rubber, nitrile rubber, polyurethane, or a thermoplastic elastomer. In some embodiments, the second polymeric material includes a rigid thermoplastic, including but not limited to: polystyrene, poly(methyl methacrylate), a polyester, such as poly(ethylene terephthalate), a polycarbonate, a polyimide, a polyamide, a polyvinylchloride, a polyolefin, a poly(ketone), a poly(ether ether ketone), and a poly(ether sulfone).

In some embodiments, a fully cured PFPE layer is brought into conformal contact with a solid substrate. In some embodiments, the solid substrate is selected from the group consisting of a glass material, a quartz material, a silicon material, a fused silica material, and a plastic material. In some embodiments, the PFPE material is irradiated with UV light, e.g., a 185-nm UV light, which can strip a fluorine atom off of the back bone and form a chemical bond to the substrate as described by Vurens, G., et al. Langmuir 1992, 8, 1165-1169. Thus, in some embodiments, the PFPE layer is covalently bonded to the solid substrate by radical coupling following abstraction of a fluorine atom.

VIII. Adhesion of a Device to a Substrate Through an Encasing Polymer

In some embodiments, a device can be adhered to a substrate by placing the fully cured device in conformal contact on the substrate and pouring an “encasing polymer” over the entire device. In some embodiments, the encasing polymer is selected from the group consisting of a liquid epoxy precursor and a polyurethane. The encasing polymer is then solidified by curing or other methods. The encasement serves to bind the layers together mechanically and to bind the layers to the substrate. In some embodiments, the device includes one of a perfluoropolyether material as described in Section II.A and Section II.B. hereinabove and a fluoroolefin-based material as described in Section II.C. hereinabove.

In some embodiments, the substrate is selected from a glass material, a quartz material, a silicon material, a fused silica material, a plastic material, combinations thereof, and the like. Further, in some embodiments, the substrate includes a second polymeric material, such as poly(dimethylsiloxane) (PDMS), or another polymer. In some embodiments, the second polymeric material includes an elastomer other than PDMS, such as Kratons™, buna rubber, natural rubber, a fluoroelastomer, chloroprene, butyl rubber, nitrile rubber, polyurethane, or a thermoplastic elastomer. In some embodiments, the second polymeric material includes a rigid thermoplastic material, including but not limited to: polystyrene, poly(methyl methacrylate), a polyester, such as poly(ethylene terephthalate), a polycarbonate, a polyimide, a polyamide, a polyvinylchloride, a polyolefin, a poly(ketone), a poly(ether ether ketone), and a poly(ether sulfone). In some embodiments, the surface of the substrate is functionalized with a silane coupling agent such that it will react with the encasing polymer to form an irreversible bond.

IX. Forming a Microstructure Using Sacrificial Layers

In some embodiments, microstructures of devices can be formed by utilizing sacrificial layers including a degradable or selectively soluble material during fabrication of the device. In some embodiments, the method includes contacting a liquid precursor material with a two-dimensional or a three-dimensional sacrificial structure, treating, e.g., curing, the precursor material, and removing the sacrificial structure to form a microstructure of the device.

Accordingly, in some embodiments, a PFPE liquid precursor is disposed on a multidimensional scaffold, wherein the multidimensional scaffold is fabricated from a material that can be degraded or washed away after curing of the PFPE network. These materials protect the microstructures from being filled in when another layer of elastomer is cast thereon. Examples of such degradable or selective soluble materials include, but are not limited to waxes, photoresists, polysulfones, polylactones, cellulose fibers, salts, or any solid organic or inorganic compounds. In some embodiments, the sacrificial layer is removed thermally, photochemically, or by washing with solvents. The PFPE materials of use in forming a microstructure by using sacrificial layers include those PFPE and fluoroolefin-based materials as described herein.

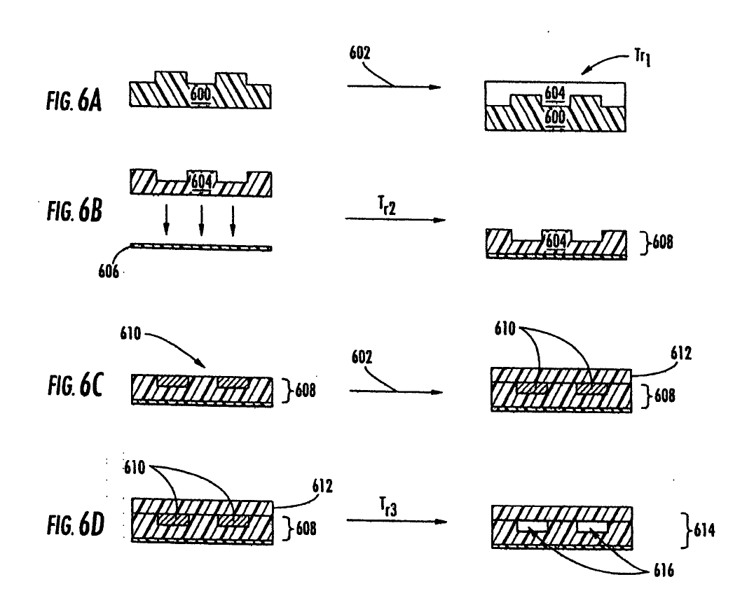

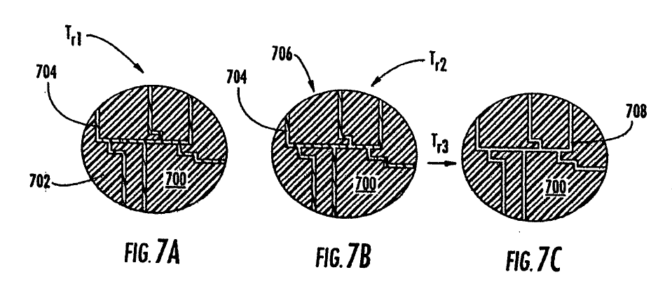

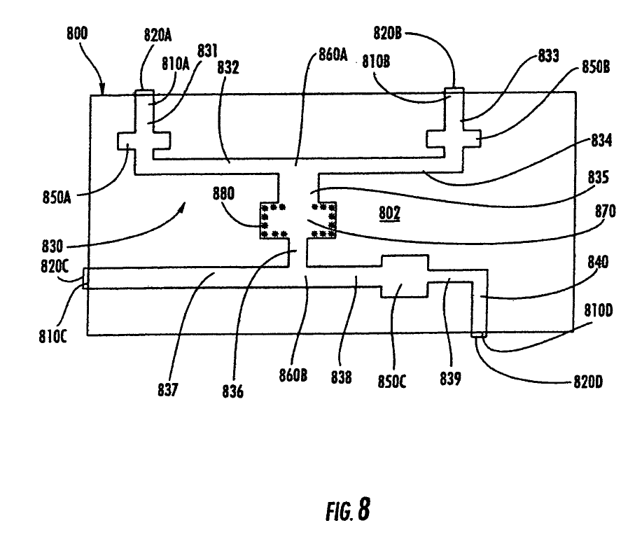

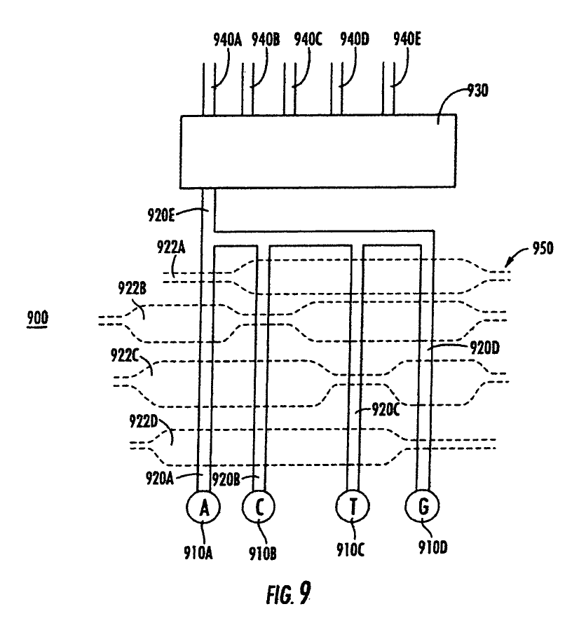

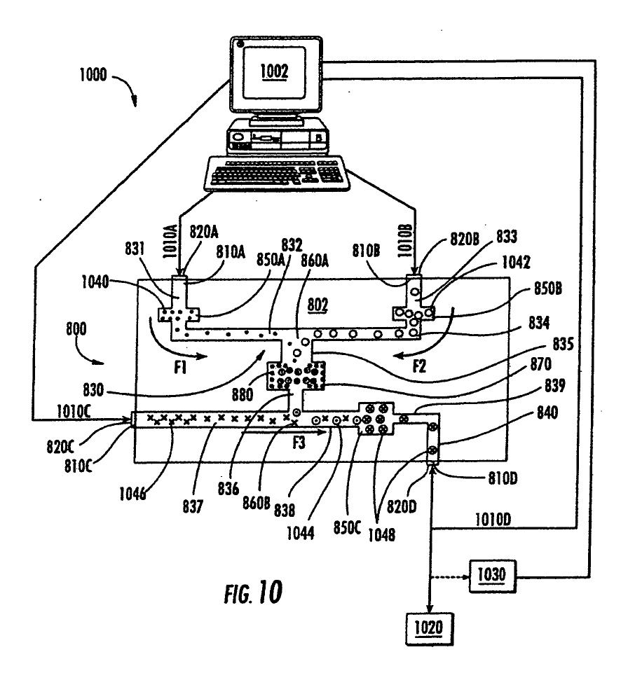

FIGS. 6A-6D and FIGS. 7A-7C show embodiments of forming a microstructure by using a sacrificial layer of a degradable or selectively soluble material. Referring now to FIG. 6A, a patterned substrate 600 is provided. Liquid PFPE precursor material 602 is disposed on patterned substrate 600. In some embodiments, liquid PFPE precursor material 602 is disposed on patterned substrate 600 via a spin-coating process. Liquid PFPE precursor material 602 is treated by treating process Tᵣ₁ to form a layer of treated liquid PFPE precursor material 604.

Referring now to FIG. 6B, the layer of treated liquid PFPE precursor material 604 is removed from patterned substrate 600. In some embodiments, the layer of treated liquid PFPE precursor material 604 is contacted with substrate 606. In some embodiments, substrate 606 includes a planar substrate or a substantially planar substrate. In some embodiments, the layer of treated liquid PFPE precursor material is treated by treating process Tᵣ₂, to form two-layer assembly 608.

Referring now to FIG. 6C, a predetermined volume of degradable or selectively soluble material 610 is disposed on two-layer assembly 608. In some embodiments, the predetermined volume of degradable or selectively soluble material 610 is disposed on two-layer assembly 608 via a spin-coating process. Referring once again to FIG. 6C, liquid precursor material 602 is disposed on two-layer assembly 608 and treated to form a layer of PFPE material 612, which covers the predetermined volume of degradable or selectively soluble material 610.

Referring now to FIG. 6D, the predetermined volume of degradable or selectively soluble material 610 is treated by treating process Tᵣ₃ to remove the predetermined volume of degradable or selectively soluble material 610, thereby forming microstructure 616. In some embodiments, treating process Tᵣ₃ is selected from the group of a thermal process, an irradiation process, and a dissolution process.

In some embodiments, patterned substrate 600 includes an etched silicon wafer. In some embodiments, the patterned substrate includes a photoresist patterned substrate. For the purposes of the presently disclosed subject matter, the patterned substrate can be fabricated by any of the processing methods known in the art, including, but not limited to, photolithography, electron beam lithography, and ion milling.

In some embodiments, degradable or selectively soluble material 610 is selected from the group of a polyolefin sulfone, a cellulose fiber, a polylactone, and a polyelectrolyte. In some embodiments, the degradable or selectively soluble material 610 is selected from a material that can be degraded or dissolved away. In some embodiments, degradable or selectively soluble material 610 is selected from the group of salt, water-soluble polymer, solvent-soluble polymer, combinations thereof, and the like.

FIGS. 7A-C illustrate forming a microstructure through the use of a sacrificial layer. Referring now to FIG. 7A, a substrate 700 is provided. In some embodiments, substrate 700 is coated with a liquid PFPE precursor material 702. Sacrificial structure 704 is placed on substrate 700. In some embodiments, liquid PFPE precursor material 702 is treated by treating process Tᵣ₁.

Referring now to FIG. 7B, a second liquid PFPE precursor material 706 is disposed over sacrificial structure 704, in such a way to encase sacrificial structure 704 in second liquid precursor material 706. Second liquid precursor material 706 is then treated by treating process Tᵣ₂. Referring now to FIG. 7C, sacrificial structure 704 is treated by treating process Tᵣ₃, to degrade and/or remove sacrificial structure, thereby forming microstructure 708.

In some embodiments, substrate 700 includes a silicon wafer. In some embodiments, sacrificial structure 704 includes a degradable or selectively soluble material. In some embodiments, sacrificial structure 704 is selected from the group of a polyolefin sulfone, a cellulose fiber, a polylactone, and a polyelectrolyte. In some embodiments, the sacrificial structure 704 is selected from a material that can be degraded or dissolved away. In some embodiments, sacrificial structure 704 is selected from the group of a salt, a water-soluble polymer, and a solvent-soluble polymer.

### IX.I. Increasing the Modulus of a Device Using PTFE Powder

In some embodiments, the modulus of a device fabricated from PFPE materials or any of the fluoropolymer materials described herein can be increased by blending polytetrafluoroethylene (PTFE) powder, also referred to herein as a “PTFE filler,” into the liquid precursor prior to curing. Because PTFE itself has a very high modulus, addition of PTFE in its powder form, when evenly dispersed throughout the low modulus materials of the present invention, will raise the overall modulus of the material. The PTFE filler also can contribute additional chemical stability and solvent resistance to the PFPE materials.

### IX.II. Use of the Material in Combination with a Device

According to an embodiment of the present invention, micro or nano scale devices or particles made from the methods and materials described herein can be formed for incorporation into or association with a medical or surgical device. For example, micro or nano scale valves or plugs can be formed from the materials and methods of the present invention that can effectively close off channels in a device. According to one embodiment, the valve or plug can be formed in a shape and/or size configuration to fit within a micro-chamber and remain in position or be configured to move in response to substances flowing in a particular direction or block particular channels from flow. According to another embodiment, a valve or plug can be formed in a micro-channel by introducing the materials of the present invention, in liquid form, into the micro-channel and curing the liquid material according to the methods disclosed in the present invention. Thereby, the valve or plug takes on the shape of the micro-channel forming a conformal fit.

X. Using a Functionalized Perfluoropolyether Network as a Gas Separation Membrane

A functionalized perfluoropolyether (PFPE) network fabricated from the present disclosure can function as a gas separation, permeable, or semi-permeable membrane, hereinafter “gas membrane”. In some embodiments, the functionalized PFPE network is used as a gas membrane to separate gases selected from the group of CO₂, methane, hydrogen, CO, CFCs, CFC alternatives, organics, nitrogen, methane, H₂S, amines, fluorocarbons, fluoroolefins, and O₂. In some embodiments, the functionalized PFPE network is used to separate gases in a water purification process. In some embodiments, the gas membrane can be used as an oxygen permeable, bacteria impermeable membrane for medical applications. In some embodiments, the gas membrane can be used as a oxygen exchange membrane for medical applications, such as but not limited to blood vessels or artificial lung tissue. In some embodiments, the gas membrane includes a stand-alone film. In some embodiments, the gas membrane includes a composite film.

In some embodiments, the gas membrane includes a co-monomer. In some embodiments, the co-monomer regulates the permeability properties of the gas membrane. Further, the mechanical strength and durability of such membranes can be finely tuned by adding composite fillers, such as silica particles and others, to the membrane. Accordingly, in some embodiments, the membrane further includes a composite filler. In some embodiments, the composite filler includes silica particles.

XI. Applications of Solvent Resistant Low Surface Energy Materials

According to alternative embodiments, the presently disclosed materials and methods can be combined with and/or substituted for, one or more of the following materials and applications.

According to one embodiment, the materials and methods of the present invention can be substituted for the silicone component in adhesive materials. In another embodiment, the materials and methods of the present invention can be combined with adhesive materials to provide stronger binding and alternative adhesion formats. An example of a material to which the present invention can be applied includes adhesives, such as a two part flowable adhesive that rapidly cures when heated to form a flexible and high tear elastomer. Adhesives such as this are suitable for bonding silicone coated fabrics to each other and to various substrates. An example of such an adhesive is, DOW CORNING® Q5-8401 ADHESIVE KIT (Dow Corning Corp., Midland, Mich., United States of America).

According to another embodiment, the materials and methods of the present invention can be substituted for the silicone component in color masterbatches. In another embodiment, the materials and methods of the present invention can be combined with the components of color masterbatches to provide stronger binding and alternative binding formats. Examples of a color masterbatch suitable for use with the present invention include, but are not limited to, a range of pigment masterbatches designed for use with liquid silicone rubbers (LSR's), for example, SILASTIC® LPX RED IRON OXIDE 5 (Dow Corning Corp., Midland, Mich., United States of America).

According to yet another embodiment, the materials and methods of the present invention can be substituted for liquid silicone rubber materials. In another embodiment, the materials and methods of the present invention can be combined with liquid silicone rubber materials to provide stronger binding and alternative binding techniques of the present invention to the liquid silicone rubber material. Examples of liquid silicone rubber suitable for use or substitution with the present invention include, but are not limited to, liquid silicone rubber coatings, such as a two part solventless liquid silicone rubber that is both hard and heat stable. Similar liquid silicone rubber coatings show particularly good adhesion to polyamide as well as glass and have a flexible low friction and non-blocking surface, such products are represented by, for example, DOW CORNING® 3625 A&B KIT. Other such liquid silicone rubber includes, for example, DOW CORNING® 3629 PART A; DOW CORNING® 3631 PART A&B (a two part, solvent free, heat-cured liquid silicone rubber); DOW CORNING® 3715 BASE (a two part solventless silicone top coat that cures to a very hard and very low friction surface that is anti-soiling and dirt repellent); DOW CORNING® 3730 A&B KIT (a two part solventless and colorless liquid silicone rubber with particularly good adhesion to polyamide as well as glass fabric); SILASTIC® 590 LSR PART A&B (a two part solventless liquid silicone rubber that has good thermal stability); SILASTIC® 9252/250P KIT PARTS A & B (a two part, solvent-free, heat cured liquid silicone rubber; general purpose coating material for glass and polyamide fabrics; three grades are commonly available including halogen free, low smoke toxicity, and food grade); SILASTIC® 9252/500P KIT PARTS A&B; SILASTIC® 9252/900P KIT PARTS A&B; SILASTIC® 9280/30 KIT PARTS A & B; SILASTIC® 9280/60E KIT PARTS A & B; SILASTIC® 9280/70E KIT PARTS A & B; SILASTIC® 9280/75E KIT PARTS A & B; SILASTIC® LSR 9151-200P PART A; SILASTIC® LSR 9451-1000P; RTV Elastomers (Dow Corning Corp., Midland, Mich., United States of America); DOW CORNING® 734 FLOWABLE SEALANT, CLEAR (a one part solventless silicone elastomer for general sealing and bonding applications, this silicone elastomer is a flowable liquid that is easy to use and cures on exposure to moisture in the air); DOW CORNING® Q3-3445 RED FLOWABLE ELASTOMER; (a red, flowable one part solventless silicone elastomer for high temperature release coatings, typically this product is used to coat fabric, release foodstuffs, and is stable up to 260° C.); and DOW CORNING® Q3-3559 SEMIFLOWABLE TEXTILE ELASTOMER (a semi-flowable one part solventless silicone elastomer).

According to yet another embodiment, the materials and methods of the present invention can be substituted for water based precured silicone elastomers. In another embodiment, the materials and methods of the present invention can be combined with water based silicone elastomers to provide the improved physical and chemical properties described herein to the materials. Examples of water based silicone elastomers suitable for use or substitution with the present invention include, but are not limited to, water based auxiliaries to which the present invention typically applies include DOW CORNING® 84 ADDITIVE (a water based precured silicone elastomer); DOW CORNING® 85 ADDITIVE (a water based precured silicone elastomer); DOW CORNING® ET-4327 EMULSION (methyl/phenyl functional silicone emulsion providing fiber lubrication, abrasion resistance, water repellency and flexibility to glass fabric, typically used as a glassfiber pre-treatment for PTFE coatings); and Dow Corning 7-9120 Dimethicone NF Fluid (a new grade of polydimethylsiloxane fluid introduced by Dow Corning for use in over-the-counter (OTC) topical and skin care products).

According to yet another embodiment, the materials and methods of the present invention can be substituted for other silicone based materials. In another embodiment, the materials and methods of the present invention can be combined with such other silicone based materials to impart improved physical and chemical properties to these other silicone based materials. Examples of other silicone based materials suitable for use or substitution with the present invention include, but are not limited to, for example, United Chemical Technologies RTV silicone (United Chemical Technologies, Inc., Bristol, Pa., United States of America) (flexible transparent elastomer suited for electrical/electronic potting and encapsulation); Sodium Methyl siliconate (this product renders siliceous surfaces water repellent and increases green strength and green storage life); Silicone Emulsion (useful as a non-toxic sprayable releasing agent and dries to clear silicone film); PDMS/a-Methylstyrene (useful where temporary silicone coating must be dissolved off substrate); GLASSCLAD® 6C (United Chemical Technologies, Inc., Bristol, Pa., United States of America) (a hydrophobic coating with glassware for fiberoptics, clinical analysis, electronics); GLASSCLAD® 18 (a hydrophobic coating for labware, porcelain ware, optical fibers, clinical analysis, and light bulbs); GLASSCLAD® HT (a protective hard thin film coating with >350° C. stability); GLASSCLAD® PSA (a high purity pressure sensitive adhesive which forms strong temporary bonds to glass, insulation components, metals and polymers); GLASSCLAD® SO (a protective hard coating for deposition of silicon dioxide on silicon); GLASSCLAD® EG (a flexible thermally stable resin, gives oxidative and mechanical barrier for resistors and circuit boards); GLASSCLAD® RC (methylsilicone with >250° C. stability, commonly used as coatings for electrical and circuit board components); GLASSCLAD® CR (silicone paint formulation curing to a flexible film, serviceable to 290° C.); GLASSCLAD® TF (a source of thick film (0.2-0.4 micron) coatings of silicon dioxide, converts to 36% silicon dioxide and is typically used for dielectric layers, abrasion resistant coatings, and translucent films); GLASSCLAD® FF (a moisture activated soft elastomer for biomedical equipment and optical devices); and UV SILICONE (UV curable silicone with refractive index (R.I.) matched to silica, cures in thin sections with conventional UV sources).

According to still further embodiments of the present invention, the materials and methods of the present invention can be substituted for and/or combined with further silicone containing materials. Some examples of further silicone containing materials include, but are not limited to, TUFSIL® (Specialty Silicone Products, Inc., Ballston Spa, New York, United States of America) (developed by Specialty Silicones primarily for the manufacture of components of respiratory masks, tubing, and other parts that come in contact with skin, or are used in health care and food processing industries); Baysilone Paint Additive TP 3738 (LANXESS Corp., Pittsburgh, Pa., United States of America) (a slip additive that is resistant to hydrolysis); Baysilone Paint Additive TP 3739 (compositions that reduce surface tension and improve substrate wetting, three acrylic thickeners for anionic, cationic, nonionic and amphoteric solutions, such as APK, APN and APA which are powdered polymethacrylates, and a liquid acrylic thickener); Tego Protect 5000 (Tego Chemie Service GmbH, Essen, Germany) (a modified polydimethylsiloxane resin typically for matte finishes, clear finishes and pigmented paint systems); Tego Protect 5001 (a silicone polyacrylate resin that contains a water repellent, typically used with clear varnish systems); Tego Protect 5002 (a silicone polyacrylate resin that can be repainted after mild surface preparation); Microsponge 5700 Dimethicone (a system based on the Microsponge dimethicone entrapment technology which is useful in the production of emulsion, powder, and stick products for facial treatments, foundations, lipsticks, moisturizers, and sun care products, dimethicone typically is packed into the empty spaces in a complex crosslinked matrix of polymethacrylate copolymer); 350 cST polydimethylsiloxane makes up 78% of the entrapped dimethicone component and 1000 cST polydimethylsiloxane constitutes the other 22%, the system typically facilitates the delivery of dimethicone's protective action to the skin); MB50 high molecular weight polydimethylsiloxane additive series (enables better processing with reduced surface friction and faster operating speeds, commonly available in formulations for PE, PS, PP, thermoplastic polyester elastomer, nylon 6 and 66, acetal and ABS, the silicone component is odorless and colorless and can be used for applications involving food contact, the product can be used as a substitute for silicone fluid and PTFE); Slytherm XLT (a new polydimethylsiloxane low temperature heat transfer fluid from Dow Corning, unlike traditional organic transfer fluids, it is non-toxic, odorless and does not react with other materials in the system, at high temperatures it has the additional advantage of being non-fouling and non-sludge forming); and 561® silicone transformer fluid (this material has a flash point of 300° C. and a fire point of 343° C., the single-component fluid is 100% PDMS, contains no additives, is naturally degradable in soils and sediments, and does not cause oxygen depletion in water).

XII. Applications to Medical Devices and Medical Implants

The dual curable component materials of the present invention can be used in various medical applications including, but not limited to, medical device or medical implants. According to an embodiment, material including blended photo curing and thermal curing components can be used to fabricate medical devices, device components, portions of devices, surgical devices, tools, implantable components, and the like. The blended material refers to the mixing of photocurable and thermally curable constituents within the polymer that is to form the device. The use of such a system allows for the formation of discrete objects by activating the first curing system and then adhering such discrete objects to other objects, surfaces, or materials by activating the second curing system. In some embodiments, the dual cure materials can be used to make a medical device or implant outside the body through a first cure of the material, then the second cure can be utilized to adhered the device to tissue after implantation into the body. In other embodiments, the dual cure materials can be used to make medical devices or implants in stages and then the components can be cured together to form an implant. The medical devices or implants made with the dual cure materials of the present invention can be, but are not limited to, orthopedic devices, cardiovascular devices, intraluminal devices, dermatological devices, oral devices, optical devices, auditory devices, tissue devices, organ devices, neurological devices, vascular devices, reproductive devices, combinations thereof, and the like.

### XII.A. Forming Devices or Implants and Attaching Same to Another Device or Implant

According to some embodiments, an object, such as a component of a medical device or an implant, can be fabricated by forming a liquid precursor in a mold, activating a curing mechanism, such as photo curing or thermal curing to solidify or partially solidify the precursor, and removing the solid object from the mold. The object can then be placed in contact with another component of a medical device or implant, a surface, a coating, or the like and a second curing mechanism is activated, such as thermal curing or photo curing, to adhere the two objects together. For example, the object can be, but is not limited to, an artificial joint component, an artificial bone component, an artificial tooth or tooth component, an artificial articular surface, an artificial lens, and the like.

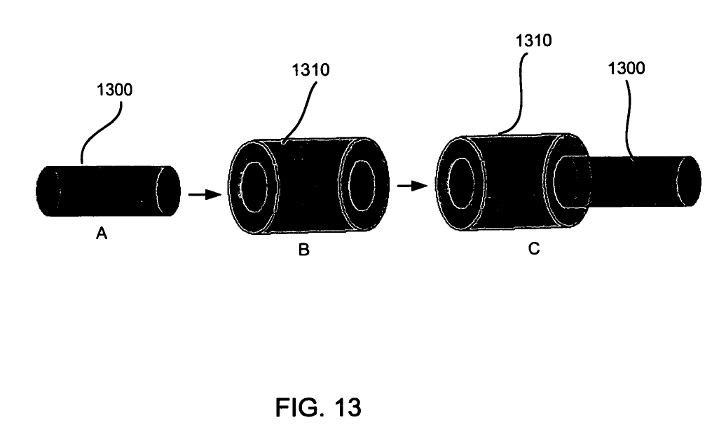

An example of this procedure is shown in FIG. 13, steps A-C, for example. According to FIG. 13, liquid PFPE material can be introduced into a mold and upon activating a first curing mechanism (e.g., thermal curing) the PFPE material is solidified to form a tube 1300. Tube 1300 can then be inserted into a second tube 1310, formed from the same or a different material, such as for example a different polymer or a natural material or structure such as tissue or a blood vessel. Next, a second curing mechanism (e.g., photo curing) is activated to adhere the PFPE tube 1300 to the second tube 1310.

According to embodiments directed to orthopedic applications, the dual curable materials of the present invention facilitate rebuilding and/or building new devices and structures for placement within a living body. Further embodiments include rebuilding and repairing existing biologic or artificial devices, tissues, and structures in situ. For example, dual curable materials may be utilized in building new joints and in repairing existing joints in vitro or in situ.

According to some embodiments, a damaged biologic component can be a damaged tissue such as skeletal tissue (e.g., spinal components such as discs and vertebral bodies, and other skeletal bones). In some embodiments, the dual cure materials of the present invention can be used in situ to augment the damaged biologic components. According to such embodiments, the method includes surgically inserting a mold structure into the damaged site or preparing the surgical site to act as a receiving mold for liquid dual cure material. The mold structure is configured to receive liquid dual curable material and is geometrically configured similar to the damaged biologic component to be replaced or configured to yield a desired result. Next, liquid dual cure material is introduced into the mold and first cured. The first cure can be, for example, treatment with light or heat. In some embodiments, the first cure can be an incomplete cure such that the replacement structure is left compliant. The compliant nature of the replacement structure can facilitate removal of the mold structure from the site of damage or positioning of the replacement component in the desired surgical/implant site. After removal of the mold structure, the first cured replacement structure can be treated with a second cure to further cure the replacement structure to satisfy desired mechanical properties for the particular application.

In other embodiments, the replacement component can be built up, either in vitro or in situ. According to these embodiments, an opening to a surgical site may be made smaller than an implant required by the site because the implant can be built up a portion at a time (e.g., replacing a hip joint through an arthroscopic type procedure). In such embodiments, dual cure liquid material, as described herein, can be introduced into a mold or a surgical site and treated with a first cure. The first cure activates the liquid material to form a first portion of the replacement component such that the component can retain a desired shape. The first portion can be configured to harden upon the first curing or remain compliant. Next, a second quantity of liquid material can be introduced to form a second portion of the replacement component. The material of the second portion is treated with a first cure treatment. The first cure treatment used to treat the second portion of the component can be the same technique used on the first portion such that each component retains a viable second cure component. Therefore, because each portion of the device retains a viable second cure component, after the first cured portions of the device are compiled to form the completed device, the portions can be treated with a second curing. Upon second curing, the second cure component of the layered portions of the device will be activated and the layered portion will bind together forming one integral device. Multiple portions of the replacement component can be formed, as described herein, as needed to make a replacement device. According to some embodiments, each portion can have different functional and/or mechanical properties to impart a desired mechanical and/or chemical result on the completed replacement component.

According to other embodiments, a portion such as an articular surface of a joint can be formed with the dual cure material of the present invention and attached to a natural joint in situ. According to such embodiments, an artificial articular surface can be fabricated from a first cure (e.g., thermal cure) of the dual cure material of the present invention. The artificial articular surface can then be implanted onto a preexisting joint surface and treated with a second cure (e.g., photo cure) such that the artificial articular surface binds to the preexisting joint surface.

### XII.B. Forming Devices or Implants and Attaching Same to Tissue

According to other embodiments, dual cure materials of the present invention can be used to replace or augment natural biologic tissue or structures and can be adhered directly to the tissue.

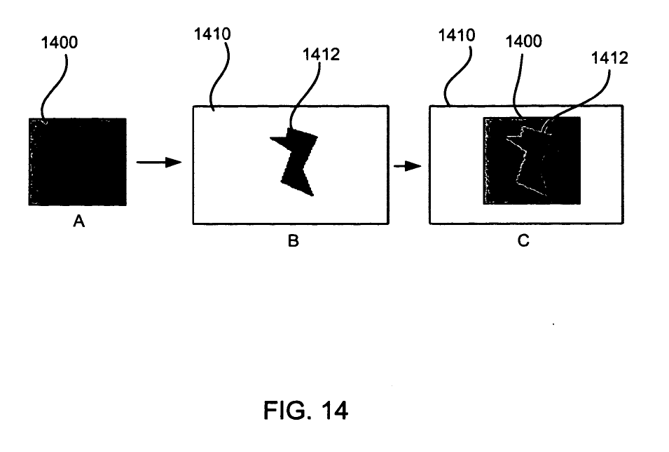

According to some embodiments, dual cure materials described herein may be incorporated into various types of patches, as shown in FIG. 14. In one embodiment, such a patch can be utilized in lung surgical procedures. Patches include, for example, but are not limited to sheets of dual cure material configured to be attached and secured directly to living tissue through activation of the second curing mechanism of the dual cure materials.

According to some embodiments, a disrupted or damaged material, device, or surface can be patched with material of the present invention. As shown in FIG. 14, steps A-C, a patch can be made by molding dual cure material into a desired shape and activating a first cure (e.g., thermal cure) to form patch 1400. Next, patch 1400 is placed over a device or tissue 1410 that is affected with a disruption or damage (e.g., a crack, hole, surgically altered tissue) 1412. After placement of patch 1400 over disruption 1412, a second curing mechanism is activated (e.g., photo curing) to adhere the patch to the surface of device 1410. The strength of the patch is dependent upon multiple variables, such as, for example, the size of binding area between patch 1400 and tissue 1410, the extent of curing administered to the patch/tissue combination, the chemicals, quantities, concentrations, and the like used in the second curing process, the composition of patch 1400, the composition of tissue or device 1410, combinations thereof, and the like. According to alternative embodiments, patch 1400 can undergo a second cure (e.g., thermal or photo curing) to attach patch 1400 to a compound, material, or substance that is known to bind to tissue. For example, patch 1400 can be treated with or adhered to a fribrin sealant component or glue, which is well known and used extensively in various clinical settings for adhering tissues together. In other embodiment, the patch can be second cure attached to a biocompatible material and the biocompatible material is then stitched to the tissue, thereby implanting the patch.

In yet other embodiments, the materials of the present invention can be used to fabricate a mold and replicate another object. In some embodiments the object to be molded and replicated can be a medical device or a tissue, such as a joint component, organ, organ scaffold, joint, skeletal component, dental component, ocular component, vascular component, and the like.

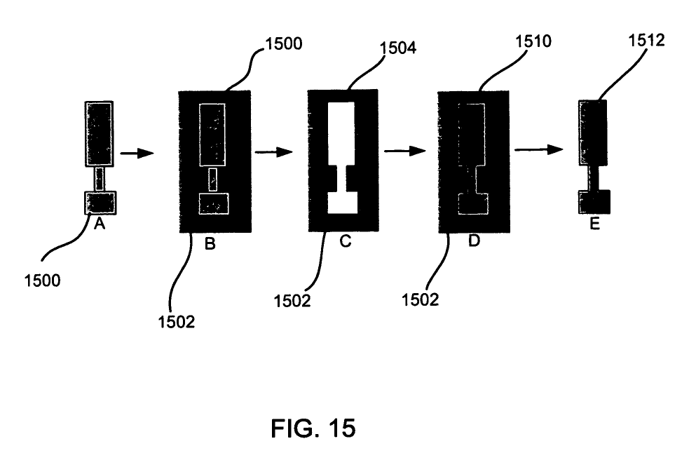

According to such embodiments, as shown in FIG. 15, steps A-E, a mold is fabricated by taking an object such as a bone 1500 and encapsulating the object in a curable matrix 1502 such as liquid PDMS precursors. Next, the curable matrix 1502 is cured. The bone 1500 is then removed, leaving a mold 1504 in a shape that corresponds to the molded object 1500. In some embodiments, the cured mold can be reversibly swelled to assist in removal of the object. Next, mold 1502 can be filled with dual cure materials of the present invention, such as for example, dual cure liquid PFPE precursors 1510. The dual cure material 1510 is then subjected to a first cure (e.g., thermal curing) to form a replicate object 1512 in the shape of bone 1500. Next, the replicate object 1512 can be implanted into the body as a replacement component. During implantation replicate object 1512 can be adhered to natural tissues, such as articular cartilage, portions of remaining natural bone, ligaments, tendons, other artificial joint components, and the like, by positioning the tissues with respect to replicate object 1512 and subjecting the combination to a second cure (e.g., photo curing).

According to other embodiments, dual cure materials of the present invention can be used in various cardiovascular applications and in other intraluminal applications. In some of these embodiments, the materials can be used to fabricate and/or augment body lumens, and to form artificial lumens (e.g., artificial blood vessels). The dual cure materials of the present invention can be molded, as shown in FIG. 15, to form replacement blood vessels for replacing damaged and/or occluded vessels within a body. Not only can the materials disclosed herein serve as conduits for blood flow, but they also can allow for diffusion of oxygen and nutrients through the vessel wall into surrounding tissues thus functioning much like a normal healthy blood vessel.

According to embodiments of the present invention, a method of replacing, in situ, a portion of a blood vessel includes injecting an oxygen permeable, bacterial impermeable dual cure liquid PFPE material into a lumen of a portion of a blood vessel such that the dual cure liquid PFPE coats the luminal surface of the blood vessel. The dual cure liquid PFPE is then subjected to a first cure technique to form an artificial blood vessel within the natural blood vessel. The biologic blood vessel can then be removed from the first cured PFPE material and the material can be subjected to a second cure or can be treated with another layer of dual curable liquid PFPE and the combination can be subjected to a second cure. Also, the second cure can be applied when the artificial blood vessel is positioned within the subject and activated to bind the material to the natural blood vessel. A working replacement for the blood vessel portion is thereby produced.

Embodiments of the present invention are particularly advantageous regarding repair and/or replacement of blood vessels. Given their high oxygen carrying ability and permeability, artificial vessels formed from PFPE materials have highly functional properties with synthetic vasavasorum characteristics. PFPE materials allow diffusion of oxygen through the walls and into surrounding dependent tissues, allow diffusion of sustaining nutrients, and diffusion of metabolites. PFPE materials substantially mimic vessels mechanically as they are flexible and compliant. Moreover, embodiments of the present invention are particularly suitable for use in heart by-pass surgery and as artificial arterio-venous shunts. PFPE materials can also be used to repair natural or synthetic arterio-venous shunts by coating the inside surface of the damaged or worn vessel and curing as described herein. According to other embodiments, intraluminal prostheses can be employed in sites of a body such as, but not limited to, biliary tree, esophagus, bowel, tracheo-bronchial tree, urinary tract, and the like.

In another embodiment, the dual cure materials of the present invention can be used to fabricate stents for repairing vascular tissue. In some embodiments the dual cure liquid material can be locally implanted and cured during a balloon angioplasty procedure, or the like, and subjected to a curing or a second curing after being locally positioned. In such embodiments, the dual cure liquid material can be first cured to form a manipulable sheet or tube of material. The manipulability of the sheet facilitates implantation of the stent precursor material. The stent precursor material can then be positioned, for example, by an angioplasty procedure. Upon positioning the stent precursor the implantation device can subject the stent precursor material to a second curing, thereby, creating a mechanically viable stent.

The dual cure PFPE materials, according to embodiments of the present invention, may be used with all of the cardiovascular and intraluminal devices described herein. PFPE materials may be utilized in the material(s) of these devices and/or may be provided as a coating on these devices.

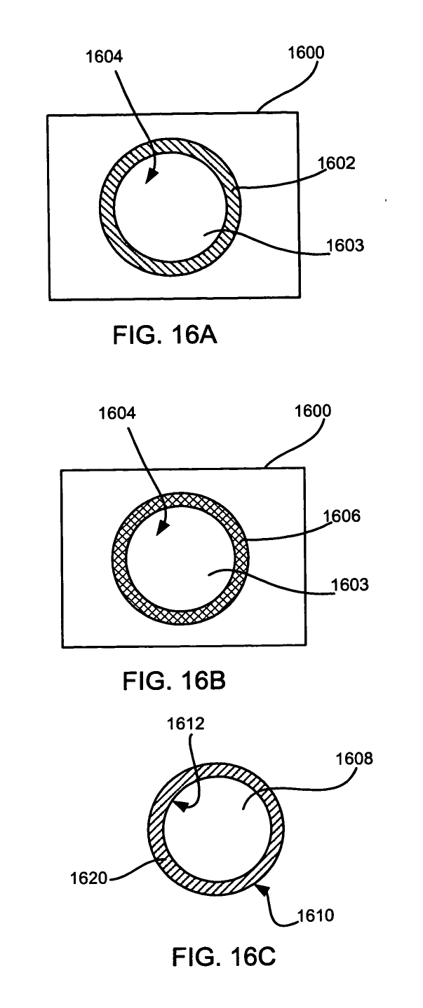

According to other embodiments and as shown in FIGS. 16A-16C, a biologic structure having a lumen (e.g., for example, a blood vessel) can be replaced with a medical device molded from the dual cure materials disclosed herein. FIG. 16 shows a top view or end view of a biologic structure 1602 having a lumen 1604. First, in molding the replacement vessel, lumen 1604 is filled with a temporary filler 1603. Temporary filler 1603 can be PDMS, foam, or another suitable material that can be inserted into vessel 1602. Filler 1603 can be administered into lumen 1604 such that a desired pressure is applied to the walls of vessel 1602. The pressure applied to the walls of vessel 1602 can be a pressure to mimic a biologic condition, a pressure below a normal biologic condition, a pressure above a normal biologic condition, a desired pressure, or the like. Vessel 1602 is then encapsulated into curable outer matrix 1600, such as for example liquid PDMS. Next, outer matrix 1600 is cured such that vessel 1602 is sandwiched between outer matrix 1600 and filler 1603.

Referring now to FIG. 16B, vessel 1602 is removed from between outer matrix 1600 and filler 1603, creating an receiving space 1606. Next, replacement material (e.g., liquid PFPE or the like) having dual cure capabilities, as described herein, is delivered into receiving space 1606. Replacement material can be injected, poured, sprayed, or the like into receiving space 1606. Next, replacement material is subjected to a first cure (e.g., photo or thermal curing) such that it solidifies, at least partially, and forms replacement device 1620. Following the first cure, outer matrix 1600 and filler 1603 are removed, thereby, leaving replacement device 1620 (FIG. 16C). Replacement device 1620 has an outer surface 1610, an inner surface 1612, and includes the characteristics of the natural biologic structure from which it was molded. Furthermore, replacement device 1620 includes a lumen 1608 that mimics the lumen of the biologic structure that replacement device 1620 was molded from.

Next, replacement device 1620 is positioned into the subject in the position where the natural component was removed or any other suitable implant location. Replacement device 1620 is aligned with biologic structures that it is configured to adhere to and function with and replacement device 1620 is treated with a second cure (e.g., thermal or photo curing). The second cure activates components of the replacement device (described herein) which in turn bind with the surrounding biologic tissue, thereby implanting and affixing replacement device 1620 with the subject. In other embodiments, replacement device 1620 can be bound to a bio-active polymer that is known to adhere to tissue, in the second curing step, such that the replacement device 1620 can bind to the biologic tissue through the bioactive polymer.

According to another embodiment, the dual cure material can be used to form a rigid structure that augments structural support to a skeletal portion of the subject. For example, damage to be augmented can be a crack or other defect in a bone. In some embodiments, the dual cure liquid material can be first molded or formed in vitro and first cured to form a structure of desired configuration. Next, the first cured structure can be implanted and positioned with respect to the damaged biologic structure to be augmented. Once in position, the first cured material can be treated with a second cure to further solidify and/or bond to the biologic structure. The dual cure mechanism of the present invention facilitates implantation of the structure because upon first curing the structure can retain a specific shape but be very compliant. The compliant nature of the structure after the first cure can reduce trauma inflicted on a patient while implanting the structure. Upon the second curing, the structure binds with the adjacent tissue or biologic component, seals the crack, and provides structural support to the damaged biological component. The composition and degree of curing of the implanted material can be altered to render a structure that resembles a desired functionality, such as strength, flexibility, rigidity, elasticity, combinations thereof and the like. Accordingly, the dual cured, flexible material may replace portions of ligaments, tendons, cartilage, muscles, and the like as well as tissue (e.g., flexible tissues) within the body of a subject.

In still further embodiments, the dual cure materials of the present invention can be utilized to form other medical devices, implant devices, biological replacement devices, medical procedure tools, surface treatments, combinations thereof, and the like. According to other embodiments, the dual cure materials of the present invention can be useful with the medical devices disclosed in published U.S. patent application no. 2005/0142315, including the publications cited therein, all of which are incorporated herein by reference in their entirety.

In still further embodiments, the dual cure materials disclosed and described herein can be used to form a patterned surface characteristic on the surface of medical devices. The patterned surface characteristic can provide useful properties to medical devices and as medical device coatings. The surface patterning of medical devices and medical implants can provide superhydrophobic coatings that can be extremely non-wetting to fluids. The patterned surfaces can also be highly resistant to biological fouling. Dual cure materials can be patterned by pouring a liquid precursor of the dual cure material onto a patterned template (e.g., silicon wafer) or by photolithography, and treating the precursor to a first curing, whereby the material solidifies or partially solidifies and takes the shape of the pattern on the patterned wafer. In some embodiments, the pattern can have structures that are between about 1 nm and about 500 nm. In other embodiments, the pattern can have structures that are between about 1 μm and 10 μm. In one embodiment the pattern is a repeated diamond shape pattern.

Next, the first cured material is released from the wafer to yield a patterned layer. Such a layer can then be used directly as a medical device or can be adhered, through a second curing, to other objects by the orthogonal curing methods previously described, thereby coating the surface of medical devices and implants and resulting in decreased wetability and decreased likelihood of bio-fouling of the medical device or implant.

In other embodiments, the dual cure materials are useful in dermatological applications including, for example, bandages, dressings, wound healing applications, burn care, reconstructive surgery, surgical glue, sutures, and the like. Because PFPE materials are oxygen permeable and bacterial impermeable, tissue underlying a PFPE bandage can receive oxygen while being protected against the ingress of dirt, microbial organisms, pathogens, and other forms of contamination and toxicity. In addition, the oxygen permeability and carrying capacity of PFPE materials can also help with preventing necrosis of healthy tissue under bandages and dressings, or under an area being treated.

According to an embodiment of the present invention, a method of applying “instant skin” to the body of a subject includes applying an oxygen permeable, bacterial impermeable liquid dual cure PFPE material onto a portion of the body of a subject. The dual cure PFPE material can be treated with a first cure to form layers of an approximate predetermined size and/or shape. After the first cure, the layered PFPE dual cure material is placed on the damaged zone of the patient. The dual cure PFPE is then subjected to a second cure such that the dual cure PFPE adheres to the patient and provides a oxygen permeable, microbial impermeable, waterproof, flexible, elastic, biocompatible artificial skin layer.

According to further embodiments, ocular implants and contact lenses can be formed from the dual cure materials of the present invention. These devices are advantageous over conventional ocular implants and contact lenses because the PFPE material is permeable to oxygen and resistant to bio-fouling. In addition, because of the lower surface energy, there is more comfort to the wearer as a result of the low friction generated the PFPE. In addition, the refractive index of PFPE materials can be adjusted for optimum performance for ocular implants and contact lenses. Further embodiments include cochlear implants utilizing the dual cure PFPE material. Using dual cure PFPE materials, tissue in-growth can be minimized, thus making removal of the device safer and less traumatic.

XIII. Other Applications

According to other embodiments of the present invention, traditional applications for silicone can be improved with the materials and methods of the present invention and according to further embodiments the applications can be replaced with the materials and methods disclosed herein. Silicone applications to which the materials and methods of the present invention are applicable include mold release agents, release layers, respiratory masks, anti-graffiti paint systems; aqueous coatings, sealants, mechanically assembled monolayers, micro plates & covers, tubing, water repellant, and organic solvent repellant.

Microextraction is a further application to which the materials and methods of the present invention can be applied. For example, the materials and methods of the present invention can be applied to substitute for or enhance the current techniques and chemicals used in microextraction. An example of microextraction is detailed in an article in Analytical Chemistry [69(6), 1197-1210, 1997] in which the authors placed 80 microliter chips of OV-1 extraction medium [poly(dimethylsiloxane)] in 50 ml flasks with 49 ml of aqueous sample, shook the flasks for 45 to 100 minutes, removed the chips, and placed them in the cell of a Shimadzu UV-260 spectrophotometer (Shimadzu Corp., Kyoto, Japan) to obtain a UV spectrum. Further described is a preconcentration by SPME that enables UV absorption spectroscopy to identify benzene at detection limits of 97 ppb, naphthalene at 0.40 ppb, 1-methylnaphthalene at 0.41 ppb, and 8 other aromatics at 5.5-12 ppb. In tests of samples spiked with unleaded gasoline, JP4 jet fuel, and no. 1 diesel fuel, preconcentration permits direct quantitation of dilute levels of aromatic species in aqueous samples without interference from humic substances in solution.

According to other embodiments, an application of the present invention can include substituting the materials and methods of the present invention for traditional chromatographic separation material. According to yet another embodiment of the present invention the materials and methods of the present invention can be combined with typical chromatographic separation materials. Chromatographic separations useful with the present invention are described in the following studies, incorporated herein by reference, and which describe that natural enantiomeric distribution of terpene alcohols on various natural matrices determined that, although distinctive for each matrix, the distribution is widely differentiated. While there is data available on the free bound linalool content in muscat wines, no data is available on the enantiomeric distribution of the same terpene alcohols in these wines. Researchers at DIFCA (Sezione di Chimica Analitica Argoali-mentare ed. Ambientale, Universita degli Studi di Milano, Via Celoria 2, 20133 Milan, Italy; Tel: 39 2 26607227, Fax: 39 2 2663057) have characterized muscat wines using gas chromatography (GC) chiral analysis. To determine the aromatic fraction of muscat wines, the enantiomeric excess of linalool and α-terpineol must be measured. F. Tateo and M. Bononi used two different fibers for the solid phase microextraction (SPME), one apolar (100 micron non-bonded polydimethylsiloxane) and one polar (partially crosslinked 65 micron carbowax/divinylbenzene). There was greater adsorption of the linalool using the polar fiber. The enantiomeric distribution of the linalool and of the α-terpineol were within fairly narrow limits and were considered characteristic indices. In order to assess the selectivity of SPME adsorption of the polar fiber with respect to a number of molecules, comparison was made using data obtained by direct injection. Greater sensitivity for the molecules was obtained using this technique.

In other applications, the materials and methods of the present invention can be substituted for PDMS materials used in outdoor capacities, such as for example, PDMS shed materials used to cover high voltage outdoor insulators. According to further embodiments, the presently disclosed materials and methods can be combined with PDMS materials used in outdoor capacities, such as for example, the high voltage outdoor insulator sheds described above. It is important that the surface of the shed of the insulator remains hydrophobic throughout its services life. It is known, however, that electrical discharges lead to an oxidation of the traditional surface and a temporary loss of hydrophobicity. According to a study of traditional materials, crosslinked polydimethylsiloxane (PDMS) containing Irganox 1076, Tinuvin 770 or Irganox 565 (Ciba Specialty Chemicals Corp., Tarrytown, N.Y., United States of America), prepared by swelling PDMS in a solution of one of these stabilizers in n-hexane, was exposed to a corona discharge and the corona exposure time (t-crit) to form a brittle, silica-like layer was determined by optical microscopy. The critical corona exposure time showed a linear increase with increasing stabilizer concentration; Tinuvin 770 showed the highest efficiency and Irganox 1076 the lowest. The increase in t-crit on corona exposure of the stabilized samples with reference to the value for unstabilized PDMS was similar to that reported earlier for air plasma exposed samples. The efficiency of the stabilizers towards corona-induced surface oxidation of PDMS also was confirmed by X-ray photoelectron spectroscopy. As will be appreciated by one of ordinary skill in the art, however, traditional materials utilizing PDMS can be significantly improved by the addition or augmentation with the materials and methods of the present invention.

Microvalves actuated by paraffin, such as, for example, microvalves containing silicone-rubber seals actuated by heating and cooling of paraffin have been proposed for development as integral components of microfluidic systems. According to an embodiment of the present invention, the materials and methods of the present invention can be substituted for, or combined with the silicone-rubber seals of such devices as the disclosed microvalve materials, thereby, increasing the physical and chemical properties of such microvalves.

Scratch-free surfaces is yet a further application to which the materials and methods of the present invention can be applied. The materials and methods of the present invention can be substituted for or used to augment the traditional scratch-free surface materials to improve their physical and chemical properties. As an example, research from Dow Corning of Freeland, Mich., has shown that adding masterbatches to thermoplastic olefins (TPOs) improves scratch resistance of TPO components. The company's MB50-series of masterbatches are in carrier-resin formats containing 50% ultra-high molecular weight polydimethylsiloxane, a scratch-resisting and lubricating additive. The additive lowers the coefficient of friction at the surface of the molded part. Surface-modifying masterbatches are now utilized and developed for various applications, such as in the automotive sector where it is being used in consoles, airbags, door skins and exterior components. Substituting the materials and methods of the present invention, or combining the materials and methods of the present invention to such scratch-free surface materials can improve the scratch-resistance of the materials.

The materials and methods of the present invention also can be applied to materials and methods used in the fabrication of sensors. An example of applying the materials and methods of the present invention to the materials that are used to make sensors, such as for example, polymeric membrane paste compositions will be appreciated by one of ordinary skill in the art from the following. Advantageous polymeric membrane paste compositions include a polyurethane/hydroxylated poly(vinyl chloride) compound and a silicone-based compound in appropriate solvent systems to provide screen-printable pastes of the appropriate viscosity and thixotropy. For an ion sensor to be commercially acceptable, it must have qualities beyond just electrochemical performance. For a sensor to be cost effective, it must be reproducible using mass production systems. There must be common electrochemical response characteristics within the members of a batch fabricated group. If the sensors are not all substantially identical, they will each be characterized by different lifetimes and response characteristics, creating difficulties in the field, not the least of which is the added cost associated with recalibration of equipment whenever the sensor is changed. Polymeric membranes are in common use as transducers in solid-state chemical sensors, particularly because such membranes have high selectivity to the ion of interest and can be made selective to a wide range of ions using one or many readily available ionophores. One known technique for forming the membranes is solvent casting; a technique which originated with ion-selective electrode technology. In addition to being a rather tedious operation, particularly in view of the small size of the sensors, this production method yields very high losses. The thickness and shape of the membrane cannot be controlled, resulting in an unacceptable lack of sensor reproducibility. An objective of the research at the University of Michigan was to provide a simple and economical system for batch fabrication of solid-state ion-selective sensors. Their method consists of installing a mask on a semiconductor substrate, the mask having at least one aperture having a predetermined configuration which corresponds with a desired membrane configuration. A polymeric membrane paste is applied to the mask, and a squeegee is drawn across the mask to force the paste into the aperture and in communication with the semiconductor substrate. In one form, the mask is of a metallic material, which can be a stainless steel mesh coated with a photoreactive emulsion. In another form, the mask is a metal foil stencil. The membrane which ultimately is produced has a thickness which corresponds to that of the mask, between about 25 and about 250 microns. The membrane paste can be formed of a polyurethane with an effective portion of an hydroxylated poly(vinyl chloride) copolymer; a polyimide-based compound; a silicone-based compound, such as silanol-terminated polydimethylsiloxane with the resistance-reducing additive, CN-derivatized silicone rubber; or any other suitable polymeric material. Thus, it will be appreciated that the materials and methods of the present invention can be applied to the materials and processes for forming sensors, such as the polymeric membrane compositions, polyimide-based compounds, polydimethylsiloxane, and silicone rubber, for example.

In another further embodiment, the materials and methods of the present invention can be substituted for or can be used to augment the materials and methods used in sol-gel capillary microextraction. Typically, sol-gel technology involves the encapsulation of active ingredients in micro- and nano-sized matrices, often silica based matrices, as well as nanospheres. Sol-gel capillary microextraction (sol-gel CME), for example, is a viable solventless extraction technique for the preconcentration of trace analytes. Sol-gel-coated capillaries are often employed for the extraction and preconcentration of a wide variety of polar and nonpolar analytes. Two different types of sol-gel coatings are used for extraction: sol-gel poly(dimethylsiloxane) (PDMS) and sol-gel poly(ethylene glycol) (PEG). A gravity-fed sample dispensing unit can be used to perform the extraction. The analysis of the extracted analytes can be performed by gas chromatography (GC). The extracted analytes are transferred to the GC column via thermal desorption. For this, the capillary with the extracted analytes can be connected to the inlet end of the GC column using a two-way press-fit fused-silica connector housed inside the GC injection port. Desorption of the analytes from the extraction capillary can be performed by rapid temperature programming (at 100 degrees C./min) of the GC injection port. The desorbed analytes are transported down the system by the helium flow and further focused at the inlet end of the GC column maintained at 30 degrees C. Sol-gel PDMS capillaries are commonly used for the extraction of nonpolar and moderately polar compounds (such as, but not limited to, polycyclic aromatic hydrocarbons, aldehydes, ketones), while sol-gel PEG capillaries are used for the extraction of polar compounds (such as, but not limited to, alcohols, phenols, amines). For both polar and nonpolar analytes, the run-to-run and capillary-to-capillary relative standard deviation (RSD) values for GC peak areas often remain under about 6% and about 4%, respectively. Parts per quadrillion level detection limits are achieved by coupling sol-gel CME with gas chromatography/flame ionization detection (GC-FID). The use of thicker sol-gel coatings and longer capillary segments of larger diameter (or capillaries with sol-gel monolithic beds) often lead to further enhancement of the extraction sensitivity. As will be appreciated by one of ordinary skill in the art, that replacing or combining the matrices and nanospheres commonly used in sol-gel applications with the materials and methods of the present invention can improve the efficiency and effectiveness of sol-gel processes.

In alternative embodiments the materials and methods of the present invention can be applied to processes, such as process aid for plastics and membrane separating processes. An example of membrane separating processes applicable with the present invention is described in Membrane & Separation Technology News, v. 15:no. 6, Feb. 1, 1997 (ISSN-0737-8483)).

Other silicone related arts that the methods and materials of the present invention are capable of augmenting or replacing include, but are not limited to, the disclosures in U.S. Pat. Nos. 6,887,911; 6,846,479; 6,808,814; 6,806,311; 6,804,062; 6,803,103; 6,797,740; the disclosures in U.S. Patent Application Nos. 2005/0147768; 2005/0112385; 2005/0111776; 2005/0091836; 2005/0052754; and the disclosure in EP1533339A1, each of which are incorporated by reference herein in their entirety.

The materials and methods disclosed in the following patent application can be used in the novel materials, methods, and devices of the present invention, including U.S. provisional patent application No. 60/706,786, filed Aug. 9, 2005; U.S. provisional patent application No. 60/732,727, filed Nov. 2, 2005; U.S. provisional patent application No. 60/799,317, filed May 10, 2006; PCT International Patent Application no. PCT/US05/04421, filed Feb. 14, 2005; and U.S. provisional patent application No. 60/544,905, filed Feb. 13, 2004, each of which is incorporated herein by reference in its entirety.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter.

### General Considerations

A PFPE microfluidic device has been previously reported by Rolland. J. et al. JACS 2004, 126, 2322-2323, which is incorporated herein by reference in its entirety. The specific PFPE material disclosed in Rolland, J. et al., was not chain extended and therefore did not possess the multiple hydrogen bonds that are present when PFPEs are chain extended with a diisocyanate linker. Nor did the material posses the higher molecular weights between crosslinks that are needed to improve mechanical properties such as modulus and tear strength which are critical to a variety of microfluidics applications. Furthermore, this material was not functionalized to incorporate various moieties, such as a charged species, a biopolymer, or a catalyst.

Herein is described a variety of materials and methods that improve medical devices, medical device fabrication techniques, medical device life span, tissue/device interfaces, anti-fouling of devices, and the like. Included in these improvements are methods which describe chain extension, improved adhesion to multiple PFPE layers and to other substrates such as glass, silicon, quartz, and other polymers as well as the ability to incorporate functional monomers capable of changing wetting properties or of attaching catalysts, biomolecules or other species. Also described are improved methods of curing PFPE elastomers which involve thermal free radical cures, two-component curing chemistries, and photocuring using photoacid generators.

### Example 1

A liquid PFPE precursor having the structure shown below (where n=2) is blended with 1 wt % of a free radical photoinitiator and poured over a microfluidics master containing 100-μm features in the shape of channels. A PDMS mold is used to contain the liquid in the desired area to a thickness of about 3 mm. The wafer is then placed in a UV chamber and exposed to UV light (λ=365) for 10 minutes under a nitrogen purge. Separately, a second master containing 100-μm features in the shape of channels is spin coated with a small drop of the liquid PFPE precursor over top of it at 3700 rpm for 1 minute to a thickness of about 20 μm. The wafer is then placed in a UV chamber and exposed to UV light (λ=365) for 10 minutes under a nitrogen purge. Thirdly, a smooth, flat PFPE layer is generated by drawing a doctor's blade across a small drop of the liquid PFPE precursor across a glass slide. The Slide is then placed in a UV chamber and exposed to UV light (λ=365) for 10 minutes under a nitrogen purge. The thicker layer is then removed, trimmed, and inlet holes are punched through it using a luer stub. The layer is then placed on top of the 20-μm thick layer and aligned in the desired area to form a seal. The layers are then placed in an oven and allowed to heat at 120° C. for 2 hours. The thin layer is then trimmed and the adhered layers are lifted from the master. Fluid inlet holes and outlet holes are punched using a luer stub. The bonded layers are then placed on the fully cured PFPE smooth layer on the glass slide and allowed to heat at 120° C. for 15 hours. Small needles can then be placed in the inlets to introduce fluids and to actuate membrane valves as reported by Unger M. et al. Science. 2000, 288, 113-6.

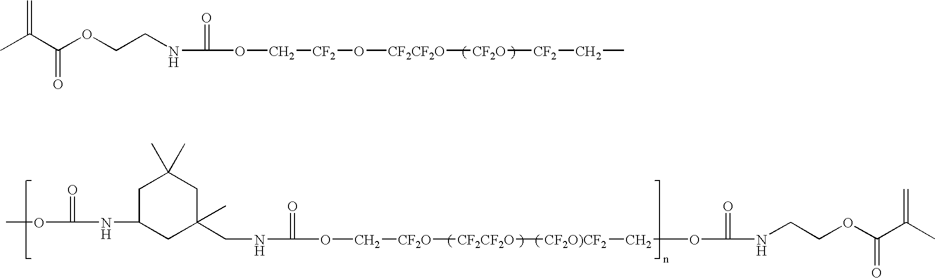

### Example 2

### Thermal Free Radical Glass

A liquid PFPE precursor encapped with methacrylate groups is blended with 1 wt % of 2,2-Azobisisobutyronitrile and poured over a microfluidics master containing 100-μm features in the shape of channels. A PDMS mold is used to contain the liquid in the desired area to a thickness of about 3 mm. The wafer is then placed in an oven at 65° C. for 20 hours under nitrogen purge. The cured layer is then removed, trimmed, and inlet holes are punched through it using a luer stub. The layer is then placed on top of a clean glass slide and fluids can be introduced through the inlet holes.

### Example 3

### Thermal Free Radical—Partial Cure Layer to Layer Adhesion

A liquid PFPE precursor encapped with methacrylate groups is blended with 1 wt % of 2,2-Azobisisobutyronitrile and poured over a microfluidics master containing 100-μm features in the shape of channels. A PDMS mold is used to contain the liquid in the desired area to a thickness of about 3 mm. The wafer is then placed in an oven at 65° C. for 2-3 hours under nitrogen purge. Separately, a second master containing 100-μm features in the shape of channels is spin coated with a small drop of the liquid PFPE precursor over top of it at 3700 rpm for 1 minute to a thickness of about 20 μm. The wafer is then placed in an oven at 65° C. for 2-3 hours under nitrogen purge. Thirdly, a smooth, flat PFPE layer is generated by drawing a doctor's blade across a small drop of the liquid PFPE precursor across a glass slide. The wafer is then placed in an oven at 65° C. for 2-3 hours under nitrogen purge. The thicker layer is then removed, trimmed, and inlet holes are punched through it using a luer stub. The layer is then placed on top of the 20-μm thick layer and aligned in the desired area to form a seal. The layers are then placed in an oven and allowed to heat at 65° C. for 10 hours. The thin layer is then trimmed and the adhered layers are lifted from the master. Fluid inlet holes and outlet holes are punched using a luer stub. The bonded layers are then placed on the partially cured PFPE smooth layer on the glass slide and allowed to heat at 65° C. for 10 hours. Small needles can then be placed in the inlets to introduce fluids and to actuate membrane valves as reported by Unger, M. et al. Science. 2000, 288, 113-6.

### Example 4

### Thermal Free Radical—Partial Cure Adhesion to Polyurethane

A photocurable liquid polyurethane precursor containing methacrylate groups is blended with 1 wt % of 2,2-Azobisisobutyronitrile and poured over a microfluidics master containing 100-μm features in the shape of channels. A PDMS mold is used to contain the liquid in the desired area to a thickness of approximately 3 mm. The wafer is then placed in an oven at 65° C. for 2-3 hours under nitrogen purge. Separately, a second master containing 100-μm features in the shape of channels is spin coated with a small drop of the liquid PFPE precursor over top of it at 3700 rpm for 1 minute to a thickness of approximately 20 μm. The wafer is then placed in an oven at 65° C. for 2-3 hours under nitrogen purge. Thirdly, a smooth, flat PFPE layer is generated by drawing a doctor's blade across a small drop of the liquid PFPE precursor across a glass slide. The wafer is then placed in an oven at 65° C. for 2-3 hours under nitrogen purge. The thicker layer is then removed, trimmed, and inlet holes are punched through it using a luer stub. The layer is then placed on top of the 20-μm thick layer and aligned in the desired area to form a seal. The layers are then placed in an oven and allowed to heat at 65° C. for 10 hours. The thin layer is then trimmed and the adhered layers are lifted from the master. Fluid inlet holes and outlet holes are punched using a luer stub. The bonded layers are then placed on the partially cured PFPE smooth layer on the glass slide and allowed to heat at 65° C. for 10 hours. Small needles can then be placed in the inlets to introduce fluids and to actuate membrane valves as reported by Unger, M. et al. Science. 2000, 288, 113-6.

### Example 5

### Thermal Free Radical—Partial Cure Adhesion to Silicone-Containing Polyurethane

A photocurable liquid polyurethane precursor containing PDMS blocks and methacrylate groups is blended with 1 wt % of 2,2-Azobisisobutyronitrile and poured over a microfluidics master containing 100-μm features in the shape of channels. A PDMS mold is used to contain the liquid in the desired area to a thickness of approximately 3 mm. The wafer is then placed in an oven at 65° C. for 2-3 hours under nitrogen purge. Separately, a second master containing 100-μm features in the shape of channels is spin coated with a small drop of the liquid PFPE precursor over top of it at 3700 rpm for 1 minute to a thickness of approximately 20 μm. The wafer is then placed in an oven at 65° C. for 2-3 hours under nitrogen purge. Thirdly, a smooth, flat PFPE layer is generated by drawing a doctor's blade across a small drop of the liquid PFPE precursor across a glass slide. The wafer is then placed in an oven at 65° C. for 2-3 hours under nitrogen purge. The thicker layer is then removed, trimmed, and inlet holes are punched through it using a luer stub. The layer is then placed on top of the 20-μm thick layer and aligned in the desired area to form a seal. The layers are then placed in an oven and allowed to heat at 65° C. for 10 hours. The thin layer is then trimmed and the adhered layers are lifted from the master. Fluid inlet holes and outlet holes are punched using a luer stub. The bonded layers are then placed on the partially cured PFPE smooth layer on the glass slide and allowed to heat at 65° C. for 10 hours. Small needles can then be placed in the inlets to introduce fluids and to actuate membrane valves as reported by Unger. M. et al. Science. 2000, 288, 113-6.

### Example 6

### Thermal Free Radical—Partial Cure Adhesion to PFPE-PDMS Block Copolymer

A liquid precursor containing both PFPE and PDMS blocks encapped with methacrylate groups is blended with 1 wt % of 2,2-Azobisisobutyronitrile and poured over a microfluidics master containing 100-μm features in the shape of channels. A PDMS mold is used to contain the liquid in the desired area to a thickness of approximately 3 mm. The wafer is then placed in an oven at 65° C. for 2-3 hours under nitrogen purge. Separately, a second master containing 100-μm features in the shape of channels is spin coated with a small drop of the liquid PFPE precursor over top of it at 3700 rpm for 1 minute to a thickness of approximately 20 μm. The wafer is then placed in an oven at 65° C. for 2-3 hours under nitrogen purge. Thirdly, a smooth, flat PFPE layer is generated by drawing a doctor's blade across a small drop of the liquid PFPE precursor across a glass slide. The wafer is then placed in an oven at 65° C. for 2-3 hours under nitrogen purge. The thicker layer is then removed, trimmed, and inlet holes are punched through it using a luer stub. The layer is then placed on top of the 20-μm thick layer and aligned in the desired area to form a seal. The layers are then placed in an oven and allowed to heat at 65° C. for 10 hours. The thin layer is then trimmed and the adhered layers are lifted from the master. Fluid inlet holes and outlet holes are punched using a luer stub. The bonded layers are then placed on the partially cured PFPE smooth layer on the glass slide and allowed to heat at 65° C. for 10 hours. Small needles can then be placed in the inlets to introduce fluids and to actuate membrane valves as reported by Unger, M. et al. Science. 2000, 288, 113-6.

### Example 7

### Thermal Free Radical—Partial Cure Glass Adhesion

A liquid PFPE precursor encapped with methacrylate groups is blended with 1 wt % of 2,2-Azobisisobutyronitrile and poured over a microfluidics master containing 100-μm features in the shape of channels. A PDMS mold is used to contain the liquid in the desired area to a thickness of about 3 mm. The wafer is then placed in an oven at 65° C. for 2-3 hours under nitrogen purge. The partially cured layer is removed from the wafer and inlet holes are punched using a luer stub. The layer is then placed on top of a glass slide treated with a silane coupling agent, trimethoxysilyl propyl methacrylate. The layer is then placed in an oven and allowed to heat at 65° C. for 20 hours, permanently bonding the PFPE layer to the glass slide. Small needles can then be placed in the inlets to introduce fluids.

### Example 8

### Thermal Free Radical—Partial Cure PDMS Adhesion

A liquid poly(dimethylsiloxane) precursor poured over a microfluidics master containing 100-μm features in the shape of channels. The wafer is then placed in an oven at 80° C. for 3 hours. Separately, a second master containing 100-μm features in the shape of channels is spin coated with a small drop of liquid PFPE precursor encapped with methacrylate units at 3700 rpm for 1 minute to a thickness of about 20 μm. The wafer is then placed in an oven at 65° C. for 2-3 hours under nitrogen purge. The PDMS layer is then removed, trimmed, and inlet holes are punched through it using a luer stub. The layer is then treated with an oxygen plasma for 20 minutes followed by treatment with a silane coupling agent, trimethoxysilyl propyl methacrylate. The treated PDMS layer is then placed on top of the partially cured PFPE thin layer and heated at 65° C. for 10 hours. The thin layer is then trimmed and the adhered layers are lifted from the master. Fluid inlet holes and outlet holes are punched using a luer stub. The bonded layers are then placed on the partially cured PFPE smooth layer on the glass slide and allowed to heat at 65° C. for 10 hours. Small needles can then be placed in the inlets to introduce fluids and to actuate membrane valves as reported by Unger, M. et al. Science. 2000, 288, 113-6.

### Example 9

### Thermal Free Radical PDMS Adhesion Using SYLGARD 184® and Functional PDMS

A liquid poly(dimethylsiloxane) precursor is designed such that it can be part of the base or curing component of SYLGARD 184®. The precursor contains latent functionalities such as epoxy, methacrylate, or amines and is mixed with the standard curing agents and poured over a microfluidics master containing 100-μm features in the shape of channels. The wafer is then placed in an oven at 80° C. for 3 hours. Separately, a second master containing 100-μm features in the shape of channels is spin coated with a small drop of liquid PFPE precursor encapped with methacrylate units at 3700 rpm for 1 minute to a thickness of approximately 20 μm. The wafer is then placed in an oven at 65° C. for 2-3 hours under nitrogen purge. The PDMS layer is then removed, trimmed, and inlet holes are punched through it using a luer stub. The PDMS layer is then placed on top of the partially cured PFPE thin layer and heated at 65° C. for 10 hours. The thin layer is then trimmed and the adhered layers are lifted from the master. Fluid inlet holes and outlet holes are punched using a luer stub. The bonded layers are then placed on the partially cured PFPE smooth layer on the glass slide and allowed to heat at 65° C. for 10 hours. Small needles can then be placed in the inlets to introduce fluids and to actuate membrane valves as reported by Unger, M. et al. Science. 2000, 288, 113-6.

### Example 10

### Epoxy/Amine

A two-component liquid PFPE precursor system such as shown below containing a PFPE diepoxy and a PFPE diamine are blended together in a stochiometric ratio and poured over a microfluidics master containing 100-μm features in the shape of channels. A PDMS mold is used to contain the liquid in the desired area to a thickness of about 3 mm. The wafer is then placed in an oven at 65° C. for 5 hours. The cured layer is then removed, trimmed, and inlet holes are punched through it using a luer stub. The layer is then placed on top of a clean glass slide and fluids can be introduced through the inlet holes.

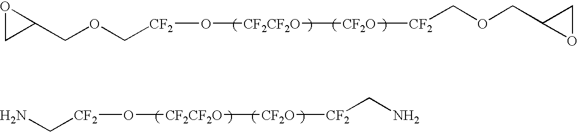

### Example 11

### Epoxy/Amine—Excess Adhesion to Glass

A two-component liquid PFPE precursor system such as shown below containing a PFPE diepoxy and a PFPE diamine are blended together in a 4:1 epoxy:amine ratio such that there is an excess of epoxy and poured over a microfluidics master containing 100-μm features in the shape of channels. A PDMS mold is used to contain the liquid in the desired area to a thickness of about 3 mm. The wafer is then placed in an oven at 65° C. for 5 hours. The cured layer is then removed, trimmed, and inlet holes are punched through it using a luer stub. The layer is then placed on top of a clean glass slide that has been treated with a silane coupling agent, aminopropyltriethoxy silane. The slide is then heated at 65° C. for 5 hours to permanently bond the device to the glass slide. Fluids can then be introduced through the inlet holes.

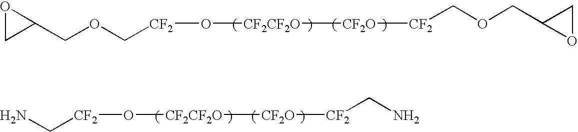

### Example 12

### Epoxy/Amine—Excess Adhesion to PFPE Layers

A two-component liquid PFPE precursor system such as shown below containing a PFPE diepoxy and a PFPE diamine are blended together in a 1:4 epoxy:amine ratio such that there is an excess of amine and poured over a microfluidics master containing 100-μm features in the shape of channels. A PDMS mold is used to contain the liquid in the desired area to a thickness of about 3 mm. Separately, a second master containing 100-μm features in the shape of channels is coated with a small drop of liquid PFPE precursors blended in a 4:1 epoxy:amine ratio such that there is an excess of epoxy units and spin coated at 3700 rpm for 1 minute to a thickness of about 20 μm. The wafer is then placed in an oven at 65° C. for 5 hours. The thick layer is then removed, trimmed, and inlet holes are punched through it using a luer stub. The thick layer is then placed on top of the cured PFPE thin layer and heated at 65° C. for 5 hours. The thin layer is then trimmed and the adhered layers are lifted from the master. Fluid inlet holes and outlet holes are punched using a luer stub. The bonded layers are then placed on a glass slide treated with a silane coupling agent, aminopropyltriethoxy silane and heated in an oven at 65° C. for 5 hours to adhere the device to the glass slide. Small needles can then be placed in the inlets to introduce fluids and to actuate membrane valves as reported by Unger, M. et al. Science. 2000, 288, 113-6.

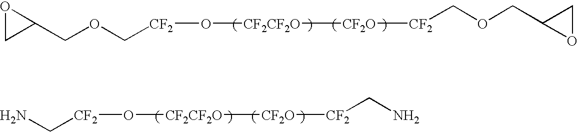

### Example 13

### Epoxy/Amine—Excess Adhesion to PDMS Layers

A liquid poly(dimethylsiloxane) precursor is poured over a microfluidics master containing 100-μm features in the shape of channels. The wafer is then placed in an oven at 80° C. for 3 hours. Separately, a second master containing 100-μm features in the shape of channels is coated with a small drop of liquid PFPE precursors blended in a 4:1 epoxy:amine ratio such that there is an excess of epoxy units and spin coated at 3700 rpm for 1 minute to a thickness of about 20 μm. The wafer is then placed in an oven at 65° C. for 5 hours. The PDMS layer is then removed, trimmed, and inlet holes are punched through it using a luer stub. The layer is then treated with an oxygen plasma for 20 minutes followed by treatment with a silane coupling agent, aminopropyltriethoxy silane. The treated PDMS layer is then placed on top of the PFPE thin layer and heated at 65° C. for 10 hours to adhere the two layers. The thin layer is then trimmed and the adhered layers are lifted from the master. Fluid inlet holes and outlet holes are punched using a luer stub. The bonded layers are then placed on a glass slide treated with aminopropyltriethoxy silane and allowed to heat at 65° C. for 10 hours. Small needles can then be placed in the inlets to introduce fluids and to actuate membrane valves as reported by Unger. M. et al. Science. 2000, 288, 113-6.

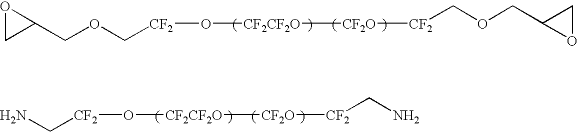

### Example 14

### Epoxy/Amine—Excess Adhesion to PFPE Layers, Attachment of a Biomolecule

A two-component liquid PFPE precursor system such as shown below containing a PFPE diepoxy and a PFPE diamine are blended together in a 1:4 epoxy:amine ratio such that there is an excess of amine and poured over a microfluidics master containing 100-μm features in the shape of channels. A PDMS mold is used to contain the liquid in the desired area to a thickness of about 3 mm. Separately, a second master containing 100-μm features in the shape of channels is coated with a small drop of liquid PFPE precursors blended in a 4:1 epoxy:amine ratio such that there is an excess of epoxy units and spin coated at 3700 rpm for 1 minute to a thickness of about 20 μm. The wafer is then placed in an oven at 65° C. for 5 hours. The thick layer is then removed, trimmed, and inlet holes are punched through it using a luer stub. The thick layer is then placed on top of the cured PFPE thin layer and heated at 65° C. for 5 hours. The thin layer is then trimmed and the adhered layers are lifted from the master. Fluid inlet holes and outlet holes are punched using a luer stub. The bonded layers are then placed on a glass slide treated with a silane coupling agent, aminopropyltriethoxy silane and heated in an oven at 65° C. for 5 hours to adhere the device to the glass slide. Small needles can then be placed in the inlets to introduce fluids and to actuate membrane valves as reported by Unger. M. et al. Science. 2000, 288, 113-6. An aqueous solution containing a protein functionalized with a free amine is then flowed through the channel which is lined with unreacted epoxy moieties, in such a way that the channel is then functionalized with the protein.

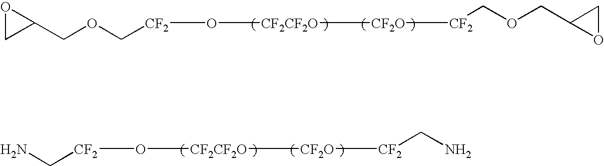

### Example 15

### Epoxy/Amine—Excess Adhesion to PFPE Layers, Attachment of a Charged Species

A two-component liquid PFPE precursor system such as shown below containing a PFPE diepoxy and a PFPE diamine are blended together in a 1:4 epoxy:amine ratio such that there is an excess of amine and poured over a microfluidics master containing 100-μm features in the shape of channels. A PDMS mold is used to contain the liquid in the desired area to a thickness of about 3 mm. Separately, a second master containing 100-μm features in the shape of channels is coated with a small drop of liquid PFPE precursors blended in a 4:1 epoxy:amine ratio such that there is an excess of epoxy units and spin coated at 3700 rpm for 1 minute to a thickness of about 20 μm. The wafer is then placed in an oven at 65° C. for 5 hours. The thick layer is then removed, trimmed, and inlet holes are punched through it using a luer stub. The thick layer is then placed on top of the cured PFPE thin layer and heated at 65° C. for 5 hours. The thin layer is then trimmed and the adhered layers are lifted from the master. Fluid inlet holes and outlet holes are punched using a luer stub. The bonded layers are then placed on a glass slide treated with a silane coupling agent, aminopropyltriethoxy silane and heated in an oven at 65° C. for 5 hours to adhere the device to the glass slide. Small needles can then be placed in the inlets to introduce fluids and to actuate membrane valves as reported by Unger, M. et al. Science. 2000, 288, 113-6. An aqueous solution containing a charged molecule functionalized with a free amine is then flowed through the channel which is lined with unreacted epoxy moieties, in such a way that the channel is then functionalized with the charged molecule.

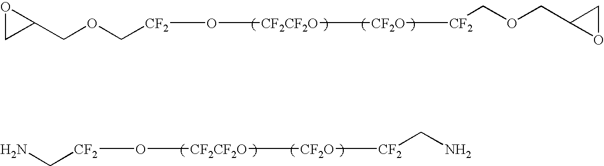

### Example 16

### Epoxy/Amine—Partial Cure Adhesion to Glass

A two-component liquid PFPE precursor system such as shown below containing a PFPE diepoxy and a PFPE diamine are blended together in a stochiometric ratio and poured over a microfluidics master containing 100-μm features in the shape of channels. A PDMS mold is used to contain the liquid in the desired area to a thickness of about 3 mm. The wafer is then placed in an oven at 65° C. for 0.5 hours such that it is partially cured. The partially cured layer is then removed, trimmed, and inlet holes are punched through it using a luer stub. The layer is then placed on a glass slide treated with a silane coupling agent, aminopropyltriethoxy silane, and allowed to heat at 65° C. for 5 hours such that it is adhered to the glass slide. Small needles can then be placed in the inlets to introduce fluids.

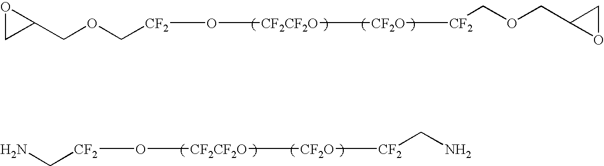

### Example 17

### Epoxy/Amine—Partial Cure Layer to Layer Adhesion

A two-component liquid PFPE precursor system such as shown below containing a PFPE diepoxy and a PFPE diamine are blended together in a stochiometric ratio and poured over a microfluidics master containing 100-μm features in the shape of channels. A PDMS mold is used to contain the liquid in the desired area to a thickness of about 3 mm. The wafer is then placed in an oven at 65° C. for 0.5 hours such that it is partially cured. The partially cured layer is then removed, trimmed, and inlet holes are punched through it using a luer stub. Separately, a second master containing 100-μm features in the shape of channels is spin coated with a small drop of the liquid PFPE precursors over top of it at 3700 rpm for 1 minute to a thickness of about 20 μm. The wafer is then placed in an oven at 65° C. for 0.5 hours such that it is partially cured. The thick layer is then placed on top of the 20-μm thick layer and aligned in the desired area to form a seal. The layers are then placed in an oven and allowed to heat at 65° C. for 1 hour to adhere the two layers. The thin layer is then trimmed and the adhered layers are lifted from the master. Fluid inlet holes and outlet holes are punched using a luer stub. The bonded layers are then placed on a glass slide treated with a silane coupling agent, aminopropyltriethoxy silane, and allowed to heat at 65° C. for 10 hours. Small needles can then be placed in the inlets to introduce fluids and to actuate membrane valves as reported by Unger. M. et al. Science. 2000, 288, 113-6.

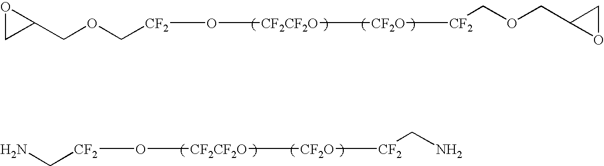

### Example 18

### Epoxy/Amine—Partial Cure PDMS Adhesion

A liquid poly(dimethylsiloxane) precursor is poured over a microfluidics master containing 100-μm features in the shape of channels. The wafer is then placed in an oven at 80° C. for 3 hours. The cured PDMS layer is then removed, trimmed, and inlet holes are punched through it using a luer stub. The layer is then treated with an oxygen plasma for 20 minutes followed by treatment with a silane coupling agent, aminopropyltriethoxy silane. Separately, a second master containing 100-μm features in the shape of channels is spin coated with a small drop of liquid PFPE precursors mixed in a stochiometric ratio at 3700 rpm for 1 minute to a thickness of about 20 μm. The wafer is then placed in an oven at 65° C. for 0.5 hours. The treated PDMS layer is then placed on top of the partially cured PFPE thin layer and heated at 65° C. for 1 hour. The thin layer is then trimmed and the adhered layers are lifted from the master. Fluid inlet holes and outlet holes are punched using a luer stub. The bonded layers are then placed on a glass slide treated with aminopropyltriethoxy silane and allowed to heat at 65° C. for 10 hours. Small needles can then be placed in the inlets to introduce fluids and to actuate membrane valves as reported by Unger, M. et al. Science. 2000, 288, 113-6.

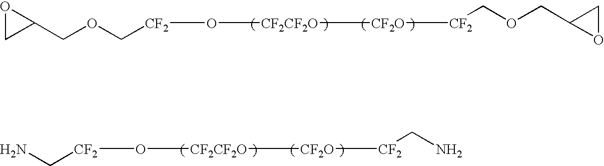

### Example 19

### Photocuring with Latent Functional Groups Available Post Cure Adhesion to Glass

A liquid PFPE precursor having the structure shown below (where R is an epoxy group, the curvy lines are PFPE chains, and the circle is a linking molecule) is blended with 1 wt % of a free radical photoinitiator and poured over a microfluidics master containing 100-μm features in the shape of channels. A PDMS mold is used to contain the liquid in the desired area to a thickness of about 3 mm. The wafer is then placed in a UV chamber and exposed to UV light (λ=365) for 10 minutes under a nitrogen purge. The fully cured layer is then removed from the master and inlet holes are punched using a luer stub. The device is placed on a glass slide treated with a silane coupling agent, aminopropyltriethoxy silane, and allowed to heat at 65° C. for 15 hours permanently bonding the device to the glass slide. Small needles can then be placed in the inlets to introduce fluids.

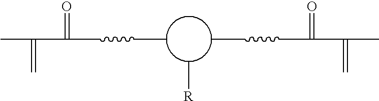

### Example 20

### Photocuring with Latent Functional Groups Available Post Cure Adhesion to PFPE

A liquid PFPE precursor having the structure shown below (where R is an epoxy group), the curvy lines are PFPE chains, and the circle is a linking molecule) is blended with 1 wt % of a free radical photoinitiator and poured over a microfluidics master containing 100-μm features in the shape of channels. A PDMS mold is used to contain the liquid in the desired area to a thickness of about 3 mm. The wafer is then placed in a UV chamber and exposed to UV light (λ=365) for 10 minutes under a nitrogen purge. The fully cured layer is then removed from the master and inlet holes are punched using a luer stub. Separately a second master containing 100-μm features in the shape of channels is spin coated with a small drop of the liquid PFPE precursor (where R is an amine group) over top of it at 3700 rpm for 1 minute to a thickness of about 20 μm. The wafer is then placed in a UV chamber and exposed to UV light (λ=365) for 10 minutes under a nitrogen purge. The thicker layer is then placed on top of the 20-μm thick layer and aligned in the desired area to form a seal. The layers are then placed in an oven and allowed to heat at 65° C. for 2 hours. The thin layer is then trimmed and the adhered layers are lifted from the master. Fluid inlet holes and outlet holes are punched using a luer stub. The bonded layers are then placed on a glass slide treated with a silane coupling agent, aminopropyltriethoxy silane, and allowed to heat at 65° C. for 15 hours permanently bonding the device to the glass slide. Small needles can then be placed in the inlets to introduce fluids and to actuate membrane valves as reported by Unger, M. et al. Science. 2000, 288, 113-6.

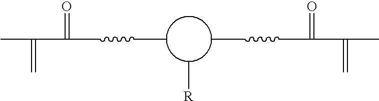

### Example 21

### Photocuring w/Latent Functional Groups Available Post Cure Adhesion to PDMS

A liquid poly(dimethylsiloxane) precursor is poured over a microfluidics master containing 100-μm features in the shape of channels. The wafer is then placed in an oven at 80° C. for 3 hours. The cured PDMS layer is then removed, trimmed, and inlet holes are punched through it using a luer stub. The layer is then treated with an oxygen plasma for 20 minutes followed by treatment with a silane coupling agent, aminopropyltriethoxy silane. Separately a second master containing 100-μm features in the shape of channels is spin coated with a small drop of the liquid PFPE precursor (where R is an epoxy) over top of it at 3700 rpm for 1 minute to a thickness of about 20 μm. The wafer is then placed in a UV chamber and exposed to UV light (λ=365) for 10 minutes under a nitrogen purge. The thicker PDMS layer is then placed on top of the 20-μm thick layer and aligned in the desired area to form a seal. The layers are then placed in an oven and allowed to heat at 65° C. for 2 hours. The thin layer is then trimmed and the adhered layers are lifted from the master. Fluid inlet holes and outlet holes are punched using a luer stub. The bonded layers are then placed on a glass slide treated with a silane coupling agent, aminopropyltriethoxy silane, and allowed to heat at 65° C. for 15 hours permanently bonding the device to the glass slide. Small needles can then be placed in the inlets to introduce fluids and to actuate membrane valves as reported by Unger. M. et al. Science. 2000, 288, 113-6.

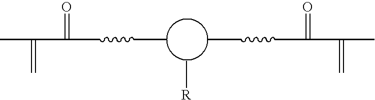

### Example 22

### Photocuring with Latent Functional Groups Available Post Cure Attachment of Biomolecule

A liquid PFPE precursor having the structure shown below (where R is an amine group), the curvy lines are PFPE chains, and the circle is a linking molecule) is blended with 1 wt % of a free radical photoinitiator and poured over a microfluidics master containing 100-μm features in the shape of channels. A PDMS mold is used to contain the liquid in the desired area to a thickness of about 3 mm. The wafer is then placed in a UV chamber and exposed to UV light (λ=365) for 10 minutes under a nitrogen purge. The fully cured layer is then removed from the master and inlet holes are punched using a luer stub. Separately a second master containing 100-μm features in the shape of channels is spin coated with a small drop of the liquid PFPE precursor (where R is an epoxy group) over top of it at 3700 rpm for 1 minute to a thickness of about 20 μm. The wafer is then placed in a UV chamber and exposed to UV light (λ=365) for 10 minutes under a nitrogen purge. The thicker layer is then placed on top of the 20-μm thick layer and aligned in the desired area to form a seal. The layers are then placed in an oven and allowed to heat at 65° C. for 2 hours. The thin layer is then trimmed and the adhered layers are lifted from the master. Fluid inlet holes and outlet holes are punched using a luer stub. The bonded layers are then placed on a glass slide treated with a silane coupling agent, aminopropyltriethoxy silane, and allowed to heat at 65° C. for 15 hours permanently bonding the device to the glass slide. Small needles can then be placed in the inlets to introduce fluids and to actuate membrane valves as reported by Unger. M. et al. Science. 2000, 288, 113-6. An aqueous solution containing a protein functionalized with a free amine is then flowed through the channel which is lined with unreacted epoxy moieties, in such a way that the channel is then functionalized with the protein.

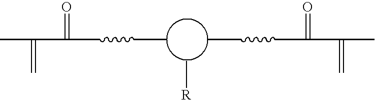

### Example 23

Photocuring with Latent Functional Groups Available Post Cure Attachment of Charged Species

A liquid PFPE precursor having the structure shown below (where R is an amine group), the curvy lines are PFPE chains, and the circle is a linking molecule) is blended with 1 wt % of a free radical photoinitiator and poured over a microfluidics master containing 100-μm features in the shape of channels. A PDMS mold is used to contain the liquid in the desired area to a thickness of about 3 mm. The wafer is then placed in a UV chamber and exposed to UV light (λ=365) for 10 minutes under a nitrogen purge. The fully cured layer is then removed from the master and inlet holes are punched using a luer stub. Separately a second master containing 100-μm features in the shape of channels is spin coated with a small drop of the liquid PFPE precursor (where R is an epoxy group) over top of it at 3700 rpm for 1 minute to a thickness of about 20 μm. The wafer is then placed in a UV chamber and exposed to UV light (λ=365) for 10 minutes under a nitrogen purge. The thicker layer is then placed on top of the 20-μm thick layer and aligned in the desired area to form a seal. The layers are then placed in an oven and allowed to heat at 65° C. for 2 hours. The thin layer is then trimmed and the adhered layers are lifted from the master. Fluid inlet holes and outlet holes are punched using a luer stub. The bonded layers are then placed on a glass slide treated with a silane coupling agent, aminopropyltriethoxy silane, and allowed to heat at 65° C. for 15 hours permanently bonding the device to the glass slide. Small needles can then be placed in the inlets to introduce fluids and to actuate membrane valves as reported by Unger. M. et al. Science. 2000, 288, 113-6. An aqueous solution containing a charged molecule functionalized with a free amine is then flowed through the channel which is lined with unreacted epoxy moieties, in such a way that the channel is then functionalized with the charged molecule.

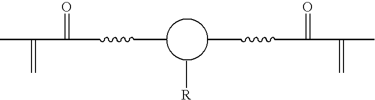

### Example 24

### Photocuring with Functional Monomers Available Post Cure Adhesion to Glass

A liquid PFPE dimethacrylate precursor or a monomethacrylate PFPE macromonomer is blended with a monomer having the structure shown below (where R is an epoxy group) and blended with 1 wt % of a free radical photoinitiator and poured over a microfluidics master containing 100-μm features in the shape of channels. A PDMS mold is used to contain the liquid in the desired area to a thickness of about 3 mm. The wafer is then placed in a UV chamber and exposed to UV light (λ=365) for 10 minutes under a nitrogen purge. The fully cured layer is then removed from the master and inlet holes are punched using a luer stub. The device is placed on a glass slide treated with a silane coupling agent, aminopropyltriethoxy silane, and allowed to heat at 65° C. for 15 hours permanently bonding the device to the glass slide. Small needles can then be placed in the inlets to introduce fluids.

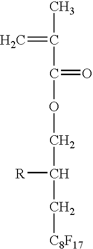

### Example 25

Photocuring with Functional Monomers Available Post Cure Adhesion to PFPE

A liquid PFPE dimethacrylate precursor is blended with a monomer having the structure shown below (where R is an epoxy group) and blended with 1 wt % of a free radical photoinitiator and poured over a microfluidics master containing 100-μm features in the shape of channels. A PDMS mold is used to contain the liquid in the desired area to a thickness of about 3 mm. The wafer is then placed in a UV chamber and exposed to UV light (λ=365) for 10 minutes under a nitrogen purge. The fully cured layer is then removed from the master and inlet holes are punched using a luer stub. Separately a second master containing 100-μm features in the shape of channels is spin coated with a small drop of the liquid PFPE precursor plus functional (where R is an amine group) over top of it at 3700 rpm for 1 minute to a thickness of about 20 μm. The wafer is then placed in a UV chamber and exposed to UV light (λ=365) for 10 minutes under a nitrogen purge. The thicker layer is then placed on top of the 20-μm thick layer and aligned in the desired area to form a seal. The layers are then placed in an oven and allowed to heat at 65° C. for 2 hours. The thin layer is then trimmed and the adhered layers are lifted from the master. Fluid inlet holes and outlet holes are punched using a luer stub. The bonded layers are then placed on a glass slide treated with a silane coupling agent, aminopropyltriethoxy silane, and allowed to heat at 65° C. for 15 hours permanently bonding the device to the glass slide. Small needles can then be placed in the inlets to introduce fluids and to actuate membrane valves as reported by Unger. M. et al. Science. 2000, 288, 113-6.

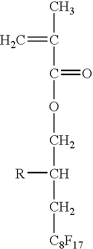

### Example 26

### Photocuring with Functional Monomers Available Post Cure Adhesion to PDMS

A liquid poly(dimethylsiloxane) precursor is poured over a microfluidics master containing 100-μm features in the shape of channels. The wafer is then placed in an oven at 80° C. for 3 hours. The cured PDMS layer is then removed, trimmed, and inlet holes are punched through it using a luer stub. The layer is then treated with an oxygen plasma for 20 minutes followed by treatment with a silane coupling agent, aminopropyltriethoxy silane. Separately a second master containing 100-μm features in the shape of channels is spin coated with a small drop of a liquid PFPE dimethacrylate precursor plus functional monomer (where R is an epoxy) plus a photoinitiator over top of it at 3700 rpm for 1 minute to a thickness of about 20 μm. The wafer is then placed in a UV chamber and exposed to UV light (λ=365) for 10 minutes under a nitrogen purge. The thicker PDMS layer is then placed on top of the 20-μm thick layer and aligned in the desired area to form a seal. The layers are then placed in an oven and allowed to heat at 65° C. for 2 hours. The thin layer is then trimmed and the adhered layers are lifted from the master. Fluid inlet holes and outlet holes are punched using a luer stub. The bonded layers are then placed on a glass slide treated with a silane coupling agent, aminopropyltriethoxy silane, and allowed to heat at 65° C. for 15 hours permanently bonding the device to the glass slide. Small needles can then be placed in the inlets to introduce fluids and to actuate membrane valves as reported by Unger M. et al. Science. 2000, 288, 113-6.

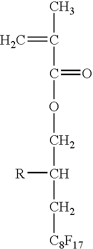

### Example 27

### Photocuring with Functional Monomers Available Post Cure Attachment of a Biomolecule

A liquid PFPE dimethacrylate precursor is blended with a monomer having the structure shown below (where R is an amine group) and blended with 1 wt % of a free radical photoinitiator and poured over a microfluidics master containing 100-μm features in the shape of channels. A PDMS mold is used to contain the liquid in the desired area to a thickness of about 3 mm. The wafer is then placed in a UV chamber and exposed to UV light (λ=365) for 10 minutes under a nitrogen purge. The fully cured layer is then removed from the master and inlet holes are punched using a luer stub. Separately a second master containing 100-μm features in the shape of channels is spin coated with a small drop of the liquid PFPE precursor plus functional (where R is an epoxy group) over top of it at 3700 rpm for 1 minute to a thickness of about 20 μm. The wafer is then placed in a UV chamber and exposed to UV light (λ=365) for 10 minutes under a nitrogen purge. The thicker layer is then placed on top of the 20-μm thick layer and aligned in the desired area to form a seal. The layers are then placed in an oven and allowed to heat at 65° C. for 2 hours. The thin layer is then trimmed and the adhered layers are lifted from the master. Fluid inlet holes and outlet holes are punched using a luer stub. The bonded layers are then placed on a glass slide treated with a silane coupling agent, aminopropyltriethoxy silane, and allowed to heat at 65° C. for 15 hours permanently bonding the device to the glass slide. Small needles can then be placed in the inlets to introduce fluids and to actuate membrane valves as reported by Unger, M. et al. Science. 2000, 288, 113-6. An aqueous solution containing a protein functionalized with a free amine is then flowed through the channel which is lined with unreacted epoxy moieties, in such a way that the channel is then functionalized with the protein.

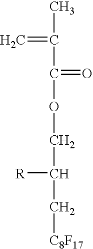

### Example 28

### Photocuring with Latent Functional Groups Available Post Cure Attachment of Charged Species

A liquid PFPE dimethacrylate precursor is blended with a monomer having the structure shown below (where R is an amine group) and blended with 1 wt % of a free radical photoinitiator and poured over a microfluidics master containing 100-μm features in the shape of channels. A PDMS mold is used to contain the liquid in the desired area to a thickness of about 3 mm. The wafer is then placed in a UV chamber and exposed to UV light (λ=365) for 10 minutes under a nitrogen purge. The fully cured layer is then removed from the master and inlet holes are punched using a luer stub. Separately a second master containing 100-μm features in the shape of channels is spin coated with a small drop of the liquid PFPE precursor plus functional (where R is an epoxy group) over top of it at 3700 rpm for 1 minute to a thickness of about 20 μm. The wafer is then placed in a UV chamber and exposed to UV light (λ=365) for 10 minutes under a nitrogen purge. The thicker layer is then placed on top of the 20-μm thick layer and aligned in the desired area to form a seal. The layers are then placed in an oven and allowed to heat at 65° C. for 2 hours. The thin layer is then trimmed and the adhered layers are lifted from the master. Fluid inlet holes and outlet holes are punched using a luer stub. The bonded layers are then placed on a glass slide treated with a silane coupling agent, aminopropyltriethoxy silane, and allowed to heat at 65° C. for 15 hours permanently bonding the device to the glass slide. Small needles can then be placed in the inlets to introduce fluids and to actuate membrane valves as reported by Unger, M. et al. Science. 2000, 288, 113-6. An aqueous solution containing a charged molecule functionalized with a free amine is then flowed through the channel which is lined with unreacted epoxy moieties, in such a way that the channel is then functionalized with the charged molecule.

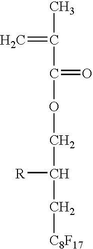

### Example 29

### Fabrication of a PFPE Microfluidic Device Using Sacrificial Channels

A smooth, flat PFPE layer is generated by drawing a doctor's blade across a small drop of the liquid PFPE dimethacrylate precursor across a glass slide. The Slide is then placed in a UV chamber and exposed to UV light (λ=365) for 10 minutes under a nitrogen purge. A scaffold composed of poly(lactic acid) in the shape of channels is laid on top of the flat, smooth layer of PFPE. A liquid PFPE dimethacrylate precursor is with 1 wt % of a free radical photoinitiator and poured over the scaffold. A PDMS mold is used to contain the liquid in the desired area to a thickness of about 3 mm. The apparatus is then placed in a UV chamber and exposed to UV light (λ=365) for 10 minutes under a nitrogen purge. The device can then be heated at 150° C. for 24 hours to degrade the poly(lactic acid) thus revealing voids left in the shape of channels.

### Example 30

### Adhesion of a PFPE Device to Glass Using 185-nm Light

A liquid PFPE dimethacrylate precursor is blended with 1 wt % of a free radical photoinitiator and poured over a microfluidics master containing 100-μm features in the shape of channels. A PDMS mold is used to contain the liquid in the desired area to a thickness of about 3 mm. The wafer is then placed in a UV chamber and exposed to UV light (λ=365) for 10 minutes under a nitrogen purge. Separately a second master containing 100-μm features in the shape of channels is spin coated with a small drop of the liquid PFPE precursor over top of it at 3700 rpm for 1 minute to a thickness of about 20 μm. The wafer is then placed in a UV chamber and exposed to UV light (λ=365) for 10 minutes under a nitrogen purge. The thicker layer is then removed, trimmed, and inlet holes are punched through it using a luer stub. The layer is then placed on top of the 20-μm thick layer and aligned in the desired area to form a seal. The layers are then placed in an oven and allowed to heat at 120° C. for 2 hours. The thin layer is then trimmed and the adhered layers are lifted from the master. Fluid inlet holes and outlet holes are punched using a luer stub. The bonded layers are then placed on a clean, glass slide in such a way that it forms as seal. The apparatus is exposed to 185 nm UV light for 20 minutes, forming a permanent bond between the device and the glass. Small needles can then be placed in the inlets to introduce fluids and to actuate membrane valves as reported by Unger. M. et al. Science. 2000, 288, 113-6.

### Example 31

### “Epoxy Casing” Method to Encapsulate Devices

A liquid PFPE dimethacrylate precursor is blended with 1 wt % of a free radical photoinitiator and poured over a microfluidics master containing 100-μm features in the shape of channels. A PDMS mold is used to contain the liquid in the desired area to a thickness of about 3 mm. The wafer is then placed in a UV chamber and exposed to UV light (λ=365) for 10 minutes under a nitrogen purge. Separately a second master containing 100-μm features in the shape of channels is spin coated with a small drop of the liquid PFPE precursor over top of it at 3700 rpm for 1 minute to a thickness of about 20 μm. The wafer is then placed in a UV chamber and exposed to UV light (λ=365) for 10 minutes under a nitrogen purge. The thicker layer is then removed, trimmed, and inlet holes are punched through it using a luer stub. The layer is then placed on top of the 20-μm thick layer and aligned in the desired area to form a seal. The layers are then placed in an oven and allowed to heat at 120° C. for 2 hours. The thin layer is then trimmed and the adhered layers are lifted from the master. Fluid inlet holes and outlet holes are punched using a luer stub. The bonded layers are then placed on a clean, glass slide in such a way that it forms as seal. Small needles can then be placed in the inlets to introduce fluids and to actuate membrane valves as reported by Unger, M. et al. Science. 2000, 288, 113-6. The entire apparatus can then be encased in a liquid epoxy precursor which is poured over the device allowed to cure. The casing serves to mechanically bind the device the substrate.

### Example 32

### Fabrication of a PFPE Device from a Three-Armed PFPE Precursor

A liquid PFPE precursor having the structure shown below (where the circle represents a linking molecule) is blended with 1 wt % of a free radical photoinitiator and poured over a microfluidics master containing 100-μm features in the shape of channels. A PDMS mold is used to contain the liquid in the desired area to a thickness of about 3 mm. The wafer is then placed in a UV chamber and exposed to UV light (λ=365) for 10 minutes under a nitrogen purge. Separately a second master containing 100-μm features in the shape of channels is spin coated with a small drop of the liquid PFPE precursor over top of it at 3700 rpm for 1 minute to a thickness of about 20 μm. The wafer is then placed in a UV chamber and exposed to UV light (λ=365) for 10 minutes under a nitrogen purge. Thirdly a smooth, flat PFPE layer is generated by drawing a doctor's blade across a small drop of the liquid PFPE precursor across a glass slide. The Slide is then placed in a UV chamber and exposed to UV light (λ=365) for 10 minutes under a nitrogen purge. The thicker layer is then removed, trimmed, and inlet holes are punched through it using a luer stub. The layer is then placed on top of the 20-μm thick layer and aligned in the desired area to form a seal. The layers are then placed in an oven and allowed to heat at 120° C. for 2 hours. The thin layer is then trimmed and the adhered layers are lifted from the master. Fluid inlet holes and outlet holes are punched using a luer stub. The bonded layers are then placed on the fully cured PFPE smooth layer on the glass slide and allowed to heat at 120° C. for 15 hours. Small needles can then be placed in the inlets to introduce fluids and to actuate membrane valves as reported by Unger. M. et al. Science. 2000, 288, 113-6.

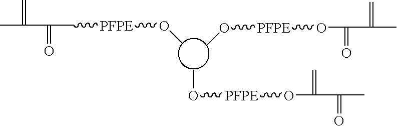

### Example 33

### Photocured PFPE/PDMS Hybrid

A master containing 100-μm features in the shape of channels is spin coated with a small drop of the liquid PFPE dimethacrylate precursor containing photoinitiator over top of it at 3700 rpm for 1 minute to a thickness of about 20 μm. A PDMS dimethacrylate containing photoinitiator is then poured over top of the thin PFPE layer to a thickness of 3 mm. The wafer is then placed in a UV chamber and exposed to UV light (λ=365) for 10 minutes under a nitrogen purge. The layer is then removed, trimmed, and inlet holes are punched through it using a luer stub. The hybrid device is then placed on a glass slide and a seal is formed. Small needles can then be placed in the inlets to introduce fluids.

### Example 34

### Microfluidic Device Formed from Blended Thermally and PhotoCurable Materials

Firstly, a predetermined amount, e.g., 5 grams, of a chain-extended PFPE dimethacrylate containing a small amount of photoinitiator, such as hydroxycyclohexylphenyl ketone, is measured. Next, a 1:1 ratio by weight, e.g., 5 grams, of a chain-extended PFPE diisocyanate is added. Next, an amount, e.g., 0.3 mL, of a PFPE tetrol (Mn˜2000 g/mol) is then added such that there is a stoichiometric amount of —N(C═O)— and OH moieties. The three components are then mixed thoroughly and degassed under vacuum.

Master templates are generated using photolithography and are coated with a thin layer of metal, e.g., Gold/Palladium, using an Argon plasma. Thin layers for devices are spin coated at 1500 rpm from the PFPE blend onto patterned substrates. A thin, flat (non patterned), layer also is spin coated. Separately, thicker layers are cast onto the metal-coated master templates, typically by pooling the material inside, for example, a PDMS gasket. All layers are then placed in a UV chamber, purged with nitrogen for 10 minutes, and photocured for ten minutes into solid rubbery pieces under a thorough nitrogen purge. The layers can then be trimmed and inlet/outlet holes punched. Next the layers are stacked and aligned in registered positions such that they form a conformal seal. The stacked layers are then heated, at 105° C. for 10 minutes. The heating step initiates the thermal cure of the thermally curable material which is physically entangled in the photocured matrix. Because the layers are in conformal contact, strong adhesion is obtained. The two adhered layers can then be peeled from the patterned master or lifted off with a solvent, such as dimethyl formamide, and placed in contact with a third flat, photocured substrate which has not yet been exposed to heat. The three-layer device is then baked for 15 hours at 110° C. to fully adhere all three layers.

According to another embodiment, the thermal cure is activated at a temperature of between about 20 degrees Celsius (C) and about 200 degrees C. According to yet another embodiment, the thermal cure is activated at a temperature of between about 50 degrees Celsius (C) and about 150 degrees C. Further still, the thermal cure selected such that it is activated at a temperature of between about 75 degrees Celsius (C) and about 200 degrees C.

According to yet another embodiment, the amount of photocure substance added to the material is substantially equal to the amount of thermal cure substance. In a further embodiment, the amount of thermal cure substance added to the material is about 10 percent of the amount of photocure substance. According to another embodiment, the amount of thermal cure substance is about 50 percent of the amount of the photocure substance.

### Example 35

### Multicomponent Material for Fabricating Microfluidic Devices

The chemical structure of each component will be described below. In the following example, the first component (Component 1) is a chain extended, photocurable PFPE liquid precursor. The synthesis consists of the chain extension of a commercially available PFPE diol (ZDOL) with a common diisocyanate, isophorone diisocyanate (IPDI), using classic urethane chemistry with an organo-tin catalyst. Following chain extension, the chain is end-capped with a methacrylate-containing diisocyanate monomer (EIM).

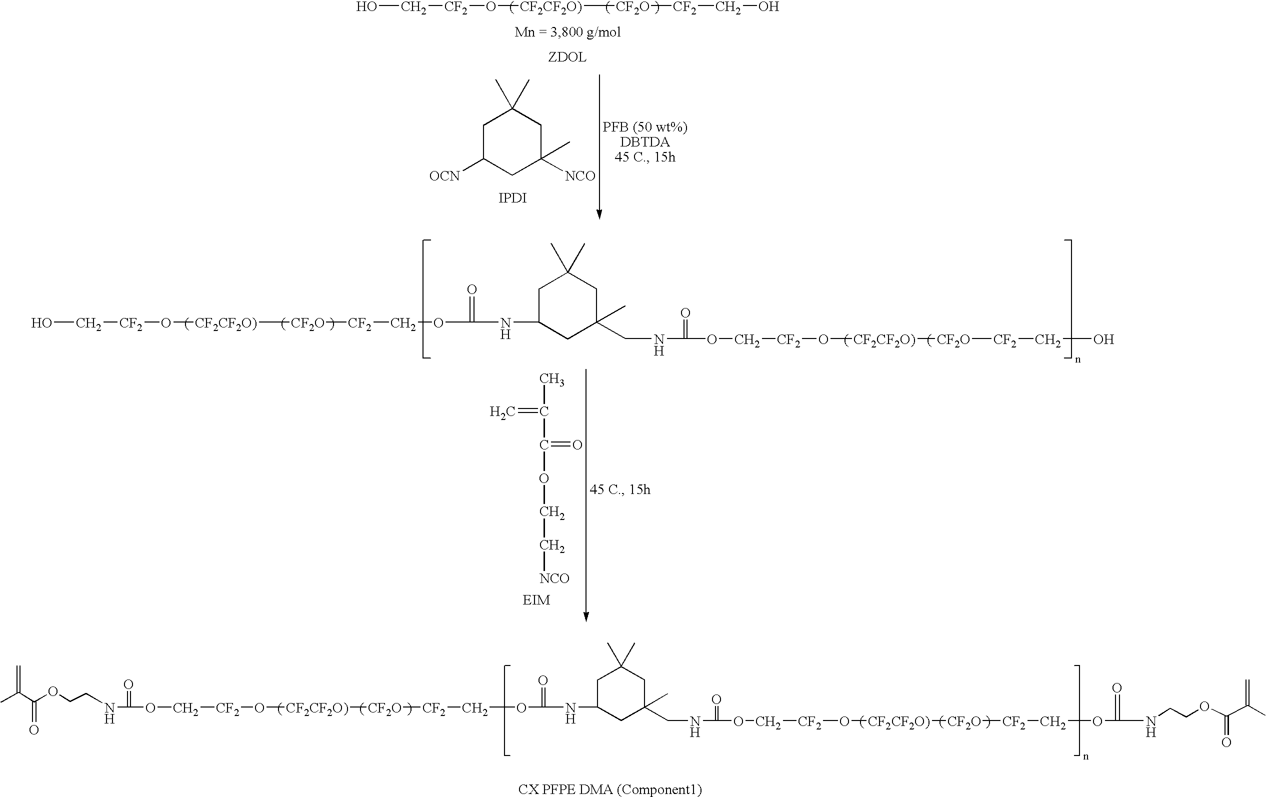

The second component is a chain-extended PFPE diisocyanate. It is made by the reaction of ZDOL with IPDI in a molar ratio such that the resulting polymer chain is capped with isocyanate groups (Component 2a). The reaction again makes use of classic urethane chemistry with an organo-tin catalyst.

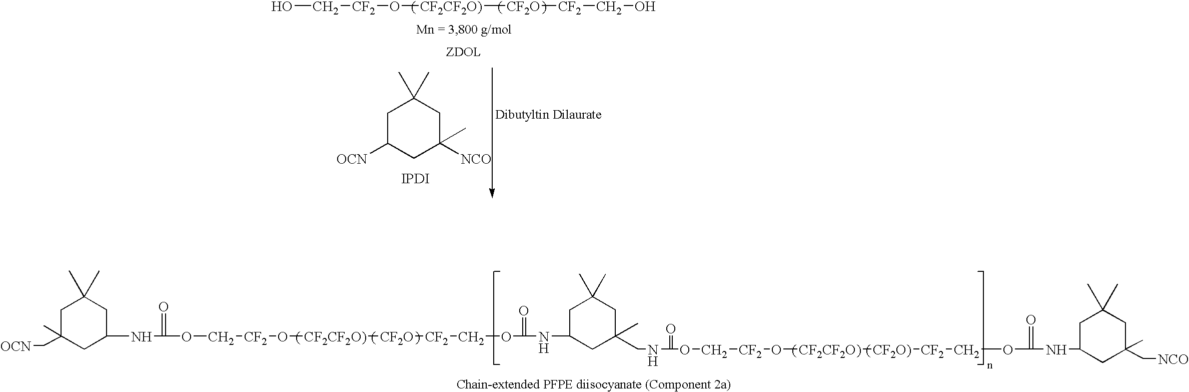

The second part of the thermally curable component is a commercially available perfluoropolyether tetraol with a molecular weight of 2,000 g/mol (Component 2b).

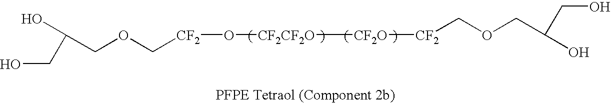

It will be understood that various details of the presently disclosed subject matter can be changed without departing from the scope of the presently disclosed subject matter. Furthermore, the foregoing description is for the purpose of illustration only, and not for the purpose of limitation.

1. A medical implant, comprising:
a medical device configured and dimensioned to be implanted into a patient, wherein the device comprises a reaction product of a first cure and is capable of a second cure.

2. The medical implant of claim 1, wherein the medical device comprises a polymer.

3. The medical implant of claim 2, wherein the polymer comprises a fluorinated polymer.

4. The medical implant of claim 2, wherein the polymer is selected from the group consisting of perfluoropolyether or poly(dimethylsiloxane).

5. The medical implant of claim 1, wherein the first cure comprises exposure to actinic radiation.

6. The medical implant of claim 1, wherein the first cure comprises exposure to thermal energy.

7. The medical implant of claim 1, wherein the second cure comprises exposure to actinic radiation.

8. The medical implant of claim 1, wherein the second cure comprises exposure to thermal energy.

9. The medical implant of claim 1, wherein the medical device comprises a reaction product of a methacrylate.

10. The medical implant of claim 1, wherein the medical device comprises a reaction product of an acrylate.

11. The medical implant of claim 1, wherein the medical device comprises a reaction product of an epoxy.

12. The medical implant of claim 1, wherein the medical device comprises a reaction product of a free radical polymerization.

13. The medical implant of claim 1, wherein the medical device comprises a thermoplastic material.

14. The medical implant of claim 1, wherein the medical device further comprises an organic material.

15. The medical implant of claim 1, wherein the medical device further comprises an imaging agent.

16. The medical implant of claim 1, wherein the medical device further comprises a drug.

17. The medical implant of claim 1, wherein the medical device further comprises a treatment agent.

18. The medical implant of claim 1, wherein the medical device further comprises an antibiotic.

19. The medical implant of claim 1, wherein the medical device further comprises biologic material.

20. The medical implant of claim 1, wherein the medical device comprises a soluble material.

21. The medical implant of claim 1, wherein the medical device comprises a biodegradable material.

22. The medical implant of claim 1, wherein the medical device comprises a hydrophilic material.

23. The medical implant of claim 1, wherein the medical device comprises a hydrophobic material.

24. The medical implant of claim 1, wherein the medical device comprises an inorganic material.

25. The medical implant of claim 1, wherein the medical device comprises a ceramic.

26. The medical implant of claim 1, wherein the medical device comprises a metal.

27. The medical implant of claim 1, wherein the medical device comprises a porogen.

28. The medical implant of claim 1, further comprising a coating on the medical device.

29. The medical implant of claim 28, wherein the coating comprises a fluorinated polymer.

30. The medical implant of claim 29, wherein the fluorinated polymer comprises a perfluoropolyether.

31. A medical implant, comprising:
a base material in combination with a first curable functional group and a second curable functional group.

32. The medical implant of claim 31, wherein the base material comprises a polymer.

33. The medical implant of claim 32, wherein the polymer comprises a fluorinated polymer.

34. The medical implant of claim 32, wherein the polymer is selected from the group consisting of perfluoropolyether or poly(dimethylsiloxane).

35. The medical implant of claim 31, wherein the first curable functional group comprises a functional group that reacts upon exposure to actinic radiation.

36. The medical implant of claim 31, wherein the first curable functional group comprises a functional group that reacts upon exposure to thermal energy.

37. The medical implant of claim 31, wherein the second curable functional group comprises a functional group that reacts upon exposure to actinic radiation.

38. The medical implant of claim 31, wherein the second curable functional group comprises a functional group that reacts upon exposure to thermal energy.

39. The medical implant of claim 31, wherein the first curable functional group comprises a first end-cap, wherein the first end-cap reacts at a first wavelength; and
the second curable functional group comprises a second end-cap, wherein the second end-cap reacts at a second wavelength.

40. The medical implant of claim 31, wherein the first curable functional group comprises a first end-cap, wherein the first end-cap reacts at a first temperature; and
the second curable functional group comprises a second end-cap, wherein the second end-cap reacts at a second temperature.

41. The medical implant of claim 31, wherein the first and second curable functional groups comprises different end-caps.

42. The medical implant of claim 31, wherein the first curable functional group includes a photocurable diurethane methacrylate.

43. The medical implant of claim 31, wherein the first curable functional group includes a diisocyanate.

44. The medical implant of claim 31, wherein the first curable functional group includes a diepoxy.

45. The medical implant of claim 31, wherein the first curable functional group includes a diamine.

46. The medical implant of claim 31, wherein the second functional group includes a photocurable diepoxy.

47. The medical implant of claim 31, wherein the second curable functional group includes a tetrol.

48. The medical implant of claim 31, further comprising a third curable functional group.

49. The medical implant of claim 48, wherein the first curable functional group includes a photocurable diurethane methacrylate, the second curable functional group includes a diisocyanate, and the third curable functional group includes a tetrol.

50. The medical implant of claim 48, wherein the first curable functional group includes a photocurable diurethane methacrylate, the second curable functional group includes a diepoxy, and the third curable functional group includes a diamine.

51. The medical implant of claim 31, wherein the first curable functional group includes a photocurable diurethane methacrylate and the second curable functional group includes a photocurable diepoxy.

52. The medical implant of claim 31, wherein the first curable functional group includes a photocurable diurethane methacrylate and the second curable functional group includes a diisocyanate.

53. An apparatus, comprising:
a medical device; and
a coating on the medical device wherein the coating is a base material in combination with a photocurable functional group and a thermal curable functional group.

54. The apparatus of claim 53, wherein the coating includes a patterned texture on a surface of the coating.

55. The apparatus of claim 54, wherein the patterned texture is configured and dimensioned to interface with a biological tissue.

56. The apparatus of claim 54, wherein the patterned texture reduces wettability of the surface.

57. The apparatus of claim 54, wherein the patterned texture reduces bio-fouling of the surface.

58. The apparatus of claim 54, wherein the patterned texture comprises structures of between about 1 nm and about 500 nm protruding from or recessed into the surface.

59. The apparatus of claim 54, wherein the patterned texture comprises structures of less than about 1 micron protruding from or recessed into the surface.

60. The apparatus of claim 54, wherein the patterned texture comprises structures of between about 5 micron and about 10 micron protruding from or recessed into the surface.

61. The apparatus of claim 54, wherein the patterned texture comprises a repetitive pattern.

62. The apparatus of claim 61, wherein the repetitive pattern is substantially a repeated diamond shaped pattern.

63. The apparatus of claim 53, wherein the coating comprises a fluorinated polymer.

64. The apparatus of claim 63, wherein the coating comprises perfluoropolyether.

65. An apparatus, comprising:
a medical device; and
a patterned texture configured on a surface of the medical device.

66. The apparatus of claim 65, wherein the patterned texture is configured and dimensioned to interface with a biological tissue.

67. The apparatus of claim 65, wherein the patterned texture reduces wettability of the surface.

68. The apparatus of claim 65, wherein the patterned texture reduces bio-fouling of the surface.

69. The apparatus of claim 65, wherein the patterned texture comprises structures of between about 1 nm and about 500 nm protruding from or recessed into the surface.

70. The apparatus of claim 65, wherein the patterned texture comprises structures of less than about 1 micron protruding from or recessed into the surface.

71. The apparatus of claim 65, wherein the patterned texture comprises structures of between about 5 micron and about 10 micron protruding from or recessed into the surface.

72. The apparatus of claim 65, wherein the patterned texture comprises a repetitive pattern.

73. The apparatus of claim 72, wherein the repetitive pattern is substantially a repeated diamond shaped pattern.

74. An artificial joint, comprising:
a base material comprising a photocurable functional group and a thermal curable functional group, wherein the base material is configured to replace or augment a portion of a natural joint.

75. The artificial joint of claim 74, wherein the base material is configured and dimensioned to replace an articular surface of the joint.

76. The artificial joint of claim 74, wherein the base material is configured and dimensioned to replace a structural component of a natural joint.

77. A medical repair device, comprising:
a base material comprising a photocurable functional group and a thermal curable functional group, wherein the base material is configured as a patch to interface with a biologic tissue.

78. The medical repair device of claim 77, wherein the biologic tissue comprises lung tissue.

79. The medical repair device of claim 77, wherein the biologic tissue comprises vascular tissue.

80. The medical repair device of claim 77, wherein the biologic tissue comprises skeletal tissue.

81. The medical repair device of claim 77, wherein the biologic tissue comprises an organ.

82. The medical repair device of claim 77, wherein the biologic tissue comprises bladder tissue.

83. A method of repairing a joint, comprising:
forming a component of a joint from a base material, wherein the base material comprises a first curable functional group and a second curable functional group, wherein the component of the joint is formed by treating the base material with a first cure such that the first curable functional group is activated; and
treating the component of the joint with a second cure, wherein the second cure activates the second curable functional group.

84. The method of claim 83, further comprising:
before said treating the component of the joint with a second cure, implanting the component to an implant site in a patient.

85. The method of claim 84, wherein during said second cure, the component binds with biologic tissue near the implant site.

86. The method of claim 84, wherein during said second cure, the component binds with a polymeric material associated with the implant site.

87. A method of repairing a tissue, comprising:
forming a patch from a base material, wherein the base material comprises a first curable functional group and a second curable functional group, wherein the patch is formed by treating the base material with a first cure such that the first curable functional group is activated;
applying the patch to a tissue having a defect; and
treating the patch with a second cure, wherein the second cure activates the second curable functional group.

88. The method of claim 87, wherein said treating the patch with a second cure binds the patch with tissue to be treated.

89. The method of claim 87, wherein said treating the patch with a second cure binds the patch with a second polymeric material associated with the tissue to be treated.

90. A method of biologic tissue repair, comprising:
forming a repair component from a base material, wherein the base material comprises a first curable functional group and a second curable functional group, wherein the repair component is formed by treating the base material with a first cure such that the first curable functional group is activated; and
treating the repair component with a second cure, wherein the second cure activates the second curable functional group.

91. The method of claim 90, further comprising:
before said treating of the repair component with a second cure, implanting the repair component into a patient.

92. The method of claim 91, wherein during said second cure, the repair component binds with biologic tissue near an implant site.

93. A method of making a medical device, comprising:
forming a first component of a medical device from a base material, wherein the base material comprises a first curable functional group and a second curable functional group, wherein the first component of the medical device is formed by treating a first quantity of the base material with a first cure such that the first curable functional group is activated;
forming a second component of the medical device from a second quantity of the base material by treating the second quantity with a first cure such that the first curable functional group is activated;
positioning the second component with respect to the first component; and
treating the combined first and second components with a second cure, wherein the second cure activates the second curable functional groups of the components and couples the first and second components together.

94. The method of claim 93, wherein the medical device is made in situ.

95. The method of claim 93, wherein the medical device is made in vitro.

96. The method of claim 93, wherein the medical device is selected from the group consisting of an orthopedic device, a vascular device, a surgical device, a wound repair device, an ocular device, an auditory device, a percutaneous device, an external fixation device, a cosmetic augmentation device, an organ scaffold device, a respiratory device, a gastrointestinal device, a digestive device, an excretion device, and a dermatological device.

97. A method of patching a device, comprising:
forming a patch from a base material, wherein the base material comprises a first curable functional group and a second curable functional group, wherein the patch is formed by treating the base material with a first cure such that the first curable functional group is activated;
applying the patch to a device having a defect; and
treating the patch with a second cure, wherein the second cure activates the second curable functional group and couples the patch with the device.
